(19) United States
(12) Patent Application Publication
(10) Pub. No.: US 2025/0325569 A1
(43) Pub. Date: Oct. 23, 2025

(54) COMPOSITIONS AND METHODS OF MAKING AND USE THEREOF

(71) Applicant: Ohio State Innovation Foundation, Columbus, OH (US)

(72) Inventors: Kou-San JU, Dublin, OH (US); Jake WILSON, Columbus, OH (US)

(21) Appl. No.: 18/855,727

(22) PCT Filed: Apr. 13, 2023
(86) PCT No.: PCT/US23/18515

§ 371 Date: Oct. 10, 2024

Related U.S. Application Data
(60) Provisional application No. 63/330,602, filed on Apr. 13, 2022.
(60) Provisional application No. 63/488,008, filed on Mar. 2, 2023.

(51) Int. Cl. A61K 31/662; A01N 57/20; A01N 63/22; A01P 1/00; A61K 35/742; C07F 9/38; C12N 1/20; C12R 1/07

(52) U.S. Cl. CPC A61K 31/662; A01N 57/20; A01N 63/22; A01P 1/00; A61K 35/742; C07F 9/3808; C12N 1/205; C12R 2001/07

(57) ABSTRACT

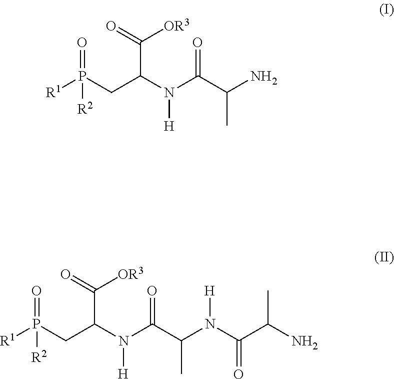

Disclosed herein are compositions and methods of making and use thereof. For example, disclosed herein are compounds comprising a phosphonoalamide of Formula (I) and/or Formula (II). In some examples, the compound is a salt, such as a salt form of Formula (I) and/or Formula (II) with a counterion. Also disclosed herein are compositions comprising any of the compounds disclosed herein, such as pharmaceutical and/or agricultural compositions. Also disclosed herein are methods of use of any of the compounds and/or compositions disclosed herein, for example to control an undesirable population, such as a microbe (e.g., bacteria). Also disclosed herein are methods of reducing the activity of bacteria; reducing bacterial population; killing bacteria; treating, preventing, inhibiting, and/or ameliorating a disease or disorder in a plant or a subject in need thereof, such as a microbial (e.g., bacterial) infection; or a combination thereof using any of the compounds and/or compositions disclosed herein.

### CROSS-REFERENCE TO RELATED APPLICATIONS

This application claims the benefit of priority to U.S. Provisional Application No. 63/330,602 filed Apr. 13, 2022 and U.S. Provisional Application No. 63/488,008 filed Mar. 2, 2023, each of which is hereby incorporated herein by reference in its entirety.

### STATEMENT OF GOVERNMENT SUPPORT

This invention was made with government support under Grant No. R01 GM137135 awarded by the National Institutes of Health. The government has certain rights in the invention.

### BACKGROUND

Some phosphonate natural products, including isolates from naturally-occurring microorganisms, have been shown to have inhibitory activities. The inhibitory activities underly their development as antibiotics and pesticides. Most bio-active phosphonate natural products have been isolated from Actinobacteria. Many plant, animal, and insect pathologies have poor or no modalities of control and new compositions are needed. The compositions and methods discussed herein address these and other needs.

### SUMMARY

In accordance with the purposes of the disclosed compositions and methods as embodied and broadly described herein, the disclosed subject matter relates to compositions and methods of making and use thereof.

For example, disclosed herein are compounds comprising a phosphonoalamide of Formula I and/or Formula II:

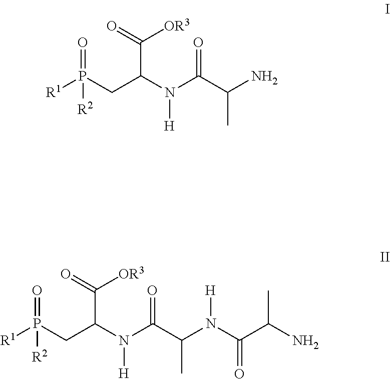

wherein
R¹ is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{a};
R² is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{b};
R³ is hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from H, or substituted or unsubstituted C₁-C₈alkyl, or substituted or unsubstituted C₁-C₅ acyl; or a derivative or salt thereof.

In some examples, the compound is selected from the group consisting of:

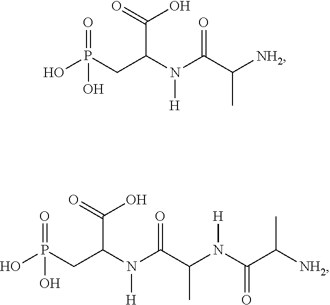 [SMILES: CC(N)C(=O)NC(CP(=O)(O)O)C(=O)O ; CC(N)C(=O)NC(C)C(=O)NC(CP(=O)(O)O)C(=O)O]

derivatives or salts thereof, and combinations thereof.

In some examples, the compound is a salt. In some examples, the compound is a salt form of Formula I and/or Formula II with a counterion.

In some examples, the compound comprises an agriculturally acceptable salt thereof and/or a pharmaceutically acceptable salt thereof.

Also disclosed herein are compositions comprising any of the compounds disclosed herein. In some examples, the composition comprises a pharmaceutical composition, an agricultural composition, or a combination thereof. In some examples, the composition further comprises a solvent, a carrier, an excipient, or a combination thereof. In some examples, the composition further comprises an agriculturally acceptable adjuvant or carrier. In some examples, the composition is formulated for delivery to a plant or animal.

Also disclosed herein are methods of use of any of the compounds and/or compositions disclosed herein. In some examples, the methods comprise controlling an undesirable population. In some examples, the undesirable population comprises a microbe, such as bacteria.

Also disclosed herein are methods of reducing the activity of bacteria; reducing bacterial population; killing bacteria; treating, preventing, inhibiting, and/or ameliorating a disease or disorder in a plant or a subject in need thereof, such as a microbial (e.g., bacterial) infection; or a combination thereof using any of the compounds and/or compositions disclosed herein.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Composition of the contaminated genome by genera. The inner pie chart shows the proportion of contigs attributed to each genus, while the outer pie chart shows the proportion of bases attributed to each genus.

FIG. 2. B. velezensis phosphonoalamide biosynthetic gene cluster. Dashed lines indicate proposed cluster boundaries. Proteins encoded by M. abscessus are at least 97% identical to those encoded by B. velezensis.

FIG. 3. ³¹P NMR spectra from phosphonate production screen (top) and timecourse experiments (bottom).

FIG. 4. Methods used in the isolation of phosphonate natural products, from beginning (top) to end (bottom).

FIG. 5. 2D NMR correlations utilized in structure elucidation of isolated compounds.

FIG. 6. ¹H-¹⁵N HMBC spectrum of phosphonoalamide E show that the methylene protons of phosphonoalanine (δ_{H}≈2 ppm) are correlated to an amide nitrogen (δ≈125 ppm), demonstrating that the compound is a C-terminal phosphonopeptide.

FIG. 7. ¹H-¹⁵N HMBC spectrum of phosphonoalamide F (bottom) show that the methylene protons of phosphonoalanine (δ_{H}≈2 ppm) are correlated to an amide nitrogen (δ_{N}≈125 ppm), demonstrating that the compound is a C-terminal phosphonopeptide.

FIG. 8. Tandem MS data for phosphonoalanine. Structure of isolated compound and observed fragment ions and tandem mass spectrum, annotated to highlight conserved fragmentation patterns, are shown.

FIG. 9. Tandem MS data for phosphonoalamide E. Structure of isolated compound and observed fragment ions and tandem mass spectrum, annotated to highlight conserved fragmentation patterns, are shown.

FIG. 10. Tandem MS data for phosphonoalamide F. Structure of isolated compound and observed fragment ions and tandem mass spectrum, annotated to highlight conserved fragmentation patterns, are shown.

FIG. 11. Extracted ion chromatograms of FDAA-derivatized amino acids. Left column top to bottom: L-alanine, D-alanine, phosphonoalamide E hydrolysate, phosphonoalamide F hydrolysate. Right column top to bottom: DL-phosphonoalanine, L-phosphonoalanine, phosphonoalamide F hydrolysate.

FIG. 12. Diversity of phosphonoalanine cassette containing biosynthetic gene clusters encoded by microbes of the phylum Firmicutes. Sequence similarity network (SSN) of phosphoenolpyruvate mutase (PepM) sequences using an 80% identity threshold. Nodes are colored by genus of the producing organism: green, Bacillus; red, Mycobacteroides; blue, Paenibacillus; light purple, Clostridia; dark purple Oscillospiraceae; orange, Oscillibacter; yellow, Abyssisolibacter. Black star denotes the phosphonoalamide E-F producer Bacillus velezensis NRRL B-41580

FIG. 13. Methods used in the isolation of phosphonate natural products, from beginning (top) to end (bottom).

FIG. 14. Structures of purified compounds. Red arrows indicate ¹H-¹⁵N HMBC correlations. Table 11 summarizes all of the 2D NMR correlations observed from homo- and heteronuclear experiments.

FIG. 15. ¹H-¹⁵N HMBC spectrum of phosphonoalamide E shows that the methylene protons of phosphonoalanine (δ≈2 ppm) are correlated to an amide nitrogen (δ_{N}≈125 ppm), demonstrating that phosphonoalanine is at the carboxy-terminus.

FIG. 16. ¹H-¹⁵N HMBC spectrum of phosphonoalamide F shows that the methylene protons of phosphonoalanine (8H≈2 ppm) are correlated to an amide nitrogen (δ_{N}≈125 ppm), demonstrating that phosphonoalanine is at the carboxy-terminus.

FIG. 17. Diversity of phosphonoalanine-encoding Firmicutes and their biosynthetic gene clusters. 16S rRNA gene phylogenetic tree with Clostridium kluyveri as the outgroup. B. velezensis NRRL B-41580, is labeled red. Colors of the inner ring indicate genera, while those of the outer ring reflect biosynthetic gene cluster type.

FIG. 18. The same colors from FIG. 17 are used to denote gene-cluster families within the phosphoenolpyruvate mutase sequence similarity network (80% identity cutoff). Their corresponding pepM neighborhoods, with biosynthetic gene cluster genes annotated and labeled, are shown. The complete list of strains is provided in Table 12.

FIG. 19. Synteny of the phosphonate/phosphinate biosynthetic gene cluster neighborhoods between Bacillus velezensis NRRL B-41580 and Mycobacterium abscessus subsp. Mussiliense.

FIG. 20. Synteny of the phosphonate/phosphinate biosynthetic gene clusters neighborhoods from Streptomyces sp. NRRL B-2790 and Bacillus velezensis NRRL B-41580. Numerical values denote percent shared identity between close homologs.

FIG. 21. Phosphonate/phosphinate production from Bacillus subtilis NRRL B-4247 grown in different media. Baffled flasks containing NB (nutrient broth), RM medium (RM), R2AS medium (R2AS), GUBC medium (GUBC), or tryptic soy broth (TSB) were inoculated with starter culture. Cultures were incubated on a rotary shaker for 3 days. Culture supernatants were concentrated and analyzed by ³¹P NMR. Signals with chemical shifts>8 ppm are putative phosphonate/phosphinate compounds.

FIG. 22. Phosphonate/phosphinate production from bacillus velezensis NRRL B-41580 grown in different media. Baffled flasks containing NB (nutrient broth), RM medium (RM), R2AS medium (R2AS), GUBC medium (GUBC), or tryptic soy broth (TSB) were inoculated with starter culture. Cultures were incubated on a rotary shaker for 3 days. Culture supernatants were concentrated and analyzed by ³¹P NMR. Signals with chemical shifts>8 ppm are putative phosphonate/phosphinate compounds.

FIG. 23. Phosphonate/phosphinate production from bacillus swezeyi NRRL B-41282 grown in different media. Baffled flasks containing NB (nutrient broth), RM medium (RM), R2AS medium (R2AS), GUBC medium (GUBC), or tryptic soy broth (TSB) were inoculated with starter culture. Cultures were incubated on a rotary shaker for 3 days. Culture supernatants were concentrated and analyzed by ³¹P NMR. Signals with chemical shifts>8 ppm are putative phosphonate/phosphinate compounds.

FIG. 24. Phosphonate/phosphinate production from bacillus swezeyi NRRL B-41294 grown in different media. Baffled flasks containing NB (nutrient broth), RM medium (RM), R2AS medium (R2AS), GUBC medium (GUBC), or tryptic soy broth (TSB) were inoculated with starter culture. Cultures were incubated on a rotary shaker for 3 days. Culture supernatants were concentrated and analyzed by ³¹P NMR. Signals with chemical shifts>8 ppm are putative phosphonate/phosphinate compounds.

FIG. 25. Timecourse of phosphonate/phosphinate production for Bacillus velezensis NRRL B-41580.

FIG. 26. ¹H NMR spectrum of compound 1 in 100% D2O.

FIG. 27. ³¹P NMR spectrum of compound 1 in 100% D2O.

FIG. 28. LC-HRMS data of compound 2.

FIG. 29. ³¹P NMR spectrum of compound 2 in 100% D₂O.

FIG. 30. ¹H NMR spectrum of compound 2 in 100% D₂O.

FIG. 31. ¹³C NMR spectrum of compound 2 in 90% H₂O/10% D₂O.

FIG. 32. ¹³C DEPT 135 spectrum of compound 2 in 90% H₂O/10% D₂O.

FIG. 33. ¹H NMR spectrum of compound 2 in 90% H₂O/10% D₂O.

FIG. 34. ¹H coupled-³¹P NMR spectrum of compound 2 in 90% H₂O/10% D₂O.

FIG. 35. ¹H-³¹P HMBC spectrum of compound 2 in 90% H₂O/10% D₂O.

FIG. 36. ¹H-¹H COSY spectrum of compound 2 in 90% H₂O/10% D₂O.

FIG. 37. ¹H-¹H TOCSY spectrum of compound 2 in 90% H₂O/10% D₂O.

FIG. 38. ¹H-¹³C HSQC spectrum of compound 2 in 90% H₂O/10% D₂O.

FIG. 39. ¹H-¹³C HMBC spectrum of compound 2 in 100% D₂O.

FIG. 40. ¹H-¹⁵N HSQC spectrum of compound 2 in 90% H₂O/10% D₂O.

FIG. 41. ¹H-¹⁵N HMBC spectrum of compound 2 in 90% H₂O/10% D₂O.

FIG. 42. LC-HRMS of compound 3 (positive mode).

FIG. 43. ¹H NMR spectrum of compound 3 in 100% D₂O.

FIG. 44. ¹³C NMR spectrum of compound 3 in 90% H₂O/10% D₂O.

FIG. 45. ¹³C DEPT 135 spectrum of compound 3 in 90% H₂O/10% D₂O.

FIG. 46. ³¹P NMR spectrum of compound 3 in 100% D₂O.

FIG. 47. ¹H NMR spectrum of compound 3 in 90% H₂O/10% D₂O.

FIG. 48. ¹H coupled-³¹P NMR spectrum of compound 3 in 90% H₂O/10% D₂O.

FIG. 49. ¹H-³¹P HMBC spectrum of compound 3 in 100% D₂O.

FIG. 50. ¹H-¹H COSY spectrum of compound 3 in 90% H₂O/10% D₂O.

FIG. 51. ¹H-¹H TOCSY spectrum of compound 3 in 90% H₂O/10% D₂O.

FIG. 52. ¹H-¹³C HSQC spectrum of compound 3 in 90% H₂O/10% D₂O.

FIG. 53. ¹H-¹³C HMBC spectrum of compound 3 in 100% D₂O.

FIG. 54. ¹H-¹⁵N HSQC spectrum of compound 3 in 90% H₂O/10% D₂O.

FIG. 55. ¹H-¹⁵N HMBC spectrum of compound 3 in 90% H₂O/10% D₂O.

FIG. 56. Marfey's analysis for absolution configuration determination of alanine.

FIG. 57. Marfey's analysis for absolution configuration determination of phosphonoalanine.

FIG. 58. Marfey's analysis for absolute configuration determination of phosphonoalanine from phosphonoalamide E.

FIG. 59. MS/MS spectrum of compound 2 (positive mode).

FIG. 60. MS/MS spectrum of compound 3 (positive mode).

FIG. 61. LC-MS of cultures for phosphonates. EIC of phosphonoalanine (m/z 170.0212 [M+H]⁺). EIC of phosphonoalamide E (m/z 241.0577 [M+H]⁺). EIC of phosphonoalamide F (m/z 312.0946 [M+H]⁺).

### DETAILED DESCRIPTION

The compositions, methods, and systems described herein may be understood more readily by reference to the following detailed description of specific aspects of the disclosed subject matter and the Examples included therein.

Before the present compositions, methods, and systems are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%. e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, Me refers to a methyl group; OMe refers to a methoxy group; and i-Pr refers to an isopropyl group.

As used herein, agriculturally acceptable salts and esters refer to salts and esters that exhibit herbicidal activity, or that are or can be converted in plants, water, or soil to the referenced herbicide. Exemplary agriculturally acceptable esters are those that are or can be hydrolyzed, oxidized, metabolized, or otherwise converted, e.g., in plants, water, or soil, to the corresponding carboxylic acid which, depending on the pH, may be in the dissociated or undissociated form.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), birds, and insects. “Subject” can also include a mammal, such as a primate or a human. Thus, the subject can be a human or veterinary patient. The term “patient” refers to a subject under the treatment of a clinician, e.g., physician.

As used herein, antimicrobials include, for example, antibacterials, antifungals, and antivirals. As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, “reduce” or other forms of the word, such as “reducing” or “reduction,” refers to lowering of an event or characteristic (e.g., microbe population/infection). It is understood that the reduction is typically in relation to some standard or expected value, in other words it is relative, but that it is not always necessary for the standard or relative value to be referred to. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of microbial infections, “treating,” “treat,” and “treatment” as used herein, refers to partially or completely inhibiting or reducing the microbial infections which the subject is suffering. In one embodiment, this term refers to an action that occurs while a patient is suffering from, or is diagnosed with, the microbial infections, which reduces the severity of the condition, or retards or slows the progression of the condition. Treatment need not result in a complete cure of the condition; partial inhibition or reduction of the microbial infections is encompassed by this term.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “molecular weight” refers to number average molecular weight as measured by ¹H NMR spectroscopy, unless indicated otherwise.

As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

As used herein, the term “encapsulation,” or grammatical equivalent, refers to the process of confining an individual nucleic acid molecule within a nanoparticle.

As used herein, “expression” of a mRNA refers to translation of an mRNA into a peptide (e.g., an antigen), polypeptide, or protein (e.g., an enzyme) and also can include, as indicated by context, the post-translational modification of the peptide, polypeptide or fully assembled protein (e.g., enzyme). In this application, the terms “expression” and “production,” and grammatical equivalent, are used inter-changeably.

As used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. In some examples, the term “nucleic acid” as used herein means natural and synthetic DNA, RNA, oligonucleotides, oligonucleosides, and derivatives thereof. For ease of discussion, such nucleic acids are at times collectively referred to herein as “constructs,” “plasmids,” or “vectors.”

The term “gene” as used in this specification refers to a segment of deoxyribonucleotides (DNA) possessing the information required for synthesis of a functional biological product such as a protein or ribonucleic acid (RNA).

The term “genetic engineering” is used to indicate various methods involved in gene manipulation including isolation, joining, introducing of gene(s) as well as methods to isolate select organisms containing the manipulated gene(s).

As specified herein, the term “DNA construct” refers to a sequence of deoxyribonucleotides including deoxyribonucleotides obtained from one or more sources.

The term “gene expression” refers to efficient transcription and translation of genetic information contained in concerned genes.

The term “recombinant” cells or population of cells refers to cells or population of cells into which an exogenous nucleic acid sequence is introduced using a delivery vehicle such as a plasmid.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl. 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some examples, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-pentoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The term “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z′ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

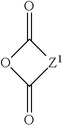

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

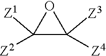

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amino group, an alkyl group, a halide, and the like. Depending upon the groups that are selected, a first group can be incorporated within a second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Disclosed herein are compounds comprising phosphonoalamides or a derivative or salt thereof. In some examples, the compound comprises an agriculturally acceptable salt thereof and/or a pharmaceutically acceptable salt thereof. In some examples, the compound is a peptide. In some examples, the compound is a C-terminal phosphonoalanine-containing peptide. In some examples, the compound is a di-peptide, a tri-peptide, or a combination thereof.

In some examples, the compound comprises a phosphonoalamide of Formula I and/or Formula II:

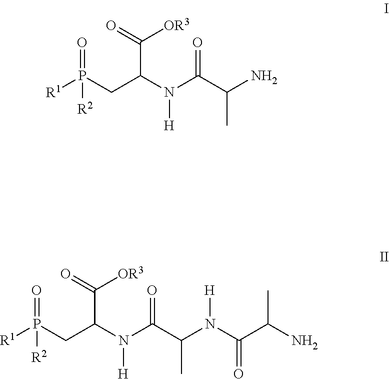

wherein
R′ is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{a};
R² is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{b};
R³ is hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from H, or substituted or unsubstituted C₁-C₈alkyl, or substituted or unsubstituted C₁-C₅ acyl;
or a derivative or salt thereof.

In some examples of Formula I and/or Formula II, R¹ is OR^{a}. In some examples of Formula I and/or Formula II, R² is OR^{b}. In some examples of Formula I or Formula II, R¹ is OR^{a} and R² is OR^{b}.

In some examples of Formula I and/or Formula II, R³ is hydrogen.

In some examples of Formula I and/or Formula II, R¹ is OR^{a} and R³ is hydrogen. In some examples of Formula I and/or Formula II, R² is OR^{b} and R³ is hydrogen. In some examples of Formula I and/or Formula II, R¹ is OR^{a}, R² is ORD, and R³ is hydrogen.

In some examples of Formula I and/or Formula II, R¹ is OH. In some examples of Formula I and/or Formula II, R² is OH. In some examples of Formula I and/or Formula II, R¹ is OH and R² is OH.

In some examples of Formula I and/or Formula II, R¹ is OH and R³ is hydrogen. In some examples of Formula I and/or Formula II, R² is OH and R³ is hydrogen. In some examples of Formula I and/or Formula II, R¹ is OH, R² is OH, and R³ is hydrogen.

In some examples, the compound comprises a phosphonoalamide of Formula I-A and/or Formula II-A:

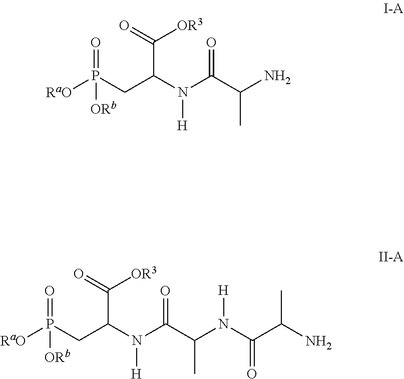

wherein
R^{a}, R^{b}, and R³ are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁ alkylaryl;
or a derivative or salt thereof.

In some examples of Formula I-A and/or Formula II-A, R³ is hydrogen.

In some examples of Formula I-A and/or Formula II-A, R^{a} is hydrogen. In some examples of Formula I-A and/or Formula II-A, R^{b} is hydrogen. In some examples of Formula I-A and/or Formula II-A, R^{a} is hydrogen and R^{b} is hydrogen.

In some examples of Formula I-A and/or Formula II-A, R^{a} and R³ are hydrogen. In some examples of Formula I-A and/or Formula II-A, RD and R³ are hydrogen. In some examples of Formula I-A and/or Formula II-A, R^{a}, R^{b}, and R³ are each hydrogen.

In some examples, the compound is of Formula I-B and/or Formula II-B:

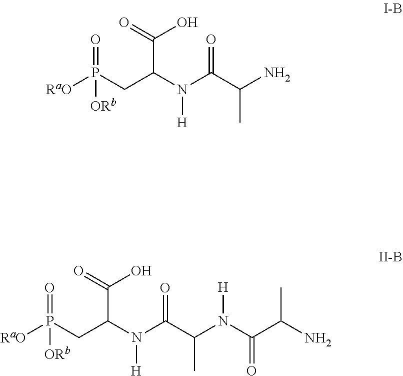

wherein
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
or a derivative or salt thereof.

In some examples of Formula I-B and/or Formula II-B, R^{a} is hydrogen. In some examples of Formula I-B and/or Formula II-B, R^{b} is hydrogen. In some examples of Formula I-B and/or Formula II-B, R^{a} is hydrogen and R^{b} is hydrogen.

In some examples, the compound is of Formula I-C and/or Formula II-C:

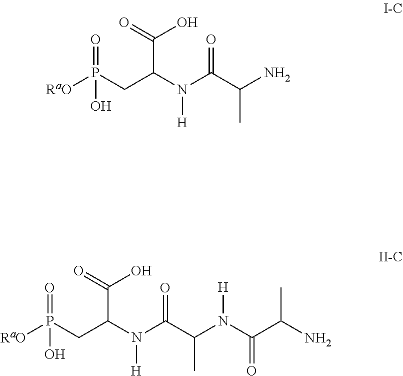

wherein
R^{a} is hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
or a derivative or salt thereof.

In some examples, the compound is of Formula I-D:

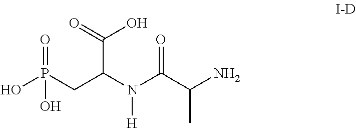

or a derivative or salt thereof.

In some examples, the compound is of Formula I-E:

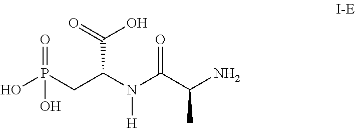

or a derivative or salt thereof.

In some examples, the compound is of Formula II-D:

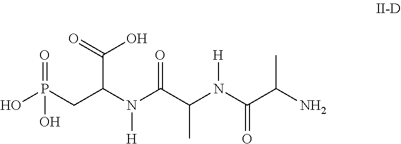 [SMILES: CC(N)C(=O)NC(C)C(=O)NC(CP(=O)(O)O)C(=O)O]

or a derivative or salt thereof.

In some examples, the compound is of Formula II-E:

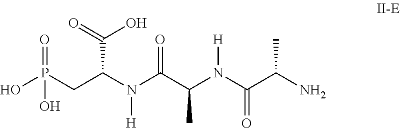 [SMILES: C[C@H](N)C(=O)N[C@@H](C)C(=O)N[C@H](CP(=O)(O)O)C(=O)O]

or a derivative or salt thereof.

In some examples, the compound is selected from the group consisting of:

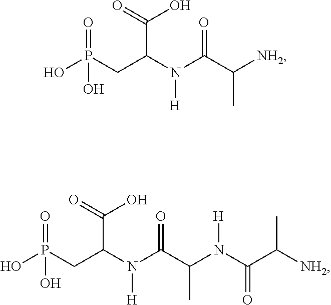 [SMILES: CC(N)C(=O)NC(CP(=O)(O)O)C(=O)O ; CC(N)C(=O)NC(C)C(=O)NC(CP(=O)(O)O)C(=O)O]

derivatives or salts thereof, and combinations thereof.

In some examples, the compound is selected from the group consisting of:

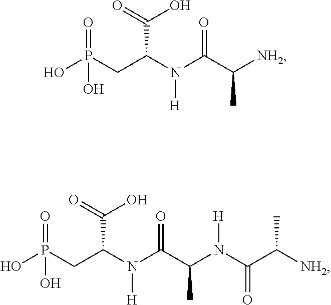 [SMILES: C[C@H](N)C(=O)N[C@H](CP(=O)(O)O)C(=O)O ; C[C@H](N)C(=O)N[C@@H](C)C(=O)N[C@H](CP(=O)(O)O)C(=O)O]

derivatives or salts thereof, and combinations thereof.

In some examples, the compound is a salt. In some examples, the compound is a salt form of Formula I and/or Formula II with a counterion.

In some examples, the compound is a salt form of Formula I and/or Formula II with a counterion, and the salt form of the compound is selected from the group consisting of:

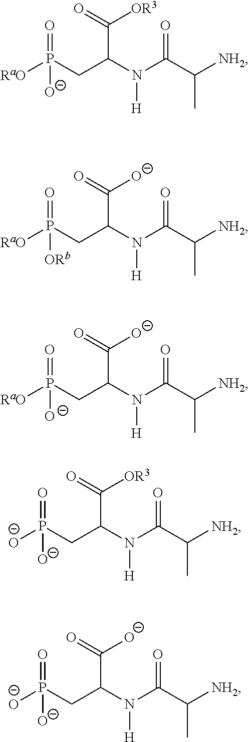

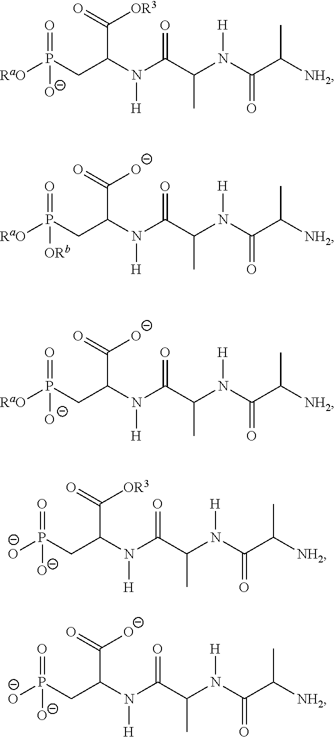

and combinations thereof.

In some examples, the counter ion is as a monovalent, divalent, or trivalent counterion. In some examples, the counterion is selected from the group consisting of sodium, potassium, calcium, lithium, magnesium, manganese, ammonium, iron, and combinations thereof. In some examples, the compound is a potassium salt, a sodium salt, a calcium salt, an iron salt, an ammonium salt, or a combination thereof.

In some examples, the compound comprises an agriculturally acceptable salt thereof and/or a pharmaceutically acceptable salt thereof.

In some examples, the compound is an anti-metabolite.

In some examples, the compound is a Bacillus isolate, or a derivative or salt thereof. In some examples, the compound is an isolate of Bacillus velezensis NRRL B-41580, Bacillus subtilis NRRL B-4247, Bacillus swezeyi NRRL B-41282, Bacillus swezeyi NRRL B-41294, or a combination thereof; or a derivative or salt thereof. In some examples, the compound is a Bacillus velezensis isolate, or a derivative or salt thereof. In some examples, the compound is a B. velezensis NRRL B-41850 isolate, or a derivative or salt thereof.

### Compositions

Also disclosed herein are compositions comprising any of the compounds disclosed herein.

Also disclosed herein are compositions comprising Bacillus and one or more agriculturally acceptable or pharmaceutically acceptable carriers. In some examples, the composition comprises Bacillus velezensis. In some examples, the composition comprises Bacillus velezensis NRRL B-41580, Bacillus subtilis NRRL B-4247, Bacillus swezeyi NRRL B-41282, Bacillus swezeyi NRRL B-41294, or a combination thereof. In some examples, the composition comprises B. velezensis NRRL B-41850.

In some examples, the composition comprises a pharmaceutical composition, an agricultural composition, or a combination thereof.

In some examples, the composition comprises a pesticide. In some examples, the composition comprises an herbicide.

In some examples, the composition exhibits antimicrobial activity. In some examples, the composition results in at least 5 log reduction in a population of microbes.

In some examples, the microbes are one or more microorganisms selected from the group consisting of Erwinia rhapontici, Escherichia coli, Pantoea ananatis, Salmonella enterica, Serratia marcescens, Bacillus subtilis, Bacillus megaterium, Paenibacillus larvae, Pseudomonas aeruginosa, and combinations thereof. In some examples, the microbes are Serratia marcescens and/or Paenibacillus larvae. In some examples, the microbes are one or more microorganisms selected from the group consisting of Escherichia coli K12, Pseudomonas aeruginosa K, Salmonella enterica LT2, Serratia marcescens, Bacillus, and combinations thereof. In some examples, the microbes are one or more microorganisms selected from the group consisting of Erwinia rhapontici, Pantoea ananatis, and combinations thereof.

In some examples, the composition further comprises a solvent, a carrier, an excipient, or a combination thereof. In some examples, the composition further comprises an agriculturally acceptable adjuvant or carrier.

In some examples, the composition is formulated for delivery to a plant or animal.

In some examples, the composition is formulated for delivery to a plant. In some examples, the plant is maize, rice, tomato melon, onion, rhubarb, pea, cucumber, or a combination thereof. In some examples, the composition is formulated for delivery to onions.

In some examples, the composition is formulated for delivery to an animal. In some examples, the animal is a companion animal, livestock, research animal, insect, or human. In some examples, the animal is an insect. In some examples, insect is a bee, such as a honeybee.

Also disclosed herein are nucleic acids encoding any of the compounds or compositions disclosed herein. Also disclosed herein are vectors encoding said nucleic acids. Also disclosed herein are cells comprising said vectors. Also disclosed herein are cells comprising any of the compounds or compositions disclosed herein. In some examples, the cell comprises a Bacillus cell. In some examples, the cell comprises Bacillus velezensis. In some examples, the cell comprises Bacillus velezensis NRRL B-41580, Bacillus subtilis NRRL B-4247, Bacillus swezeyi NRRL B-41282, Bacillus swezeyi NRRL B-41294, or a combination thereof. In some examples, the cell comprises B. velezensis NRRL B-41850.

### Methods of Making and Use

Also disclosed herein are methods of making any of the compounds disclosed herein.

Also disclosed herein are methods of isolating and/or purifying a phosphonate produced by a cell, wherein the phosphonate comprises any of the compounds disclosed herein.

Also disclosed herein are methods of use of any of the compounds, compositions, nucleic acids, vectors, or cells disclosed herein.

For example, also disclosed herein are methods of using any of the compounds, compositions, nucleic acids, vectors, or cells as an antimicrobial, an herbicide, a pesticide, or combination thereof, for example to control (e.g., treat, reduce, inhibit, and/or ameliorate) undesirable population.

In some examples, the methods comprise using any of the compounds, compositions, nucleic acids, vectors, or cells as a pesticide.

In some examples, the methods comprise using any of the compounds, compositions, nucleic acids, vectors, or cells to control (e.g., treat, reduce, inhibit, and/or ameliorate) undesirable population in plants. In some examples, the method comprises contacting the plants or the locus thereof with or applying to the soil or water any of the compounds, compositions, nucleic acids, vectors, or cells. In some examples, the methods further comprise applying an additional pesticide.

In some examples, the undesirable population is a herbicide resistant or tolerant population, a pesticide resistant or tolerant population, an antimicrobial resistant or tolerant population, or a combination thereof. In some examples, the undesirable population comprises bacteria.

Also disclosed herein are methods of reducing the activity of bacteria, the methods comprising exposing the bacteria to an effective amount of any of the compounds, compositions, nucleic acids, vectors, or cells disclosed herein.

Also disclosed herein are methods of reducing bacterial population, the method comprising exposing the bacteria to an effective amount of any of the compounds, compositions, nucleic acids, vectors, or cells disclosed herein.

Also disclosed herein are methods of killing bacteria, the methods comprising exposing the bacteria to an effective amount of any of the compounds, compositions, nucleic acids, vectors, or cells disclosed herein.

Also disclosed herein are methods of treating, preventing, and/or ameliorating a disease or a disorder in a plant or a subject in need thereof, the method comprising administering to the subject a therapeutically effective amount of any of the compounds, compositions, nucleic acids, vectors, or cells disclosed herein.

In some examples, the disease or disorder comprises an infection, such as with an infectious microbe (e.g., bacteria, virus, fungi, protozoa, etc.). In some examples, the disease or disorder comprises a microbial infection.

Also disclosed herein are methods for treating, preventing, inhibiting, and/or ameliorating a microbial infection in a plant or a subject, comprising administering to the plant or subject an effective amount of any of the compounds, compositions, nucleic acids, vectors, or cells disclosed herein.

In some examples, the bacteria comprise Erwinia rhapontici, Escherichia coli, Pantoea ananatis, Salmonella enterica, Serratia marcescens, Bacillus subtilis, Bacillus megaterium, Paenibacillus larvae, Pseudomonas aeruginosa, or a combination thereof. In some examples, the bacteria comprise Serratia marcescens and/or Paenibacillus larvae. In some examples, the bacteria comprise Escherichia coli K12, Pseudomonas aeruginosa K, Salmonella enterica LT2, Serratia marcescens, Bacillus genus, or a combination thereof. In some examples, the bacteria comprise Erwinia rhapontici, Pantoea ananatis, or a combination thereof.

In some examples, the plant is maize, rice, tomato melon, onion, rhubarb, pea, cucumber, or a combination thereof. In some examples, the plant is an onion. In some examples, the bacteria is P. ananatis and plant is an onion.

In some examples, the subject is an animal. In some examples, the animal is a companion animal, livestock, research animal, insect, or human. In some examples, the animal is an insect. In some examples, the insect is a bee, such as a honeybee.

In some examples, the subject is a honeybee and the disease or disorder is American foulbrood (e.g., an infection with Paenibacillus larvae).

In some examples, the compound, composition, nucleic acid, or vector is delivered via cultured Bacillus. In some examples, the compound, composition, nucleic acid, or vector is delivered via cultured Bacillus velezensis. In some examples, the compound, composition, nucleic acid, or vector is delivered via cultured: Bacillus velezensis NRRL B-41580, Bacillus subtilis NRRL B-4247, Bacillus swezeyi NRRL B-41282, Bacillus swezeyi NRRL B-41294, or a combination thereof. In some examples, the compound, composition, nucleic acid, or vector is delivered via cultured B. velezensis NRRL B-41850.

In some examples, the compounds, compositions, nucleic acids, vectors and/or cells can display broad-spectrum antibacterial activity, with strong inhibition against pathogenic microbes, including those responsible for vegetable soft rot (Erwinia rhapontici), onion rot (Pantoea ananatis), and American foulbrood (Paenibacillus larvae).

Erwinia species can cause diseases in woody plants, ornamental flowers, and vegetables. Erwinia rhapontici can cause diseases in rhubarb, celery, peach, onion, kiwifruit, wheat, pea, chickpea, lentil, common bean, lucerne, rye, hyacinth, and tomato.

Pantoea species (family Erwiniaceae) can diseases in plants, insects, animals, and humans.

In plants, Pantoea species are pathogenic to pea, sweet corn, sweet potato, sugarcane, bamboo, wheat, cotton, gypsophila paniculate, rice, beach pea, Chinese taro, beet, onion, switchgrass, netted melon, Sudangrass, eucalyptus, agave, grape, and deepwater rice.

Insects serve as vectors of Pantoea species, including flea beetles (e.g., corn), maggot flies (e.g., blueberry), thrips (e.g., tobacco, cotton), fleahoppers, aphids, pea aphids, wood-boring beetles, locusts, fruit flies, honey bees, Lygus hesperus, Phylloxeras (e.g., grape, pecan), caterpillars, grass grubs, Phlebotomus paputusis, Asian long-horned beetles, Plugioderu versicoloras, mosquitos, pine engravers, leaf cutters, ants, stink bugs, sandflies, and flies.

In non-human animals, Pantoea species colonize horses, brown trout, chickens, rainbow trout, mangrove crab, dolphin fish, chinook salmon, slugs, giant pandas, geese, ostriches, deer, dunnocks, and cattle.

In humans, Pantoea species colonize wounds, fractures, epidermis, respiratory tract, urinary tract, digestive tract, ear, mouth and throat, blood, and lacerations. Colonization of Pantoea species in humans are associated with septic arthritis, osteomyelitis, bacteremia, septicemia, nosocomial infection, peritonitis, sepsis, septic monoarthritis, liver abscess, periodontal disease, pneumonia, respiratory distress, acute hip prosthesis joint infection, corneal infiltration, bacteremia, nosocomial infection, and dacryocystitis.

In plants, Pantoea ananatis is an epiphyte on rice and pineapple. Pantoea ananatis is a pathogen on bamboo, switchgrass, rice, netted melon, onion, Sudangrass, eucalyptus, corn, and agave. Insects serve as vectors of Pantoea ananatis, including thrips (e.g. tobacco, cotton), fleahopper, and Lygus hesperus.

In humans, Pantoea ananatis colonization has been associated with corneal infiltration and bacteremia.

Paenibacillus species can cause diseases in plants, insects, animals, and humans. Paenibacillus species colonize bees and freshwater snails. Freshwater snails are an intermediate host for schistosomiasis. Paenibacillus larvae can cause diseases in bees (e.g., foulbrood in honeybees).

The methods of treatment of the disease or disorder described herein can further include treatment with one or more additional agents. The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

It is understood, however, that the specific dose level for any particular subject will depend upon a variety of factors. Such factors include the age, body weight, general health, sex, and diet of the subject. Other factors include the time and route of administration, rate of excretion, drug combination, and the type and severity of the particular disease or disorder.

The methods, compounds, and compositions as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

### Pharmaceutical Compositions

Also disclosed herein are pharmaceutical compositions comprising any of the compounds or compositions disclosed herein.

In some examples, the pharmaceutical composition is administered to a subject. In some examples, the subject is an animal. In some examples, the animal is a companion animal, livestock, research animal, insect, or human. In some examples, the animal is an insect. In some examples, insect is a bee, such as a honeybee.

In some examples, the disclosed compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

### Pharmaceutical Compositions, Formulations, Methods of Administration, and Kits

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other substances and/or therapies and/or with surgical treatment. These other substances or treatments can be given at the same as or at different times from the compounds or compositions disclosed herein.

In certain examples, compounds and compositions disclosed herein can be locally administered at one or more anatomical sites, such as sites of microbial infection, optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

In some examples, the kit further comprises at least one agent, wherein the compound and the agent are co-formulated.

In some examples, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

### Agricultural Compositions, Formulations, and Methods of Administration

Also disclosed herein are agricultural compositions comprising any of the compounds or compositions disclosed herein, and methods of use thereof.

For example, the compound or composition can be applied to vegetation or an area adjacent the vegetation or applied to soil or water to prevent the emergence or growth of vegetation in an amount sufficient to induce an effect, such as an antimicrobial effect. In some embodiments, compounds or compositions are used in an amount sufficient to induce an antimicrobial effect while still showing good crop compatibility.

The present disclosure also relates to formulations of the compositions and methods disclosed herein. In some embodiments, the formulation can be in the form of a single package formulation including any of the compounds disclosed herein. In some embodiments, the formulation can be in the form of a single package formulation including any of the compounds disclosed herein and further including at least one additive. In some embodiments, the formulation can be in the form of a two-package formulation, wherein one package contains any of the compounds disclosed herein and while the other package contains at least one additive. In some embodiments of the two-package formulation, the formulation including any of the compounds disclosed herein and the formulation including at least one additive are mixed before application and then applied simultaneously. In some embodiments, the mixing is performed as a tank mix (i.e., the formulations are mixed immediately before or upon dilution with water). In some embodiments, the formulation including (a) and the formulation including (b) are not mixed but are applied sequentially (in succession), for example, immediately or within 1 hour, within 2 hours, within 4 hours, within 8 hours, within 16 hours, within 24 hours, within 2 days, or within 3 days, of each other.

In some embodiments, the formulation of any of the compounds disclosed herein is present in suspended, emulsified, or dissolved form. Exemplary formulations include, but are not limited to, aqueous solutions, powders, suspensions, also highly-concentrated aqueous, oily or other suspensions or dispersions, aqueous emulsions, aqueous microemulsions, aqueous suspo-emulsions, oil dispersions, self-emulsifying formulations, pastes, dusts, and materials for spreading or granules.

In some embodiments, the compound or composition is an aqueous solution that can be diluted before use. In some embodiments, the compound or composition is provided as a high-strength formulation such as a concentrate. In some embodiments, the concentrate is stable and retains potency during storage and shipping. In some embodiments, the concentrate is a clear, homogeneous liquid that is stable at temperatures of 54° C. or greater. In some embodiments, the concentrate does not exhibit any precipitation of solids at temperatures of −10° C. or higher. In some embodiments, the concentrate does not exhibit separation, precipitation, or crystallization of any components at low temperatures. For example, the concentrate remains a clear solution at temperatures below 0° C. (e.g., below −5° C., below −10° C., below −15° C.). In some embodiments, the concentrate exhibits a viscosity of less than 50 centipoise (50 megapascals), even at temperatures as low as 5° C.

The compositions and methods disclosed herein can also be mixed with or applied with an additive. In some embodiments, the additive can be diluted in water or can be concentrated. In some embodiments, the additive is added sequentially. In some embodiments, the additive is added simultaneously. In some embodiments, the additive is premixed with the compound.

In some embodiments, the additive is an additional pesticide. For example, the compositions described herein can be applied in conjunction with one or more additional pesticides. The composition can be formulated with the one or more additional pesticides, tank mixed with the one or more additional pesticides, or applied sequentially with the one or more additional pesticides.

In some embodiments, the additional pesticide or an agriculturally acceptable salt or ester thereof is provided in a premixed formulation with the compound.

In some embodiments, the additive includes an agriculturally acceptable adjuvant. Exemplary agriculturally acceptable adjuvants include, but are not limited to, antifreeze agents, antifoam agents, compatibilizing agents, sequestering agents, neutralizing agents and buffers, corrosion inhibitors, colorants, odorants, penetration aids, wetting agents, spreading agents, dispersing agents, thickening agents, freeze point depressants, antimicrobial agents, crop oil, herbicide safeners, adhesives (for instance, for use in seed formulations), surfactants, protective colloids, emulsifiers, tackifiers, and mixtures thereof.

Exemplary agriculturally acceptable adjuvants include, but are not limited to, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphate alcohol ethoxylate; natural primary alcohol (C₁₂-C₁₆) ethoxylate or less, di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8 EO); tallow amine ethoxylate (15 EO); and PEG (400) dioleate-99.

In some embodiments, the additive is a safener, which is an organic compound leading to better crop plant compatibility when applied with a pesticide. In some embodiments, the safener itself is herbicidally active. In some embodiments, the safener acts as an antidote or antagonist in the crop plants and can reduce or prevent damage to the crop plants.

Exemplary surfactants (e.g., wetting agents, tackifiers, dispersants, emulsifiers) include, but are not limited to, the alkali metal salts, alkaline earth metal salts and ammonium salts of aromatic sulfonic acids, for example lignosulfonic acids, phenolsulfonic acids, naphthalenesulfonic acids, and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalene sulfonic acids with phenol and formaldehyde, polyoxyethylene octylphenol ether, ethoxylated isooctyl-, octyl- or nonylphenol, alkylphenyl or tributylphenyl polyglycol ether, alkyl aryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denatured proteins, polysaccharides (e.g., methylcellulose), hydrophobically modified starches, polyvinyl alcohol, polycarboxylates, polyalkoxylates, polyvinyl amine, polyethyleneimine, polyvinylpyrrolidone and copolymers thereof.

Exemplary thickeners include, but are not limited to, polysaccharides, such as xanthan gum, and organic and inorganic sheet minerals, and mixtures thereof.

Exemplary antifoam agents include, but are not limited to, silicone emulsions, long-chain alcohols, fatty acids, salts of fatty acids, organofluorine compounds, and mixtures thereof.

Exemplary antimicrobial agents include, but are not limited to, bactericides based on dichlorophen and benzyl alcohol hemiformal, and isothiazolinone derivatives, such as alkylisothiazolinones and benzisothiazolinones, and mixtures thereof.

Exemplary antifreeze agents include, but are not limited to ethylene glycol, propylene glycol, urea, glycerol, and mixtures thereof.

Exemplary colorants include, but are not limited to, the dyes known under the names Rhodamine B, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108, and mixtures thereof.

Exemplary adhesives include, but are not limited to, polyvinylpyrrolidone, polyvinyl acetate, polyvinyl alcohol, tylose, and mixtures thereof.

In some embodiments, the additive includes a carrier. In some embodiments, the additive includes a liquid or solid carrier. In some embodiments, the additive includes an organic or inorganic carrier. Exemplary liquid carriers include, but are not limited to, petroleum fractions or hydrocarbons such as mineral oil, aromatic solvents, paraffinic oils, and the like or less, vegetable oils such as soybean oil, rapeseed oil, olive oil, castor oil, sunflower seed oil, coconut oil, corn oil, cottonseed oil, linseed oil, palm oil, peanut oil, safflower oil, sesame oil, tung oil and the like or less, esters of the above vegetable oils or less, esters of monoalcohols or dihydric, trihydric, or other lower polyalcohols (4-6 hydroxy containing), such as 2-ethyl hexyl stearate, n-butyl oleate, isopropyl myristate, propylene glycol dioleate, di-octyl succinate, di-butyl adipate, di-octyl phthalate and the like or less, esters of mono, di and polycarboxylic acids and the like, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methyl alcohol, ethyl alcohol, isopropyl alcohol, amyl alcohol, ethylene glycol, propylene glycol, glycerine, N-methyl-2-pyrrolidinone, N,N-dimethyl alkylamides, dimethyl sulfoxide, liquid fertilizers and the like, and water as well as mixtures thereof. Exemplary solid carriers include, but are not limited to, silicas, silica gels, silicates, talc, kaolin, limestone, lime, chalk, bole, loess, clay, dolomite, diatomaceous earth, calcium sulfate, magnesium sulfate, magnesium oxide, ground synthetic materials, pyrophyllite clay, attapulgus clay, kieselguhr, calcium carbonate, bentonite clay, Fuller's earth, cottonseed hulls, wheat flour, soybean flour, pumice, wood flour, walnut shell flour, lignin, ammonium sulfate, ammonium phosphate, ammonium nitrate, ureas, cereal meal, tree bark meal, wood meal and nutshell meal, cellulose powders, and mixtures thereof.

In some embodiments, emulsions, pastes or oil dispersions can be prepared by homogenizing the compound in water by means of wetting agent, tackifier, dispersant or emulsifier. In some embodiments, concentrates suitable for dilution with water are prepared, comprising the compound, a wetting agent, a tackifier, and a dispersant or emulsifier.

In some embodiments, powders or materials for spreading and dusts can be prepared by mixing or concomitant grinding of the compound and optionally a safener with a solid carrier.

In some embodiments, granules (e.g., coated granules, impregnated granules and homogeneous granules) can be prepared by binding the compound to solid carriers.

The compositions disclosed herein can be applied in any known technique for applying pesticides. Exemplary application techniques include, but are not limited to, spraying, atomizing, dusting, spreading, or direct application into water (in-water). The method of application can vary depending on the intended purpose. In some embodiments, the method of application can be chosen to ensure the finest possible distribution of the compositions disclosed herein.

If desired, the compositions can be applied as an in-water application.

When the compositions are used in crops, the compositions can be applied after seeding and before or after the emergence of the crop plants. In some embodiments, when the compositions are used in crops, the compositions can be applied before seeding of the crop plants.

In some embodiments, the compositions disclosed herein are applied to vegetation or an area adjacent the vegetation or applied to soil or water by spraying (e.g., foliar spraying). In some embodiments, the spraying techniques use, for example, water as carrier and spray liquor rates of from 10 liters per hectare (L/ha) to 2000 L/ha (e.g., from 50 L/ha to 1000 L/ha, or from 100 to 500 L/ha). In some embodiments, the compositions disclosed herein are applied by the low-volume or the ultra-low-volume method, wherein the application is in the form of micro granules. In some embodiments, wherein the compositions disclosed herein are less well tolerated by certain crop plants, the compositions can be applied with the aid of the spray apparatus in such a way that they come into little contact, if any, with the leaves of the sensitive crop plants while reaching the undesirable population or the bare soil (e.g., post-directed or lay-by). In some embodiments, the compositions disclosed herein can be applied as dry formulations (e.g., granules, WDGs, etc.) into water.

The compositions and methods disclosed herein can also be used in plants that are resistant to, for instance, pesticides, pathogens, and/or insects. In some embodiments, the compositions and methods disclosed herein can be used in plants that are resistant to one or more pesticides because of genetic engineering or breeding.

In some embodiments, the compositions described herein and other complementary pesticides are applied at the same time, either as a combination formulation or as a tank mix, or as sequential applications.

The compositions and methods may be used in controlling undesirable populations in crops possessing agronomic stress tolerance (including but not limited to drought, cold, heat, salt, water, nutrient, fertility, pH), pest tolerance (including but not limited to insects, fungi and pathogens) and crop improvement traits (including but not limited to yield; protein, carbohydrate, or oil content; protein, carbohydrate, or oil composition; plant stature and plant architecture).

The herbicidal compositions described herein can be used to control herbicide resistant or tolerant populations. The methods employing the compositions described herein may also be employed to control herbicide resistant or tolerant populations. Exemplary resistant or tolerant populations include, but are not limited to, biotypes with resistance or tolerance to multiple herbicides, biotypes with resistance or tolerance to multiple chemical classes, biotypes with resistance or tolerance to multiple herbicide modes-of-action, and biotypes with multiple resistance or tolerance mechanisms (e.g., target site resistance or metabolic resistance).

The present compositions may be formulated and delivered to host plants by methods known in the art, including soil drench via soil drench formulations, seed inoculation via seed inoculation formulations, and plant inoculation via plant inoculation formulations. Seed inoculation formulations can include a carrier such as peat slurry or a film coat consisting of alginate polymers, to protect the compositions from environmental stresses such as desiccation and temperature perturbations. Soil drench or in-furrow composition delivery to plants may be performed by applying the compositions and/or composition formulations in soil before or after planting. Soil drench has several advantages over seed inoculation: 1) prevents the compositions or composition formulations from being inhibited by the chemicals coated on seeds (e.g., fungicides and pesticides) and 2) delivers compositions or composition formulations at higher density without being constrained by seed size. A higher composition or composition formulation concentration is usually required for soil inoculation. Foliar spray and root dipping are also suitable for composition or composition formulation delivery of plants. Plants may be treated at the seedling stage to increase persistence in the plant. In addition, seedling priming, direct seed coating, alginate seed coating, and 12-h coating are within the scope of the present disclosure.

The compositions in the present invention may be formulated and administered to insect hives as a liquid suspension, powder, or solid substrates, such as lipid-based patties.

Liquid formulations may optionally comprise water, sugar syrup and/or other carbohydrate, vitamins, stabilizers, and any other nutrients supportive of bee health. Dry formulations may optionally comprise powdered sugar or other carbohydrate, vitamins, stabilizers, and any other nutrients supportive of bee health. Patty formulations may comprise sugar and/or other carbohydrate, vegetable and/or animal fat, vitamins, stabilizers, and any other nutrients supportive of bee health.

The compositions may be administered as a treatment and/or prophylactically. The compositions may also be administered as a protocol that includes vaccination, phage therapy, the use of lactic acid-producing bacteria.

The formulations optionally include additional foulbrood treatments, such as tylosin tartrate (produced by Elanco, e.g., tylosin A, B, C, and D), and/or Terramycin® (produced by Pfizer, e.g. TM25®, TM50®, TM100®), including Terra-Pro®, and/or the active ingredient of Terramycin®, oxytetracycline HCL. For example, the compounds and compositions disclosed herein can be formulated and/or used in conjunction with the known foulbrood treatments. Therefore, the methods include treatment with one or more of the present compositions and can optionally include additional treatments from previously-known modalities.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Genome Mining Reveals Bacillus as a New Source of the Antimicrobial Phosphonoalamide Natural Products

ABSTRACT. Phosphonic acids are a class of microbial natural products that have potent inhibitory activities. A carbon-phosphorous bond allows phosphonates to mimic primary metabolites containing phosphate esters and carboxylic acids, leading to the inhibition of essential metabolic pathways. Consequently, numerous members of this class have been commercialized as antimicrobials and herbicides. Of microbes with the potential to produce phosphonates, actinobacteria (Class Actinomycetia) have been the focus of recent discovery efforts, due to the genetic diversity of their phosphonate biosynthetic gene clusters (BGCs), and their storied ability to produce pharmaceutically relevant compounds. Despite the presence of phosphonate biosynthetic gene clusters in 5% of genomes in the IMG genomes database, which encompass 9 of 33 bacterial phyla represented, phosphonate metabolism outside of the phylum Actinomycetota are underexplored. Herein, the isolation, chemical characterization, and bioactivity of phosphonopeptides produced by Bacillus velezensis are described.

INTRODUCTION. Phosphonic acids, identifiable by a direct carbon to phosphorous bond, have numerous commercial applications. While utilized in a variety of contexts, many members of this chemical class are known to have potent inhibitory activities. This trait has enabled their use in medicine as antimicrobials, and in agriculture as herbicides. The inhibitory properties of these compounds, which are both synthetic and natural in origin, are generally attributed to their hallmark phosphonate functionality. Chemically analogous to the phosphate esters and carboxylates present in all biological systems, the phosphonate group allows phosphonic acids to function as antimetabolites [1]. By chemically mimicking the native substrate of an essential protein, phosphonate antagonists perturb primary metabolism, ultimately inhibiting growth of the target organism. The increased occurrence of antimicrobial resistant infections, synonymous to the emergence of herbicide resistant weeds, has heightened interest in phosphonic acids as potential solutions to these multifaceted problems [2].

Microbes have proven to be an invaluable source of bioactive and chemically diverse phosphonate natural products [3]. Those that have had commercial success include fosfomycin, an antibiotic used in the treatment of urinary tract infections, and glufosinate, a broad-spectrum herbicide. Logically, the utility of phosphonates has caused considerable interest in their biosyntheses, and research efforts to date have shed some light on the unusual biochemical transformations leading to their production. Perhaps most importantly, biosynthesis of nearly all known phosphonates is initiated by phosphoenolpyruvate mutase (PepM), which catalyzes the isomerization of phosphoenolpyruvate to phosphonopyruvate, forming the characteristic carbon to phosphorous bond [1]. This universally conserved first step allows pepM to serve as a genetic marker of phosphonate biosynthetic potential. Leveraging this knowledge, recent analyses of public sequencing databases have revealed a trove of phosphonate biosynthetic gene clusters (BGCs) encoded by a large number of evolutionarily diverse microorganisms [4]. These biosynthetic gene clusters largely outnumber phosphonates reported in the literature, indicating that many new compounds await discovery.

Of microbes with the potential to produce phosphonates, actinobacteria (Class Actinomycetia) have been the focus of recent discovery efforts, due to the genetic diversity of their biosynthetic gene clusters, and their storied ability to produce pharmaceutically and agriculturally useful compounds |5|. Despite the occurrence of pepM in 5.7% of genomes in the IMG genomes database, which encompass 9 of 33 bacterial phyla represented, phosphonate metabolism outside of the phylum Actinomycetota is significantly less explored [4]. Regardless, the potential utility and physiological importance of phosphonates produced by other microbes should not be overlooked. For instance, rhizocticin is an antifungal phosphonopeptide produced by Bacillus subtilis ATCC 6633 [6]. The active moiety, 1-amino-5-phosphonopentenoic acid (APPA), is an irreversible inhibitor of threonine synthase [7]. Notably, the pathway for de novo threonine biosynthesis is absent in humans, making threonine synthase a promising target for antimicrobial development. Pantaphos, a phosphonate produced by the plant pathogen Pantoea ananatis, was shown to be necessary and sufficient for the hallmark lesions of onion center rot [8]. Ecological importance aside, the general phytotoxicity of pantaphos may enable development as an herbicide. Such discoveries demonstrate the importance of characterizing phosphonates from taxonomically distinct microbes. Further exploring relationships between biological diversity, chemical diversity, and bioactivity may accelerate the discovery of useful phosphonates. Herein, the isolation, chemical characterization, and bioactivity of phosphonopeptides produced by Bacillus velezensis are described.

### Results and Discussion

Genomic Analysis. Recent work from has detailed the discovery of phosphonoalamides A-D, a class of antibacterial phosphonopeptides produced by Streptomyces sp. NRRL B-2790 [9]. The genome mining strategy for this project utilized prior knowledge of phosphonate metabolism, in that the isomerization catalyzed by PepM is energetically unfavorable, and requires a coupling enzyme to drive the reaction towards net phosphonate production [10]. Examining biosynthetic gene clusters lacking a known coupling enzyme revealed an aspartate aminotransferase (aspC) capable of forming phosphonoalanine (PnAla, 2-amino-3-phosphonopropionic acid) from phosphonopyruvate.

Interestingly, a survey of actinobacteria capable of producing PnAla uncovered a phosphonoalamide-like biosynthetic gene cluster encoded by a clinical isolate of the human pathogen Mycobacteroides abscessus subsp. massilience, while all other hits belonged to the genus Streptomyces. This unusual finding prompted further investigation of this cluster. A BLASTp query of the NCBI non-redundant protein database using the PepM sequence from M. abscessus subsp. massilience showed the top hit to be a PepM encoded by multiple strains of Bacillus velezensis. This result seemed improbable, considering these organisms belong to different phyla, which raised concerns about the reported source of the sequencing data in question. The genome of M. abscessus subsp. massilience was subjected to further bioinformatic analyses to verify suspected contamination.

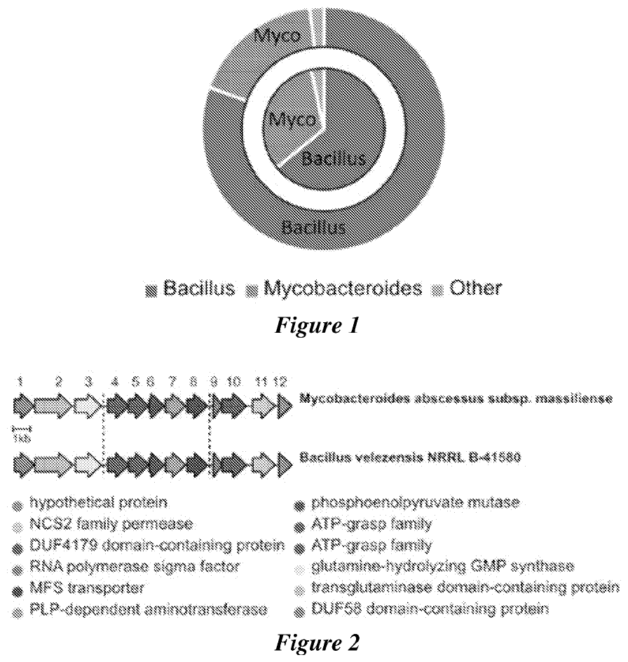

FIG. 1-FIG. 2 illustrate that the contaminated genome of Mycobacteroides abscessus subsp. massilience contains a phosphonoalamide biosynthetic gene cluster from Bacillus velezensis.

The first 2 kb of each contig were used to query the NCBI non-redundant protein database via BLASTn, and the genus of the top hit was recorded. Of the 3,914 contigs making up the assembly, 2,510 were assigned as Bacillus (64.1%), 1,268 as Mycobacteroides (32.4%), 54 as Staphylococcus (1.4%), 2 as Rhodococcus (0.05%), 1 as Rhodoferax (0.02%), and 1 as Streptomyces (0.02%), while 47 (1.2%) returned no hits (FIG. 1). The sizes of these contigs were also considered. Of the 10.7 Mb assembly, 8.6 Mb were attributed to Bacillus (80.6%), 1.9 Mb to Mycobacteroides (17.5%), 97 kb to Staphylococcus (0.9%), 41 kb to Mycobacterium (0.4%), 4 kb to Rhodoferax (0.04%), 3 kb to Rhodococcus (0.03%), and 2 kb to Streptomyces (0.02%) (FIG. 1). Furthermore, 3 16S rRNA genes were present within the genome. Of these, 1 matched to Bacillus velezensis Pilsner 2-2 with 99.93% identity, 1 matched to Staphylococcus epidermidis NRC 113846 with 100% identity, and 1 matched to Mycobacteroides abscessus T84b with 100% identity. These findings show that a genome annotated as M. abscessus in the NCBI database is predominantly comprised of DNA from B. velezensis, corroborating earlier PepM BLASTp results and revealing the source of the phosphonoalamide-like biosynthetic gene cluster shown here (FIG. 2).

The phosphonoalamide-like biosynthetic gene cluster encoded by B. velezensis is highly similar to that of Streptomyces spp. in terms of gene content, but differs slightly in gene architecture. Upstream of pepM are two ATP-grasp family proteins, while directly downstream is an aspartate aminotransferase, followed by an MFS transporter (Table 1). Biosynthesis of phosphonoalanine in Streptomyces sp. NRRL B-2790 was experimentally shown to proceed through transamination of phosphonopyruvate by aspartate aminotransferase, while amino acid ligation is proposed to occur through the activity of ATP-grasp ligases, forming phosphonoalamides A-D [9]. Colocalization of these four genes led to the hypothesis that B. velezensis is capable of producing phosphonoalamide derivatives. Therefore, it was aimed to chemically characterize and evaluate the bioactivity of phosphonoalamide-like compounds produced by members of the phylum Bacillota. Cblaster, a tool used to rapidly identify loci of co-localized query genes within a genomic database, was used to find bacteria with the genetic potential to produce phosphonoalanine [11]. The two-gene cassette of pepM and aspC was detected in the genera Bacillus, Clostridia, Oscillobacter, Paenibacillus, Abyssisolibacter, Alicyclobacillus, and Oscillospiraceae. Most Bacilli, including numerous strains of Bacillus velezensis, encode a biosynthetic gene cluster identical to that observed in Mycobacteroides abscessus subsp. massilience. Narrowing the search to strains available from culture collections, Bacillus velezensis NRRL B-41580, Bacillus subtilis NRRL B-4247, Bacillus swezeyi NRRL B-41282, and Bacillus swezeyi NRRL B-41294 were obtained. With strains in hand, it was sought to determine culture conditions optimal for the production of phosphonates.

TABLE 1
Annotation of the B. velezensis NRRL B-41580 phosphonoalamide biosynthetic gene
cluster. Top NCBI BLASTp hits were produced by the direct alignment of M. abscessus
(FVSM01000012.1) and B. velezensis (NZ_LLZC01000022.1). B. velezensis protein families
were identified via Pfam.

| | | | Percent identity to | |
|---|---|---|---|---|
| | M. abscessus | M. abscessus NCBI PGAP | B. velezensis | B. velezensis Pfam |
| ORF | Accession | Annotation | (Accession) | (Accession) |
| orf1 | SLC11717.1 | putative secreted protein | 99.24% | protein of unknown |
| | | | (WP_053285257.1) | function DUF58 |
| | | | | (PF01882.20) |
| orf2 | SLC11728.1 | uncharacterized protein | 99.86% | transglutaminase-like |
| | | involved in cytokinesis | (WP_053285256.1) | superfamily |
| | | | | (PF01841.21) |
| orf3 | SLC11771.1 | GMP synthase | 100.00% | glutamine |
| | | | (WP_014417088.1) | amidotransferase |
| | | | | class-I (PF00117.30) |
| orf4 | SLC11800.1 | biotin carboxylase | 99.52% | ATP-grasp domain |
| | | | (WP_053285255.1) | (PF13535.8) |
| orf5 | SLC11817.1 | biotin carboxylase | 99.50% | ATP-grasp domain |
| | | | (WP_053285254.1) | (PF13535.8) |
| orf6 | SLC11840.1 | isocitrate lyase family | 98.98% | phosphoenolpyruvate |
| | | protein | (WP_053285253.1) | phosphomutase |
| | | | | (PF13714.8) |
| orf7 | SLC11886.1 | aspartate/tyrosine/aromatic | 99.50% | aminotransferase class |
| | | aminotransferase | (WP_053285252.1) | I and II (PF00155.23) |
| orf8 | SLC11928.1 | ABC transporter permease | 97.01% | major facilitator |
| | | | (WP_053285251.1) | superfamily |
| | | | | (PF07690.18) |
| orf9 | SLC11953.1 | RNA polymerase sigma | 98.87% | sigma-70, region 4 |
| | | factor | (WP_014417094.1) | (PF08281.14) |
| orf10 | SLC11990.1 | uncharacterized protein | 99.17% | domain of unknown |
| | | | (WP_053285250.1) | function (PF13786.8) |
| orf11 | SLC12013.1 | putative | 99.77% | permease family |
| | | hypoxanthine/guanine | (WP_007408891.1) | (PF00860.22) |
| | | permease | | |
| orf12 | SLC12048.1 | uncharacterized protein | 98.85% | no hits found |
| | | | (WP_053285249.1) | |

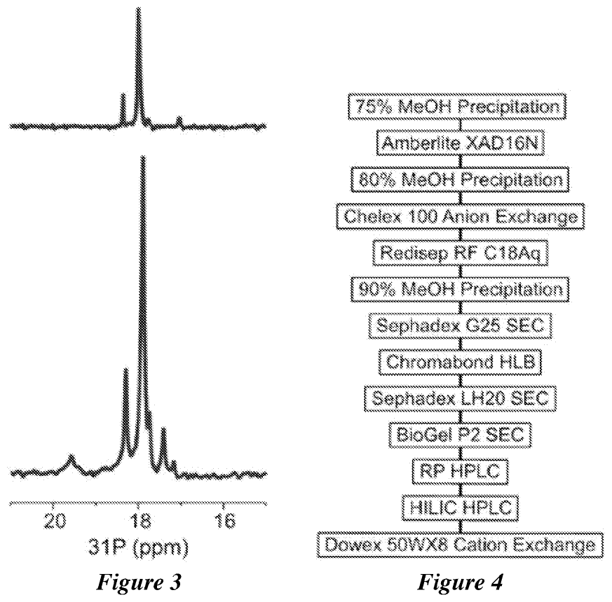

Phosphonate production screening and purification. FIG. 3-FIG. 7 illustrates the purification and structure elucidation of phosphonoalanine, phosphonoalamide E, and phosphonoalamide F.

Bacillus subtilis NRRL B-4257, Bacillus velezensis NRRL B-41580, Bacillus swezeyi NRRL B-41282, and Bacillus swezeyi NRRL B-41294 were grown in several different media types to determine optimal conditions for phosphonate production. Strains were grown in nutrient broth (NB), rhizocticin medium (RM), R2A medium with succinate (R2AS), GUBC medium (GUBC), and tryptic soy broth (TSB) (FIG. 3, top). Culture supernatants were concentrated under vacuum and screened for phosphonates by ³¹P NMR. All strains produced phosphonates in all media types except RM. Notably, Bacillus velezensis NRRL B-41580 produced phosphonates in the greatest abundance in all media types, but also produced the greatest number of distinct phosphonates in TSB. The results of this media screen prompted a phosphonate production timecourse experiment with Bacillus velezensis NRRL B-41580. Cultures were grown in TSB for 3, 5, and 7 days, and harvested via centrifugation. Culture supernatants were concentrated and screened for phosphonates as described above. Six distinct phosphonate peaks were observed on day 7, the greatest of all those tested, albeit several were present in very low abundance (FIG. 3, bottom). These findings provided the basis for culture scale-up to allow phosphonate purification (FIG. 4). Thirteen 1 L TSB cultures were inoculated and incubated for 7 days on a rotary shaker. Spent medium was obtained by centrifugation, and the cell pellet was further extracted with methanol. Aqueous and methanolic extracts were combined and concentrated to 1 L.

During the purification, all chromatography fractions were analyzed by ³¹P NMR, an invaluable tool in the isolation of phosphonate natural products. Methanol (75% v/v) was added to the crude extract to facilitate the precipitation of unwanted compounds. Precipitant was removed via centrifugation, and methanol was removed by rotary evaporation. Amberlite XAD16 resin was added to the concentrated methanol soluble fraction and the sample was gently agitated overnight. The unbound fraction was recovered by filtration, and the resin was washed with water to elute loosely bound material. The unbound and water fractions were combined, concentrated, and subjected to another methanol precipitation (80% v/v) as described above. The concentrated MeOH soluble fraction was further purified by anion exchange chromatography. The sample was acidified to pH 3 to selectively bind phosphonic acids, then applied to a bed of Fe(III)-chelated Chelex 100 resin in a glass column. The flow through was collected, and loosely bound material was eluted with acetic acid. The resin was then washed with increasing concentrations of base (500 mM NH₄HCO₃, 0.1% NH₄OH). Fractions containing phosphonates were combined and concentrated.

The sample was then subjected to reversed phase flash chromatography using a CombiFlash Rf+ instrument equipped with a RediSep Rf Gold C18Aq column. Following injection in water, the sample was eluted with increasing concentrations of acetonitrile in a gradient method. Fractions containing phosphonates were combined and concentrated, then precipitated with MeOH (90% v/v) as described above. The concentrated MeOH soluble fraction was further purified by size exclusion chromatography. The sample was fractionated over a bed of Sephadex G25 resin in a glass column, and fractions containing phosphonates were combined and lyophilized. Dry material was dissolved in water and applied to a bed of Chromabond HLB resin in a glass column. The flow through was collected, and then the sample was eluted with increasing concentrations of methanol in a stepwise gradient. The flowthrough, containing all phosphonates, was subjected to size exclusion chromatography twice more, first over Sephadex LH-20 resin, and then over BioGel P2 resin. BioGel P2 fractions containing phosphonates were pooled and lyophilized. The sample was then dissolved in water for reversed phase high performance liquid chromatography (RP-HPLC).

The sample was separated on a Phenomenex Fusion-RP column with a gradient elution program using water and methanol. Unfortunately, no phosphonates were retained on this column, which indicated a high degree of hydrophilicity. This prompted purification by hydrophilic interaction liquid chromatography (HILIC). The sample was dissolved in 70% acetonitrile, injected onto a Waters BEH Amide column, and eluted using a gradient method. This column retained and partially resolved all phosphonates but had to be repeated using an isochratic method (75% acetonitrile) to finish the purification. This column yielded 25 mg of pure compound 3, but still failed to resolve compounds 1 and 2. This sample was lyophilized, dissolved in water, and subjected to strong cation exchange chromatography. The sample was acidified to pH 3 before being applied to a bed of Dowex 50WX8 resin in a glass column. The flowthrough was collected, and then the resin was eluted with water followed by 0.1% formic acid. Water fractions were combined to yield 0.8 mg of compound 1, and formic acid fractions were combined to yield 1.1 mg of compound 2.

Structure Elucidation. Analysis of purified compound 1 by HRMS showed an [M+H]⁺ molecular ion of m/z 170.0212. The molecular formula C₃H₉NO₅P⁺ was deduced from this monoisotopic mass. Notably, this is the molecular formula of phosphonoalanine, an expected biosynthetic intermediate of phosphonoalamide-like peptides. Indeed, ¹H and ³¹P NMR data were identical to those of a synthetic standard, as well as literature values, thus identifying compound 1 as phosphonoalanine [9].

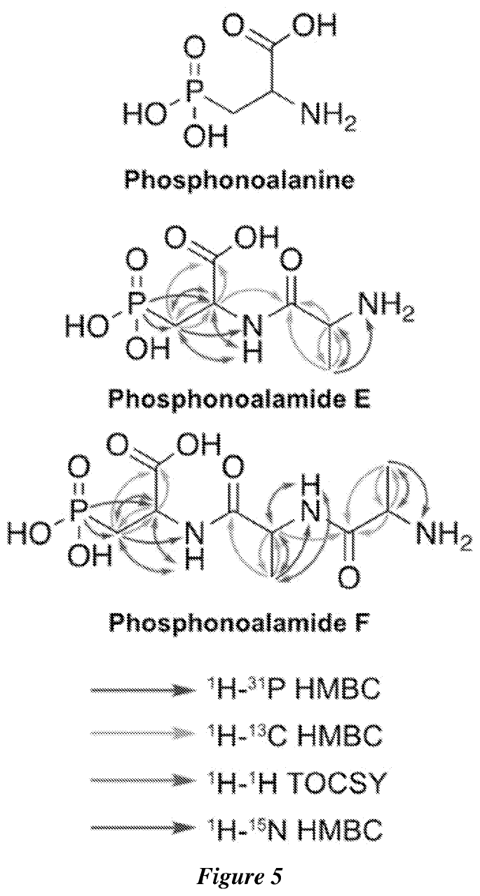

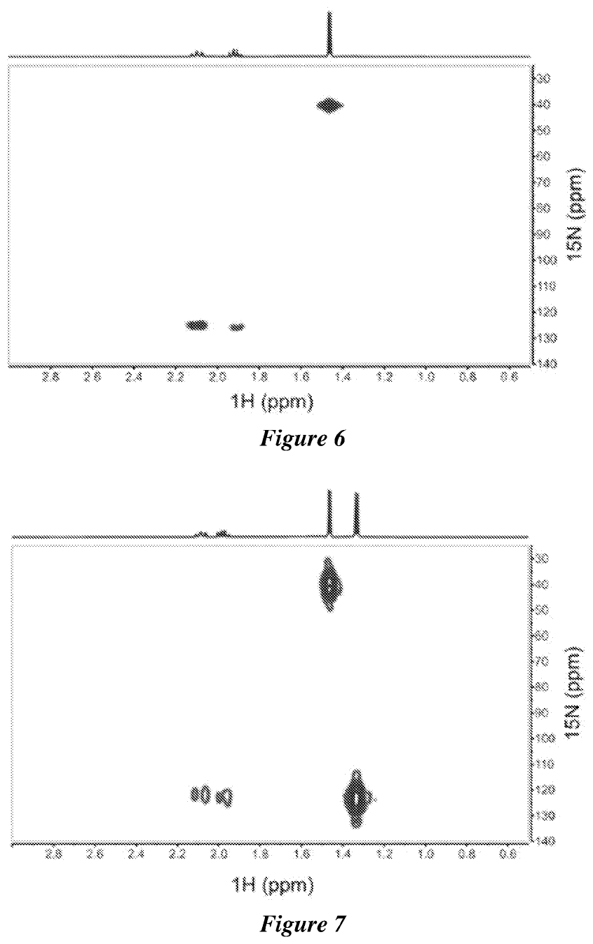

A high-resolution mass spectrum of compound 2 showed an |M+H|⁺ molecular ion of m/z 241.0583, from which the molecular formula C₆H₁₄N₂O₆P⁺ was determined. A suite of 1D and 2D NMR experiments were used to elucidate the chemical structure of this molecule (FIG. 5). The ¹H spectrum showed resonances for 8 protons, which a multiplicity-edited ¹H-¹³C HSQC experiment revealed to be a set of methyl protons (H-3′; δ_{H} 1.46), a set of methylene protons (H-1; δ_{H} 1.93, 2.11), and 2 methine protons (H-2′, H-2; δ_{H} 4.04, 4.35). The final resonance (H-b; δ_{H} 8.27) showed no correlations to a carbon atom, but a ¹H-¹⁵N HSQC experiment made evident a correlation to a nitrogen atom putatively involved in an amide bond (N-b; δ_{N} 124.96). In the ¹H-³¹P HMBC spectrum, a phosphorous atom was correlated to H-1 and H-2, indicating the presence of a P—C bond. This was further confirmed by the J_{CP} couplings of C-1 (δ_{C} 29.71), C-2 (δ_{C} 51.14), and C-3 (δ_{C} 177.18) in the ¹³C spectrum. The ¹H-¹H COSY and TOCSY spectra show that H-2 is adjacent to H-1 and H-b. Collectively, these 2D correlations indicated the presence of a phosphonoalanine residue. The amine (N-b) of phosphonoalanine participating in an amide bond places this residue at the C-terminus of the peptide (FIG. 6-FIG. 7). Notably, all phosphonoalanine-containing peptides discovered to-date are N-terminal phosphonopeptides [9].

The molecular formula of compound 2, and the presence of a phosphonoalanine residue, indicated that C₃H NO remained unassigned. An additional quaternary carbon (C-1′; δ_{C} 170.20), and the presence of an isolated spin system consistent with that of alanine in the ¹H-¹H COSY and TOCSY spectra, was suggestive of a PnAla-Ala dipeptide. Placement of the amide bond was confirmed by a correlation between H-2 and C-1′ in the ¹H-¹³C HMBC spectrum. Furthermore, a correlation between the methyl group of alanine (H-3′; δ_{H} 1.48) and the amine of alanine (N-c; δ_{N} 40.60) in the ¹H-¹⁵N HMBC spectrum confirmed that alanine is the N-terminal residue of this peptide. This is the first C-terminal phosphonoalanine-containing dipeptide reported to-date, which is named phosphonoalamide E herein.

Compound 3 gave an [M+H]⁺ molecular ion of m/z 312.0952 when analyzed by HRMS, which is in agreement with the molecular formula C₉H₁₉N₃O₇P⁺. The ¹H spectrum showed resonances for 13 protons, which a multiplicity edited ¹H-¹³C HSQC experiment ultimately proved to be 2 sets of methyl protons (H-3′, H-3″; δ_{H} 1.33, 1.46), a set of methylene protons (H-1; δ_{H} 1.98, 2.09), and 3 methine protons (H-2″, H-2′, H-2; δ_{H} 4.00, 4.29, 4.37). The final two resonances (H-b, H-c; δ_{H} 8.23, 8.57) showed one-bond correlations not to carbon atoms, but to nitrogen atoms (N-b, N-c; δ_{N} 122.00, 123.49) in the ¹H-¹⁵N HSQC spectrum. The chemical shift of these nitrogen atoms and their respective protons suggested they were involved in amide bonds. The ¹H-³¹P HMBC spectrum showed correlations nearly identical to those observed in phosphonoalamide E, with H-1 and H-2 correlated to a phosphorous atom (P-a; δ_{P} 19.13). The large J_{CP} coupling of C-1 indicates a direct bond to the phosphorous atom, while the splitting of C-2 and C-3 indicate they are within a 3-bond distance. The ¹H-¹H COSY and TOCSY spectra show that H-2 is adjacent to H-1 and amide H-b. These 2D correlations indicate that compound 2 also contains a phosphonoalanine residue with additional constituents attached at N-b.

The presence of two additional quaternary carbons (C-1″, C-1′; δ_{C} 170.69, 174.17), two amide protons (H-b, H-c), and two methine protons (H-2″, H-2′; δ_{H} 4.00, 4.29) within the range of alpha protons of amino acids indicated that phosphonoalanine resided at the C-terminus of a tripeptide. Two isolated spin systems in the ¹H-¹H COSY and TOCSY spectra, identical to that observed in phosphonoalamide E, show that compound 3 contains two alanine residues.

Placement of the Ala-Ala amide bond was confirmed by a correlation between H-2′ and C-1″ in the ¹H-¹³C HMBC spectrum. A correlation between the methyl group of the terminal alanine (H-3″; δ_{H} 1.46) and the amine of the terminal alanine (N-d; δ_{N} 40.22) further confirmed that an alanine residue resides at the N-terminus of the tripeptide (FIG. 6-FIG. 7). This N-Ala-Ala-PnAla-C peptide is named phosphonoalamide F herein.

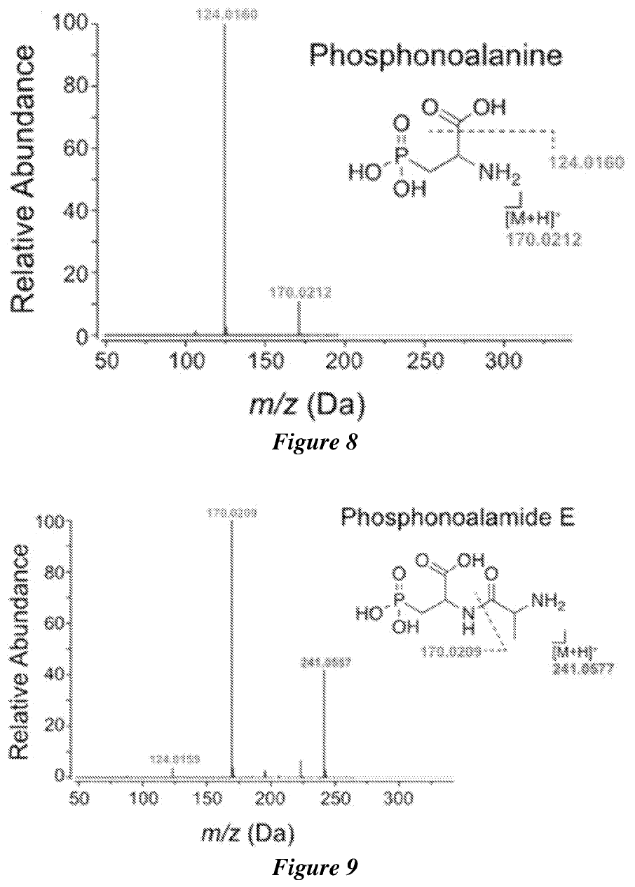

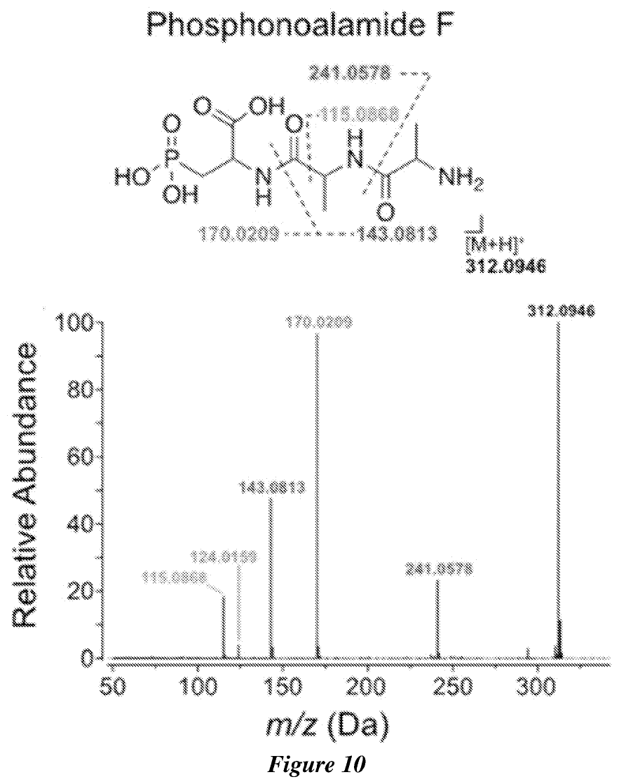

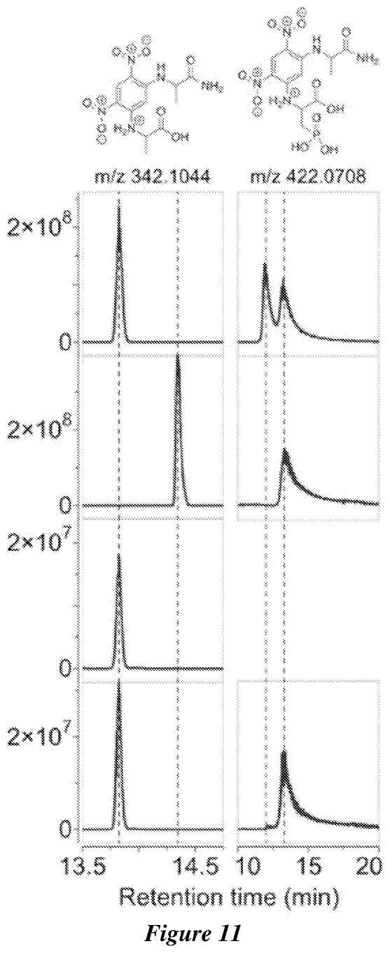

Tandem MS data corroborated the proposed structures of all three compounds isolated in this study (FIG. 8-FIG. 10). A PnAla-Ala dipeptide was observed in the spectra of phosphonoalamide F (fragment) and phosphonoalamide E (parent). Further, phosphonoalanine was observed in the spectra of phosphonoalamide F (fragment), phosphonoalamide E (fragment), and phosphonoalanine (parent). Finally, a decarboxylated fragment of phosphonoalanine was observed in the spectra of all compounds. To determine amino acid stereochemistry, peptides were acid hydrolyzed, derivatized with Marfey's reagent, and subjected to RP-HPLC (FIG. 11). Phosphonoalamide E and F were found to contain L-alanine and L-phosphonoalanine.

Biological Activity. Phosphonoalamide E and F were evaluated for antimicrobial activity against a panel of bacteria, yeast, and filamentous fungi in a combination of microbroth dilution and disk diffusion assays. Both compounds showed antibacterial activity, with greatest potency against several strains of Bacillus (Table 2), notably belonging to the same genus as the producing organism. In the context of chemical ecology, microbes capable of producing compounds which inhibit the growth competitors in their niche certainly hold an evolutionary advantage. This may partially explain the spectrum of activity of these Bacillus derived phosphonopeptides. Interestingly, phosphonoalamide E was less inhibitory towards each of these strains, and did not inhibit any other genera. This may be due to a differential in cellular uptake, speaking to the selectivity of oligopeptide permeases in strains that were inhibited by phosphonoalamide F, but not phosphonoalamide E.

Phosphonoalamide F was also active against several Gram-negative bacteria, including Escherichia coli K12, Pseudomonas aeruginosa K, Salmonella enterica LT2, and Serratia marcescens NRRL B-2544. Intriguingly, phosphonoalamide F was inactive against Escherichia coli WM6242, a strain which encodes IPTG-inducible phosphonate uptake transporters. This result indicates a lack of uptake by such transporters, but does not explain strain selectivity in the inhibition of E. coli. P. aeruginosa, S. enterica, S. marcescens, and E. coli are human pathogens, and further investigating the mechanism of action of the phosphonoalamides may reveal drug targets.

TABLE 2
MIC90 values of phosphonalamide E and F against a panel of bacteria in microbroth
dilution assays. All assays were done in triplicate as independent experiments.

| Organism | MIC (μM) | |
|---|---|---|
| Order/Strain | Phosphonoalamide E | Phosphonoalamide F |
| Enterobacterales | | |
| Citrobacter freundii ATCC 8090 | >200 | >200 |
| Enterobacter aerogenes CDC 1998-68 | >200 | >200 |
| Escherichia coli K12 | >200 | 6.25 |
| Escherichia coli WM6242 | >200 | >200 |
| Escherichia coli WM6242 (Pn Uptake | >200 | >200 |
| Induced) | | |
| Salmonella enterica LT2 | >200 | 12.5 |
| Serratia marcescens NRRL B-2544 | >200 | 200 |
| Pantoea ananatis LMG 20103 | 100 | 3.12 |
| Erwinia rhapontici KSJ | >200 | 6.25 |
| Actinomycetales | | |
| Rhodococcus jostii RHA1 | >200 | >200 |
| Bacillales | | |
| Staphylococcus aureus ATCC 23055 | >200 | >200 |
| Bacillus subtilis ATCC 6633 | 200 | 12.5 |
| Bacillus megaterium ATCC 19213 | 12.5 | 6.25 |
| Pseudomonadales | | |
| Acinetobacter calcoaceticus ATCC 23055 | >200 | >200 |
| Pseudomonas aeruginosa K | >200 | 25 |
| Pseudomonas aeruginosa PA01 | >200 | >200 |

MIC = Minimum inhibitory concentration

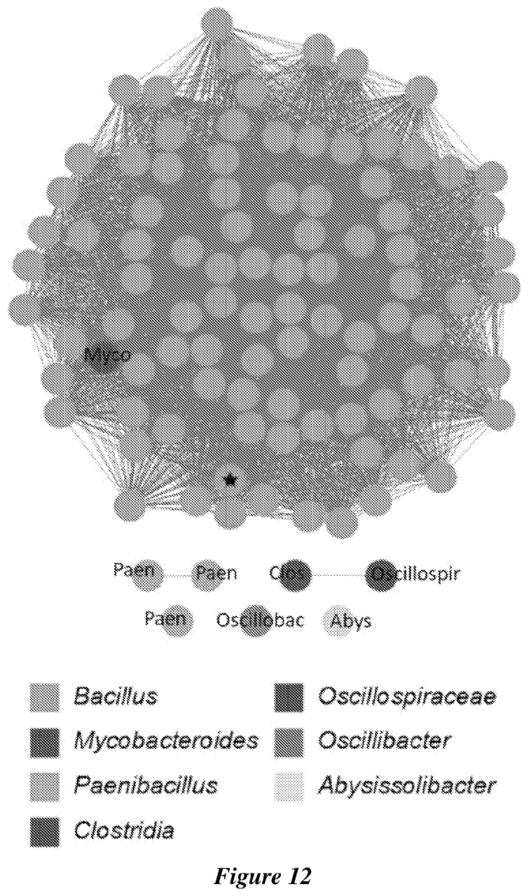

Diversity of Phosphonoalanine biosynthetic gene clusters in Bacillota. The majority of microbes in the phylum Bacillota capable of producing phosphonoalanine encode a phosphonate biosynthetic gene cluster nearly identical to that of Bacillus velezensis NRRL B-41580, and likely produce phosphonoalamide derivatives. These biosynthetic gene clusters contain two ATP-grasp ligases, suggesting the production of tripeptides, although there is now precedent for the formation of dipeptides. In a sequence similarity network of PepM (FIG. 12), sequence diversity appears to be positively correlated to biosynthetic gene cluster diversity, as has been reported previously [5]. Interestingly, strains of Paenibacillus and Oscillibacter appear capable of producing only free phosphonoalanine. This is also the case for Clostridia bacterium RGIG2164 and Oscillospiraceae bacterium BX1, but they encode an amino acid racemase downstream of pepM. If phosphonoalanine is indeed the inhibitory payload of the phosphonoalamides, this may be a mechanism of self-resistance, as a racemic mixture of phosphonoalanine was previously shown to be significantly less potent than enantiopure L-phosphonoalanine [9]. Lastly, compared to known phosphonoalamide producers, Abyssisolibacter fermentans MCWD3 encodes several additional enzymes with potential to ligate amino acids to phosphonoalanine. This may be representative of a pathway to larger tetra- or penta-phosphonopeptides.

DISCUSSION The discovery of phosphonoalamides E-F from Bacillus velezensis expands the knowledge of phosphonate metabolism in taxonomically diverse microbes. Until now, only one other member of the genus Bacillus was known to produce phosphonates, despite the widespread distribution of pepM in this taxon. Unlike the strictly antifungal rhizocticins produced by Bacillus subtilis, phosphonoalamides E-F show broad spectrum antibacterial activity. Potent inhibition of the human pathogens E. coli, S. enterica, and P. aeruginosa highlights their potential for antibiotic development. Further, investigating their mechanism of action may reveal novel drug targets, and enable the development of more potent analogs through synthetic biology or medicinal chemistry efforts.

Notably, the potential applications of Bacillus derived phosphonoalamides are not limited to pharmaceutical development. These compounds also inhibited the plant pathogens P. ananatis and E. rhapontici, demonstrating their potential for development as pesticides. P. ananatis is a pathogen of numerous cash crops, including maize, rice, tomato and melons, but is most burdensome in onion agriculture [12]. As the causative agent of onion center rot, P. ananatis can cause crop losses of up to 100% [13]. In total, bacterial pathogens cause $60 million dollars in onion crop losses annually, and a lack of bactericides limits the industries' capacity to mitigate these losses [14]. Microbial natural products active against P. ananatis, such as phosphonoalamides E-F, offer promise as pesticides which could be used prophylactically or at the onset of infection in the field, as well as during bulb storage prior to sale. Erwinia rhapontici is the causative agent of rhubarb crown rot, also known as red leaf or bacterial soft rot. This bacterium can also cause rot in peas, cucumbers, and onions [14]. Altogether, these findings suggest that purified phosphonoalamides E-F, crude culture extracts, or even live cultures of Bacillus velezensis NRRL B-41580 could be used to combat microbial pests in commercial agriculture.

To date, the large majority of phosphonate discovery efforts have focused on actinobacteria, due to their long track record of commercially successful secondary metabolites, particularly as drugs. However, advancements in next generation sequencing have shown that the potential for phosphonate biosynthesis well-distributed in taxonomically diverse microbes. Herein, the discovery of antimicrobial phosphonopeptides from Bacillus velezensis, which are active against both human and plant pathogens was detailed. These findings demonstrate the importance of investigating phosphonate metabolism in genera which are historically underexplored.

### Materials and Methods

Chemicals and Reagents. All chemicals and reagents were from Sigma-Aldrich, Fisher Scientific, or VWR unless otherwise indicated.

Strains, Media, and General Culture Conditions. Strains used in this study are listed in Table 3. Rhodococcus, Serratia, Bacillus, and fungal strains were grown at 30° C. All other strains were grown at 37° C.

Media used in this study include: nutrient broth (NB; 3 g beef extract, 5 g peptone), GUBC, R2AS, tryptic soy broth (TSB; 17 g tryptone, 3 g soytone, 2.5 g dextrose, 5 g NaCl, 2.5 g K2HPO₄; pH to 7.3 prior to autoclaving), rhizocticin media (RM), M9 with 20 mM glucose, YPD (10 g yeast extract, 20 g peptone, 20 g dextrose), yeast minimal media (YMM; 6.7 g yeast nitrogen base without amino acids, 20 g dextrose), malt extract media (MEM; 20 g malt extract, 20 g dextrose, 6 g peptone). M9 glucose medium was supplemented with 1 mM thiamine-HCl and 0.05 mM nicotinamide for Staphylococcus (SSM9PR medium). All components were dissolved in deionized water (dI H₂O). 16 g of agar was added per liter of media.

TABLE 3
Strains used in bioassays
Strains
Enterobacterales
Citrobacter freundii ATCC 8090
Enterobacter aerogenes CDC 1998-68
Escherichia coli K12
Escherichia coli WM6242
Salmonella enterica LT2
Serratia marcescens NRRL B-2544
Pantoea ananatis LMG 20103
Erwinia rhapontici KSJ
Actinomycetales
Rhodococcus jostii RHA1
Mycobacterium smegmatis NRRL B-14616
Bacillales
Staphylococcus aureus ATCC 23055
Bacillus subtilis 168
Bacillus subtilis ATCC 6633
Bacillus cereus ATCC 14579
Bacillus megaterium ATCC 19213
Pseudomonadales
Acinetobacter calcoaceticus ATCC 23055
Pseudomonas aeruginosa K
Pseudomonas aeruginosa PA01
Yeasts
Candida albicans ATCC MYA-2876
Debaryomyces hansenii CBS-767
Saccharomyces cerevisiae KSJ4150
Schizosaccharomyces pombe ATCC 24843
Kluyveromyces lactis Y-8279
Sporobolomyces roseus KSJ4237
Filamentous Fungi
Aspergillus niger ATCC 16404
Aspergillus nidulans FGSC A4
Neurospora crassa FGSC 4200
Penicillium notatum ATCC 9478
Penicillium commune KSJ4156

Bioinformatics. Sequences encoding putative biosynthetic gene clusters were retrieved from NCBL Open reading frames and synteny were analyzed using EasyFig, clinker, and cblaster. BLAST analyses were performed against the NCBI non-redundant (nr) protein database and PFam.

NMR and Mass Spectrometry. All NMR and LC-MS data were analyzed using MestReNova software. ¹H, ¹³C, and ¹⁵N NMR spectra were acquired at 25° C. in either 10% D2O or DMSO-d6 on a Bruker Avance III HD 700 MHz spectrometer equipped with a triple resonance cryoprobe. ³¹P NMR spectra were recorded on a Bruker Avance III HD 600 MHZ spectrometer equipped with a 5 mm Smart Broadband Probe. HRMS and HRMS/MS were performed on an Agilent 6540 UHD Accurate-Mass Q-TOF system, equipped with an Agilent 1260 Infinity II HPLC

Production Screening. Bacillus velezensis NRRL B-4257, Bacillus velezensis NRRL B-41580, Bacillus swezeyi NRRL B-41282, and Bacillus swezeyi NRRL B-41294 were revived onto TSB plates from glycerol stocks and inoculated in 20×150 mm test tubes containing 5 mL of the same medium. Starter cultures were grown at 30° C. for 24 hours on a rotary shaker (220 rpm), used to inoculate 125 mL baffled flasks containing 25 mL of NB, GUBC, R2AS, TSB, or RM (500 μL each), and incubated on a rotary shaker (30° C., 220 rpm) for 3 days. Timecourse experiments were performed 2.5 L Ultra-Yield flasks containing 1 L of production media. These were inoculated with 10 mL of starter cultures and incubated on a rotary shaker (30° C., 220 rpm). Samples were withdrawn (25 mL) at 3, 5, and 7 days of growth.

All samples were centrifuged at 10,000 RPM, 4° C., for 10 minutes. Clarified supernatants were lyophilized and reconstituted in 1 mL of dI H₂O. Concentrated extracts then were amended with 10% D₂O for ³¹P NMR analysis. Putative phosphonic acids were identified as ³¹P NMR resonances with chemical shifts 8 ppm or greater.

Production Scale-Up. A starter culture of Bacillus veleznesis NRRL B-41580 (as above) was used to inoculate 500 mL Fernbach flasks containing 125 mL TSB medium. After 36 hours of growth at 30° C. on a rotary shaker (220 RPM), seed cultures were used to inoculate 13 individual 2.5 L Ultra-Yield flasks each containing 1 L of TSB. These production cultures were grown for 7 days at 30° C. on a rotary shaker (220 RPM). Cultures were harvested by centrifugation (10,000 RPM, 4° C., for 10 minutes). Clarified supernatants was removed and set aside. Cell pellets were resuspended in 500 mL methanol and vigorously agitated by vortexing to extract residual metabolites. The aqueous and methanolic fractions combined to yield 13.5 L of starting material.

Marfey's Analysis. Compounds 2-3 (0.2 mg) were dissolved in 0.5 mL of 6N HCl and heated to 100° C. in a sealed reaction vial for 16 hours. Samples were dried at 40° C. under a gentle stream of air to remove HCl. Hydrolysates were dissolved in 50 μL of water and transferred to microcentrifuge tubes, and 50 mM solutions (50 μL) of each amino acid standard were prepared. Each sample or standard was combined with 20 μL of 1M NaHCO₃ and 100 μL of a 1% solution of FDAA in acetone and incubated on a heating block at 40° C. for 1 hour. Samples were cooled to room temperature and neutralized with 20 μL of 1M HCl. Derivatized amino acid standards and hydrolysates were diluted 50-fold into 10% MeCN with 0.1% formic acid. Derivatized PnAla solutions were diluted 10-fold into 10% MeCN with 0.1% formic acid. These were analyzed by LC-MS using a Zorbax Extend-C¹⁸ column (2.1×50 mm, 1.8 μm) with a gradient of 10-60% MeCN with 0.1% formic acid over 20 min. The derivatized phosphonoalanine stereoisomers were both poorly retained and resolved on the above column, so compound 1 and standards were subjected to LC-MS analysis using on a Synergi Fusion-RP column (100×2 mm, 4 μm) using a gradient of 0-100% MeOH with 0.1% formic acid over 30 min.

Antimicrobial Assays. Susceptibility testing was performed using the microbroth dilution method in 96 round-well microtiter plates following the general guidelines described by the Clinical and Laboratory Standards Institute (CLSI). Compounds were prepared as 50λ of stocks (10 mM) by dissolving in dI H2O. All bacterial strains were grown and assayed in Mueller-Hinton and M9 glucose media (with amendments added as required), and yeast strains in YMM. Overnight cultures were inoculated into 3 mL of fresh growth media following a 1:100 dilution. Upon mid-exponential growth (OD600 0.4-0.6), cultures were diluted to 5×10⁶ CFU mL-1 (based on previously determined calibration curves) and used as inoculum into assay plates. Wells contained 100 μL of media with 5×105 CFU mL-1 of test organism, and serial ½-dilutions of the test compound (starting with 200 μM). E. coli WM6242 was assayed with induction of the phosphonic acid uptake system including 1 mM IPTG in assay wells. Controls wells contained 200 UM kanamycin (for bacteria) or nystatin (for yeasts), no compound addition (vehicle only), and no cell addition (added media instead). Microtiter plates were incubated with shaking (220 rpm) at their respective growth temperatures. Culture densities (OD600) were recorded using a Bio-Rad xMark microplate spectrophotometer after 16 hours for all strains except Bacillus, which were after 24 hours. Assays were performed in triplicated on separate independent days. Minimum inhibitory concentration (MIC) values were defined as the lowest concentration of compound that resulted in ≥90% growth inhibition. Minimum inhibitory concentrations are from triplicate assays performed on separate days.

Disk diffusion assays were also used to assess antimicrobial activity of the purified compounds against filamentous fungi, and select bacterial strains. M9 glucose plates were inoculated with 50 μL of diluted stock cultures of Mycobacterium smegmatiss and Bacillus cereus from above. Filamentous fungi were grown in 3 mL starter cultures containing MEM for 3 days or M9 glucose for 5 days and then swabbed onto MEM and M9 glucose agar plates, respectively. 20 μL of 1 mM phosphonoalamide, 1 mM nystatin, or dI H2O was applied to 6 mm antibiotic assay filter disks (Whatman) and dried in a biosafety cabinet. Dried paper disks were placed in the center of each plate and incubated at 30° C. or 37° C. as appropriate. Bacterial plates were incubated for 24 hours, yeast plates 3 days, fungal MEM plates 3-5 days, and fungal M9 glucose plates 5-7 days.

### REFERENCES

(1) Metcalf W W et al. Biosynthesis of phosphonic and phosphinic acid natural products. Annu Rev Biochem 2009, 78, 65-94. DOI: 10.1146/annurev.biochem.78.091707.100215.
(2) Rodolfo, P. Antimicrobial resistance: time to repurpose the Global Fund. 2022.
(3) Ju K S et al. Genomics-enabled discovery of phosphonate natural products and their biosynthetic pathways. Journal of industrial microbiology & biotechnology 2014, 41 (2), 345-356.
(4) Yu X et al. Diversity and abundance of phosphonate biosynthetic genes in nature. Proc Natl Acad Sci USA 2013, 110 (51), 20759-20764. DOI: 10.1073/pnas.1315107110.
(5) Ju K S et al. Discovery of phosphonic acid natural products by mining the genomes of 10,000 actinomycetes. Proc Natl Acad Sci USA 2015, 112 (39), 12175-12180. DOI: 10.1073/pnas. 1500873112.
(6) Kugler M et al., Rhizocticin A, an antifungal phosphono-oligopeptide of Bacillus subtilis ATCC 6633: biological properties. Arch Microbiol 1990, 153 (3), 276-281. DOI: 10.1007/BF00249082.
(7) Laber B et al. Inactivation of Escherichia coli threonine synthase by D L-Z-2-amino-5-phosphono-3-pentenoic acid. Archives of microbiology 1994, 161 (5), 400-403.
(8) Polidore A L A et al. A phosphonate natural product made by Pantoea ananatis is necessary and sufficient for the hallmark lesions of onion center rot. Mbio 2021, 12 (1), e03402-03420.
(9) Kayrouz C M et al. Genome Mining Reveals the Phosphonoalamide Natural Products and a New Route in Phosphonic Acid Biosynthesis. ACS Chemical Biology 2020, 15 (7), 1921-1929.
(10) Bowman E et al. Catalysis and thermodynamics of the phosphoenolpyruvate/phosphonopyruvate rearrangement. Entry into the phosphonate class of naturally occurring organophosphorus compounds. Journal of the American Chemical Society 1988, 110 (16), 5575-5576.
(11) Gilchrist C L et al. cblaster: a remote search tool for rapid identification and visualization of homologous gene clusters. Bioinformatics Advances 2021, 1 (1), vbab016.
(12) Coutinho T A et al. Pantoea ananatis: an unconventional plant pathogen. Molecular plant pathology 2009, 10 (3), 325-335.
(13) Gitaitis R D et al. First report of a leaf blight, seed stalk rot, and bulb decay of onion by Pantoea ananas in Georgia. Plant disease 1997, 81 (9), 1096-1096.
(14) Huang H C et al. Biology and epidemiology of Erwinia rhapontici, causal agent of pink seed and crown rot of plants. [symbol] [symbol] 2003.

### Example 2—Discovery of Antimicrobial Phosphonopeptide Natural Products from Bacillus velezensis by Genome Mining

ABSTRACT. Phosphonate natural products are renowned for inhibitory activities which underly their development as antibiotics and pesticides. Although most phosphonate natural products have been isolated from Actinobacteria, bioinformatic surveys suggest many other bacterial phyla are replete with phosphonate biosynthetic potential. While mining actinobacterial genomes, a contaminated Mycobacteroides dataset was encountered which encoded a biosynthetic gene cluster predicted to produce novel phosphonate compounds. Sequence deconvolution revealed that the contig containing this cluster, as well as many others, belonged to a contaminating Bacillus, and are broadly conserved among multiple species, including the epiphyte B. velezensis. Isolation and structure elucidation revealed a new di- and tripeptide composed of L-alanine and a C-terminal L-phosphonoalanine which were named phosphonoalamide E and F herein. These compounds display broad-spectrum antibacterial activity, with strong inhibition against the agricultural pests responsible for vegetable soft rot (Erwinia rhapontici), onion rot (Pantoea ananatis), and American foulbrood (Paenibacillus larvae). This work expands the knowledge of phosphonate metabolism and underscores the importance of including underexplored microbial taxa in natural product discovery.

Phosphonate natural products produced by bacteria have been a rich source of clinical antibiotics and commercial pesticides. In this study, the discovery of two new phosphonopeptides produced by B. velezensis with antibacterial activity against human and plant pathogens, including those responsible for widespread soft rot in crops and American foulbrood, are described. The results shed new insight on the natural chemical diversity phosphonates and suggest these compounds could be developed as effective antibiotics for use in medicine and or agriculture.

INTRODUCTION. Phosphonate and phosphinate (collectively Pn) natural products (NPs) are a class of clinically and industrially used compounds due to their potent bioactivities. Their defining carbon to phosphorous (C—P) bond provides resistance to degradation, while isosterism of the phosphonate/phosphinate moiety to phosphate and carboxylate functional groups enables highly specific competitive or suicide inhibition of essential metabolic enzymes. Primary metabolites containing carboxylate, phosphate ester, and phosphoanhydride groups can be mimicked by phosphonate/phosphinate inhibitors. Indeed, several phosphonate/phosphinate natural products have been commercialized as essential research reagents, antibiotics, and herbicides. Fosmidomycin and FR-900098, respectively isolated from Streptomyces lavendulae and S. rubellomurinus, both inhibit 1-deoxy-D-xylulose 5-phosphate reductoisomerase (DXR) to block the Rohmer pathway of isoprenoid biosynthesis [A1-A4]. While essential in plant chloroplasts, malarial parasites, and many bacterial pathogens, the absence of this pathway in humans has prompted exploration of both compounds as potential clinical therapies [A5, A6]. Fosfomycin was originally discovered from several strains of Streptomyces and later again in Pseudomonas [A7, A8]. Prescribed for bacterial cystitis, fosfomycin forms a covalent adduct with UDP-N-acetylglucosamine enolpyruvyl transferase to block peptidoglycan synthesis [A9]. Lastly, phosphinothricin (PT) and its peptide conjugants (both originally isolated from Streptomyces) are potent inhibitors of glutamine synthetase and are the active ingredients within several lines of commercial and industrial herbicides produced by BASF [A10]. These examples demonstrate the broad importance of phosphonate/phosphinate natural products in medicine, agriculture, and biotechnology.

Molecular studies have elucidated the biosynthetic pathways for many phosphonate/phosphinate natural products, revealing a wealth of unusual enzymology and new metabolic paradigms [A11, A12]. Collectively, these studies have provided genetic and biochemical markers to classify strains by phosphonate/phosphinate production and the bioinformatic prediction of their intermediates and end-products. Perhaps most importantly, all genetically characterized pathways for phosphonates/phosphinates initiate with the isomerization of phosphoenolpyruvate (PEP) to phosphonopyruvate (PnPy) by phosphoenolpyruvate mutase (PepM) [A13-A15]. Based on this universally conserved first step, 6% of microbes encode biosynthetic gene clusters (BGCs) containing pepM, of which only a minuscule fraction have been characterized [A16]. The wealth of undiscovered phosphonate/phosphinate natural products suggested by these surveys, combined with their historically high rate of commercialization and efficacy [A17], have renewed attention on these molecules as a source of novel pharmacophores in drug and herbicide development.

Molecular, genomic, bioinformatic, and analytical methods have been developed to empower access to new phosphonate/phosphinate natural product by genome mining [A16, A18, A19]. Targeted discovery of phosphonates/phosphinates from cryptic (uncharacterized) biosynthetic gene clusters has uncovered new herbicidal and antimicrobial compounds, previously undescribed natural product scaffolds, and the pathways and enzymes underlying their biosynthesis |A18-A23|. These studies have also revised the understanding of the origin and function for many phosphonates/phosphinates, uncovering new roles for compounds previously thought to exist solely as components of macromolecules, metabolic side-products, or chemical synthons. Aminomethylphosphonate (AMPn), a component of synthetic drugs, commercial herbicides, and industrial anti-scaling agents, was recently found as a component of argolaphos A and B, broad spectrum antibacterial phosphonopeptides produced by multiple strains of Streptomyces [A18]. The presence of the aminomethylphosphonate biosynthesis genes among diverse microbes suggests its incorporation in many other inhibitory natural products [A22]. Given the ubiquity of 2-aminoethylphosphonate (2AEPn) as a headgroup of lipids, glycans, and glycoproteins within in bacteria, microbial eukarya, and mammals, it has been estimated to be the most abundant phosphonate/phosphinate natural product in nature [A24]. With the recent discoveries of the phosphonocystoximate natural products [A18] and fosmidomycin biosynthetic pathway [A25], there is growing recognition of 2-aminoethylphosphonate as an important core scaffold of small-molecule phosphonates/phosphinates.

Genome mining has also revised the understanding of phosphonoalanine (PnAla), a non-canonical phosphonate amino acid best known as a potent agonist of glutamate receptors [A26] and as a common component of macromolecular lipids in microbial Eukarya [A27]. While microbial degradation of phosphonoalanine has been well characterized [A28, A29], its biosynthesis in bacteria has only recently come into light. The bacterial pathway for phosphonoalanine was discovered by targeting phosphonates/phosphinates biosynthetic gene clusters that lacked genes encoding for established phosphoenolpyruvate mutase-coupling enzymes within Streptomyces. Isolation of the phosphonates/phosphinates led to the discovery of the phosphonoalamides, a series of antibacterial peptides containing an N-terminal phosphonoalanine residue [A20]. Heterologous expression established that only pepM and a homolog of aspartate transaminase (putative phosphonopyruvate-transaminase) were required to transform phosphoenolpyruvate into phosphonoalanine (via phosphonopyruvate). Both genes were present within genetically diverse biosynthetic gene clusters, suggesting phosphonoalanine biosynthesis as a new branchpoint in phosphonate/phosphinate metabolism and a common strategy towards the synthesis of inhibitory phosphonate/phosphinate natural products [A20].

In this study, a series of phosphonate/phosphinate natural products produced by phosphonoalanine biosynthetic pathways in bacteria are reported. Guided by genome mining, a survey of publicly available genomes suggested additional pathways for phosphonoalanine natural products from actinobacteria, including Mycobacteria. Upon further analysis, it was determined that this phosphonate/phosphinate biosynthetic gene cluster was not of mycobacterial origin, but rather from Bacillus velezensis. Purification and structure elucidation revealed phosphonopeptides with phosphonoalanine at the carboxyl-terminus. These compounds exhibited strong inhibitory activity against plant and animal bacterial pathogens. The conservation of this biosynthetic gene cluster within numerous strains and species of plant-associated Bacillus suggests these compounds may have a role in modulating microbial community composition within these environments.

### Results

Identification of a Bacillus phosphonoalanine biosynthetic gene cluster from a contaminated actinobacterial dataset. Initial analyses were focused on actinobacteria, which contains the greatest overall diversity of unstudied phosphonate/phosphinate natural product biosynthetic gene clusters in bacteria [A16]. First, all actinobacterial genomes within NCBI that contained pepM were identified. The resulting 20-kb neighborhoods surrounding pepM were then screened for the presence of the putative phosphonoalanine aminotransferase [A20] and absence of phosphonopyruvate decarboxylase (PPD) [A30, A31], phosphonomethylmalate synthase (PMS) [A32], and phosphonopyruvate reductase (PPR) [A23]. These four enzymes serve to define the early branchpoints in phosphonate/phosphinate biosynthesis and provide the thermodynamic driving force necessary to overcome the unfavorable energetics of phosphonopyruvate formation. Based on these analyses, only 13 of the 592 actinobacterial phosphonate/phosphinate biosynthetic gene clusters were suggested to proceed through phosphonoalanine.

Based on a phosphoenolpyruvate mutase sequence identity of 80% or greater, a threshold that effectively delineates both similarity of phosphonate/phosphinate biosynthetic gene clusters and their resulting natural products [A16], the 13 phosphonoalanine-biosynthetic gene clusters were divided into 3 gene-cluster families (GCFs). Setting aside the 11 strains previously established to produce phosphonoalamide A-D [A20], the remaining 2 were separated into their own individual groups. Extensive genome fragmentation precluded meaningful neighborhood analysis of the first singleton pepM biosynthetic gene cluster (Streptomyces sp. SID7982). Interestingly, the remaining cryptic biosynthetic gene cluster originated from the genomic assembly of Mycobacteroides abscessus (formerly Mycobacterium abscessus) subsp. massilience strain aerosol_aerosol_3 (henceforth designated as AA3). While only a limited number of natural products have been discovered from mycobacteria, all have proven to be essential to their physiology and pathogenesis. These include siderophores such as the mycobactins, carboxymycobactins, and exochelins, and the essential co-factor mycothiol [A33, A34|. Based on this reasoning, it was hypothesized that the phosphonate/phosphinate natural product produced from this biosynthetic gene cluster may also provide similar advantages in Mycobacteroides pathogenesis, such as antagonistic properties useful for promoting its survival within human or animal hosts.

First, the AA3 assembly was analyzed to verify the source of the biosynthetic gene cluster and identify strains which could be readily obtained to characterize potential phosphonate/phosphinate natural products. Surprisingly, BLAST analysis of the pepM from AA3 matched not to mycobacteria, but to strains of Bacillus (ranging from 69-99% identity). As Mycobacteroides and Bacillus belong to taxonomically distinct phyla, it was hypothesized that the phosphonate/phosphinate biosynthetic gene cluster may have been derived from horizontal gene-transfer, or alternatively, indicative of contamination of the Mycobacteroides isolate or the sequencing process. To understand the nature of this result, the AA3 dataset was first compared to other closely related strains. Whereas mycobacterial and mycobacteroides genomes average 5.6±0.9 Mbps and 5.1±0.3 Mbps, respectively (Table 4 and Table 5) [A35, A36], the AA3 assembly was a clear outlier at 10.7 Mb. Comparative analysis of the 3,914 contigs binned them to six genera of bacteria. The overwhelming majority (64.1%; 2,510 contigs) were assigned to Bacillus, and the remainder of the contigs were distributed among Mycobacteroides (32.4%; 1,268 contigs), Staphylococcus (1.4%; 54 contigs), Rhodococcus (0.05%, 2 contigs), Rhodoferax (0.02%; 1 contigs), and Streptomyces (0.02%; 1 contigs) (FIG. 1). Forty-two contigs (1.2%) did not match to any known organisms. Considering total nucleotide content, 80.6% (8.6 Mbp) was attributed to Bacillus, 17.5% (1.9 Mbp) to Mycobacteroides, and all remaining genera each representing <1% of the total dataset. Three 16S rRNA genes were detected, matching to Bacillus velezensis Pilsner 2-2 (99.9% identity), Staphylococcus epidermidis NRC 113846 (100%), and Mycobacteroides abscessus T84b (100%). Thus, the AA3 assembly was a clearly a mixed dataset with DNA from multiple strains.

TABLE 4
Mycobacterium genomes analyzed in this study

| | | | Genome | |
|---|---|---|---|---|
| | | | Size | |
| Species | Strain | Assembly | (Mbp) | GC % |
| Mycobacterium stephanolepidis | NJB0901 | GCA_002356335.1 | 4.99 | 65.0 |
| Mycobacterium vulneris | DSM 45247 | GCA_000612885.1 | 6.98 | 66.7 |
| Mycobacterium lentiflavum | ATCC | GCA_022374895.1 | 6.16 | 65.9 |
| | 51985 | | | |
| Mycobacterium europaeum | DSM 45397 | GCA_002102155.1 | 5.63 | 68.5 |
| Mycobacterium interjectum | ATCC | GCA_900078675.2 | 5.93 | 67.6 |
| | 51457 | | | |
| Mycobacterium mungi | BM22813 | GCA_001652545.1 | 4.35 | 65.5 |
| Mycobacterium szulgai | DSM 44166 | GCA_002116635.1 | 6.67 | 65.8 |
| Mycobacterium scrofulaceum | DSM 43992 | GCA_002086735.1 | 6.17 | 68.4 |
| Mycobacterium intermedium | DSM 44049 | GCA_002086275.1 | 6.82 | 65.8 |
| Mycobacterium sherrisii | ATCC | GCA_002102355.1 | 5.56 | 67.0 |
| | BAA-832 | | | |
| Mycobacterium shimoidei | DSM 44152 | GCA_001722445.1 | 4.72 | 65.8 |
| Mycobacterium diernhoferi | ATCC | GCA_019456655.1 | 6.00 | 67.9 |
| | 19340 | | | |
| Mycobacterium paraffinicum | M11 | GCA_001907675.1 | 6.48 | 67.6 |
| Mycobacterium angelicum | DSM 45057 | GCA_002086155.1 | 6.66 | 66.3 |
| Mycobacterium bouchedurhonense | DSM 45439 | GCA_002086165.1 | 5.90 | 68.6 |
| Mycobacterium heidelbergense | JCM 14842 | GCA_010730745.1 | 5.05 | 67.9 |
| Mycobacterium mantenii | JCM 18113 | GCA_010731775.1 | 6.19 | 66.9 |
| Mycobacterium marseillense | JCM 17324 | GCA_010731675.1 | 5.51 | 67.7 |
| Mycobacterium noviomagense | JCM 16367 | GCA_010731635.1 | 4.78 | 65.8 |
| Mycobacterium paraseoulense | JCM 16952 | GCA_010731655.1 | 6.09 | 67.9 |
| Mycobacterium branderi | JCM 12687 | GCA_010728725.1 | 5.98 | 66.5 |
| Mycobacterium alsense | DSM 45230 | GCA_002086635.1 | 5.69 | 69.3 |
| Mycobacterium shinjukuense | JCM 14233 | GCA_010730055.1 | 4.50 | 67.8 |
| Mycobacterium timonense | JCM 30726 | GCA_010723675.1 | 5.70 | 67.9 |
| Mycobacterium florentinum | JCM 14740 | GCA_010730355.1 | 6.22 | 66.4 |
| Mycobacterium paraense | IEC 26 | GCA_002101815.1 | 5.62 | 69.3 |
| Mycobacterium kubicae | JCM 13573 | GCA_015689175.1 | 6.02 | 66.0 |
| Mycobacterium saskatchewanense | JCM 13016 | GCA_010729105.1 | 6.01 | 68.3 |
| Mycobacterium palustre | DSM 44572 | GCA_002101785.1 | 6.04 | 68.5 |
| Mycobacterium conspicuum | JCM 14738 | GCA_010730195.1 | 6.24 | 67.4 |
| Mycobacterium fragae | DSM 45731 | GCA_002102185.1 | 4.73 | 66.0 |
| Mycobacterium parmense | JCM 14742 | GCA_010730575.1 | 5.95 | 68.4 |
| Mycobacterium lacus | JCM 15657 | GCA_010731535.1 | 5.09 | 66.9 |
| Mycobacterium koreense | JCM 19956 | GCA_010731835.1 | 4.16 | 69.3 |
| Mycobacterium persicum | DSM | GCA_002086675.1 | 6.17 | 66.2 |
| | 104278 | | | |
| Mycobacterium talmoniae | ATCC | GCA_002967005.1 | 5.13 | 68.2 |
| | BAA-2683 | | | |
| Mycobacterium aquaticum | RW6 | GCA_002086485.1 | 7.93 | 66.5 |
| Mycobacterium lehmannii | CECT 8763 | GCA_002245535.1 | 5.50 | 66.7 |
| Mycobacterium neumannii | CECT 8766 | GCA_002245615.1 | 5.38 | 66.8 |
| Mycobacterium virginiense | DSM | GCA_022374935.1 | 4.98 | 67.4 |
| | 100883 | | | |
| Mycobacterium ahvazicum | AFP003 | GCA_900176255.2 | 6.12 | 66.2 |
| Mycobacterium uberis | Jura | GCA_003408705.1 | 3.12 | 57.5 |
| Mycobacterium decipiens | TBL | GCA_002104675.1 | 5.23 | 65.5 |
| | 1200985 | | | |
| Mycobacterium syngnathidarum | 27335 | GCA_001942625.1 | 6.34 | 66.2 |
| Mycobacterium kyogaense | NCTC | GCA_003254575.1 | 5.76 | 67.9 |
| | 11659 | | | |
| Mycobacterium basiliense | 901379 | GCA_900292015.1 | 5.61 | 65.0 |
| Mycobacterium pseudokansasii | MK142 | GCA_900566075.1 | 6.43 | 66.2 |
| Mycobacterium innocens | MK13 | GCA_900566055.1 | 6.19 | 66.1 |
| Mycobacterium attenuatum | MK41 | GCA_900566085.1 | 6.53 | 65.8 |
| Mycobacterium tilburgii | MEPHI | GCA_902168065.1 | 3.24 | 66.3 |
| Mycobacterium botniense | JCM 17322 | GCA_010723305.1 | 4.34 | 65.7 |
| Mycobacterium bourgelatii | JCM 30725 | GCA_010723575.1 | 6.91 | 65.7 |
| Mycobacterium novum | JCM 6391 | GCA_010726505.1 | 4.46 | 68.6 |
| Mycobacterium cookii | JCM 12404 | GCA_010727945.1 | 5.32 | 66.0 |
| Mycobacterium shottsii | JCM 12657 | GCA_010728525.1 | 5.97 | 65.5 |
| Mycobacterium seoulense | JCM 16018 | GCA_010731595.1 | 5.53 | 68.3 |
| Mycobacterium stromatepiae | JCM 17783 | GCA_010731715.1 | 6.21 | 66.0 |
| Mycobacterium neglectum | CECT 8778 | GCA_002591975.1 | 6.21 | 65.3 |
| Mycobacterium palauense | CECT 8779 | GCA_002592005.1 | 6.23 | 69.4 |
| Mycobacterium terramassiliense | AB308 | GCA_900157385.1 | 6.03 | 68.4 |
| Mycobacterium rhizamassiliense | AB57 | GCA_900157375.1 | 6.02 | 67.2 |
| Mycobacterium | AB215 | GCA_900157365.1 | 6.25 | 65.9 |
| numidiamassiliense | | | | |
| Mycobacterium simulans | FI-09026 | GCA_900232995.1 | 6.19 | 64.6 |
| Mycobacterium helveticum | 16-83 | GCA_007714205.1 | 5.77 | 68.7 |
| Mycobacterium ostraviense | 241/15 | GCA_002705925.1 | 6.10 | 66.2 |
| Mycobacterium spongiae | FSD4b-SM | GCA_018278905.1 | 5.58 | 65.6 |
| Mycobacterium vicinigordonae | 24 | GCA_013466425.1 | 6.27 | 65.4 |
| Mycobacterium paraterrae | DSM 45127 | GCA_022430545.1 | 5.52 | 65.5 |
| Mycobacterium senriense | TY59 | GCA_019668465.1 | 5.83 | 67.1 |
| Mycobacterium holsaticum | JCM 12374 | GCA_019645835.1 | 5.62 | 67.2 |
| Mycobacterium tuberculosis subsp. | ATCC | GCA_002982225.1 | 4.30 | 65.6 |
| caprae | BAA-824 | | | |
| Mycobacterium celatum | DSM 44243 | GCA_002101595.1 | 4.72 | 67.0 |
| Mycobacterium triplex | DSM 44626 | GCA_000689255.1 | 6.38 | 66.6 |
| Mycobacterium kyorinense | DSM 45166 | GCA_002101735.1 | 5.61 | 66.8 |
| Mycobacterium lepromatosis | FJ924 | GCA_000975265.2 | 3.27 | 57.9 |
| Mycobacterium nebraskense | DSM 44803 | GCA_002102255.1 | 6.73 | 66.6 |
| Mycobacterium senegalense | ATCC | GCA_019645875.1 | 6.09 | 66.6 |
| | 35796 | | | |
| Mycobacterium heraklionense | Davo | GCA_001021505.1 | 5.11 | 67.9 |
| Mycobacterium heckeshornense | JCM 15655 | GCA_016592155.1 | 4.96 | 65.9 |
| Mycobacterium goodii | ATCC | GCA_022370755.1 | 6.74 | 67.1 |
| | 700504 | | | |
| Mycobacterium intracellulare | DSM 44623 | GCA_002219285.1 | 6.13 | 67.7 |
| subsp. chimaera | | | | |
| Mycobacterium gordonae | DSM 44160 | GCA_002101675.1 | 7.60 | 66.6 |
| Mycobacterium malmoense | ATCC | GCA_019645855.1 | 5.32 | 67.0 |
| | 29571 | | | |
| Mycobacterium paraintracellulare | JCM 30622 | GCA_010731935.1 | 5.53 | 68.1 |
| Mycobacterium riyadhense | DSM 45176 | GCA_002101845.1 | 6.27 | 65.3 |
| Mycobacterium dioxanotrophicus | PH-06 | GCA_002157835.1 | 8.08 | 66.4 |
| Mycobacterium grossiae | DSM | GCA_008329645.1 | 5.68 | 70.5 |
| | 104744 | | | |
| Mycobacterium tuberculosis subsp. | ATCC | GCA_002982275.1 | 4.33 | 65.6 |
| pinnipedii | BAA-688 | | | |
| Mycobacterium montefiorense | BS | GCA_003112775.1 | 5.74 | 65.1 |
| Mycobacterium paragordonae | 49061 | GCA_003614435.1 | 7.22 | 66.9 |
| Mycobacterium frederiksbergense | LB 501T | GCA_012223425.1 | 6.71 | 67.1 |
| Mycobacterium shigaense | JCM 32072 | GCA_002356315.1 | 5.23 | 67.3 |
| Mycobacterium gastri | DSM 43505 | GCA_002102175.1 | 5.82 | 66.2 |
| Mycobacterium genavense | ATCC | GCA_000526915.1 | 4.94 | 66.9 |
| | 51324 | | | |
| Mycobacterium hodleri | S5.20 | GCA_006439015.1 | 6.38 | 65.9 |
| Mycobacterium pyrenivorans | JCM 15927 | GCA_001314105.1 | 5.13 | 66.3 |
| Mycobacterium crocinum | JCM 16369 | GCA_022370635.1 | 6.17 | 66.6 |
| Mycobacterium pseudoshottsii | JCM 15466 | GCA_003584745.1 | 6.06 | 65.6 |
| Mycobacterium haemophilu | ATCC | GCA_000340435.3 | 4.24 | 63.9 |
| | 29548 | | | |
| Mycobacterium simiae | JCM 12377 | GCA_010727605.1 | 5.79 | 66.2 |
| Mycobacterium bohemicum | DSM 44277 | GCA_001053185.1 | 5.10 | 68.8 |
| Mycobacterium asiaticum | DSM 44297 | GCA_000613245.1 | 5.94 | 66.2 |
| Mycobacterium arosiense | DSM 45069 | GCA_002086125.1 | 5.98 | 66.8 |
| Mycobacterium intracellulare | KCTC | GCA_000418535.2 | 5.66 | 78.0 |
| subsp. yongonense | 19555 | | | |
| Mycobacterium orygis | 51145 | GCA_015265495.2 | 4.35 | 65.6 |
| Mycobacterium rufum | JCM 16372 | GCA_022374875.1 | 6.01 | 69.4 |
| Mycobacterium pallens | JCM 16370 | GCA_019456675.1 | 6.09 | 66.4 |
| Mycobacterium parascrofulaceum | ATCC | GCA_000164135.1 | 6.56 | 67.6 |
| | BAA-614 | | | |
| Mycobacterium tuberculosis subsp. | ATCC | GCA_904810335.1 | 4.37 | 65.6 |
| microti | 34782 | | | |
| Mycobacterium intracellulare | MTCC 9506 | GCA_021183785.1 | 5.59 | 68.1 |
| subsp. intracellulare | | | | |
| Mycobacterium liflandii | ASM001 | GCA_022354805.1 | 6.17 | 65.6 |
| Mycobacterium colombiense | CECT 3035 | GCA_002105755.1 | 5.58 | 68.1 |
| Mycobacterium xenopi | JCM 15661 | GCA_009936235.1 | 4.92 | 65.9 |
| Mycobacterium kansasii | ATCC | GCA_000157895.1 | 6.58 | 66.2 |
| | 12478 | | | |
| Mycobacterium ulcerans | ATCC | GCA_022374915.1 | 5.62 | 65.5 |
| | 19423 | | | |
| Mycobacterium leprae | TN | GCA_000195855.1 | 3.27 | 57.8 |
| Mycobacterium marinum | CCUG | GCA_003391395.1 | 6.45 | 65.7 |
| | 20998 | | | |
| Mycobacterium intracellulare | ATCC | GCA_000277125.1 | 5.40 | 68.1 |
| | 13950 | | | |
| Mycobacterium canettii | CIPT | GCA_000328785.1 | 4.53 | 65.5 |
| | 140070010 | | | |
| Mycobacterium tuberculosis subsp. | ATCC | GCA_002982285.1 | 4.31 | 65.5 |
| bovis | 19210 | | | |
| Mycobacterium tuberculosis subsp. | ATCC | GCA_000666065.1 | 4.35 | 65.5 |
| africanum | 25420 | | | |
| Mycobacterium avium subsp. | DSM 44156 | GCA_009741445.1 | 4.96 | 69.3 |
| avium | | | | |
| Mycobacterium avium subsp. | DSM 44135 | GCA_013357385.1 | 4.84 | 69.3 |
| paratuberculosis | | | | |
| Mycobacterium avium subsp. | H87 | GCA_001936215.1 | 5.63 | 68.8 |
| hominissuis | | | | |
| Mycobacterium avium subsp. | ATCC | GCA_000504975.1 | 4.71 | 69.2 |
| silvaticum | 49884 | | | |
| Mycobacterium tuberculosis | H37Rv | GCA_000277735.2 | 4.41 | 65.6 |
| | | Average = | 5.64 | 66.7 |
| | | St. Deviation = | 0.89 | 2.13 |

TABLE 5
Mycobacteroides genomes analyzed in this study

| | | | | GC |
|---|---|---|---|---|
| Species | Strain | Assembly | Genome Size (Mbp) | % |
| Mycobacteroides abscessus | ATCC | GCF_000069185.1 | 5.09 | 64.1 |
| subsp. abscessus | 19977 | | | |
| Mycobacteroides bolletii subsp. | BD | GCA_003609715.1 | 5.08 | 64.1 |
| bolletii | | | | |
| Mycobacteroides massiliense | JCM 15300 | GCA_000497265.2 | 4.98 | 64.1 |
| subsp. massiliense | | | | |
| Mycobacteroides chelonae | CCUG | GCA_001632805.1 | 5.03 | 63.9 |
| | 47445 | | | |
| Mycobacteroides | DSM 43276 | GCA_004924335.1 | 4.78 | 64.3 |
| salmoniphilum | | | | |
| Mycobacteroides franklinii | DSM 45524 | GCA_004355025.1 | 5.41 | 65.1 |
| Mycobacteroides saopaulense | EPM10906 | GCA_001456355.1 | 4.65 | 64.8 |
| Mycobacteroides | CCUG | GCA_001605725.1 | 5.57 | 64.3 |
| immunogenum | 47286 | | | |
| | | Average | 5.07 | 64.34 |
| | | St. Deviation | 0.30 | 0.41 |

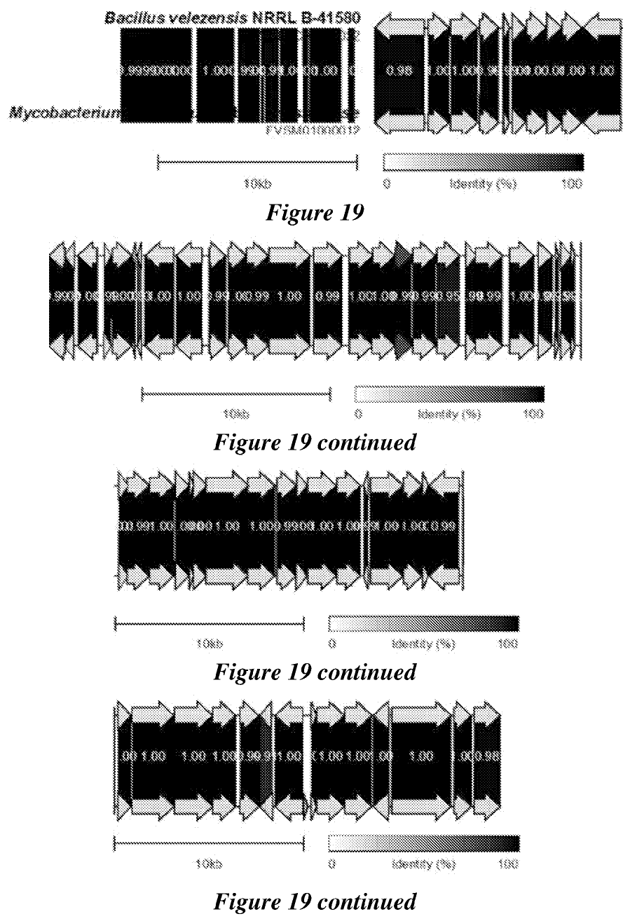

To identify the most likely origin of the phosphonate/phosphinate biosynthetic gene cluster in the AA3 assembly, the contig containing pepM was analyzed. The gene was part of a 155 kbp contig (accession no. FVSM01000012) with a mean GC content of 46.7%. This is within the expected average GC-content of B. velezensis (45.2-47.0%; Table 6) but significantly lower than the values for Mycobacterium (66.7%) and Mycobacteroides (64.3%) (Table 4 and Table 5). Indeed, the sequence surrounding pepM was nearly completely syntenic to the corresponding 100 kb region from Bacillus velezensis NRRL B-41580 (FIG. 19). Furthermore, the predicted proteins within the immediate pepM neighborhood of AA3 shared ≥97% sequence identity with their corresponding homologs in B. velezensis NRRL B-41580 (FIG. 2; Table 7). Altogether, these results indicate Bacillus as the actual source of the phosphonate/phosphinate biosynthetic gene cluster rather than horizontal gene transfer into a Mycobacterium or Mycobacteriodetes. In fact, reciprocal analysis of publicly available Mycobacterium and Mycobacteriodetes genomes indicated the complete absence of phosphonate/phosphinate biosynthetic gene clusters from these genera except for those with contaminated datasets (e.g. AA3).

TABLE 6
Bacillus velezensis genomes analyzed in this study

| Species | Strain | Assembly Accession | Genome Size(Mbp) | GC % |
|---|---|---|---|---|
| Bacillus velezensis | 83 | GCA_004101805.1 | 4.00 | 46.4 |
| Bacillus velezensis | 157 | GCA_002216755.1 | 4.02 | 46.4 |
| Bacillus velezensis | 504 | GCA_022385235.1 | 3.91 | 46.6 |
| Bacillus velezensis | 906 | GCA_024408125.1 | 3.95 | 46.6 |
| Bacillus velezensis | 10075 | GCA_002893845.1 | 4.34 | 46.1 |
| Bacillus velezensis | 2-Aug | GCA_003047025.1 | 3.89 | 46.7 |
| Bacillus velezensis | 131-4 | GCA_003047045.1 | 3.89 | 46.7 |
| Bacillus velezensis | 19573-3 | GCA_019551675.1 | 3.99 | 46.6 |
| Bacillus velezensis | 1B-23 | GCA_003854915.1 | 4.14 | 46.1 |
| Bacillus velezensis | 2e | GCA_023516175.1 | 3.97 | 46.5 |
| Bacillus velezensis | 9912D | GCA_001857985.1 | 4.24 | 46.0 |
| Bacillus velezensis | 9D-6 | GCA_002105595.1 | 3.96 | 46.4 |
| Bacillus velezensis | A2 | GCA_013201135.1 | 3.93 | 46.5 |
| Bacillus velezensis | A4P130 | GCA_021228895.1 | 4.24 | 45.9 |
| Bacillus velezensis | AB191 | GCA_021390055.1 | 3.93 | 46.5 |
| Bacillus velezensis | AD-3 | GCA_017347625.1 | 4.27 | 45.9 |
| Bacillus velezensis | AD8 | GCA_017742975.1 | 3.89 | 46.6 |
| Bacillus velezensis | Ag75 | GCA_023921085.1 | 3.98 | 46.5 |
| Bacillus velezensis | AGVL-005 | GCA_002795885.1 | 4.15 | 46.0 |
| Bacillus velezensis | AK-0 | GCA_014706595.1 | 3.97 | 46.5 |
| Bacillus velezensis | AL7 | GCA_009663035.1 | 3.89 | 46.6 |
| Bacillus velezensis | ANSB01E | GCA_004329055.1 | 3.93 | 46.5 |
| Bacillus velezensis | AP3 | GCA_022760025.1 | 4.02 | 46.5 |
| Bacillus velezensis | AS43.3 | GCA_000319475.1 | 3.96 | 46.6 |
| Bacillus velezensis | At1 | GCA_006489275.1 | 3.89 | 46.7 |
| Bacillus velezensis | ATR2 | GCA_002761535.1 | 4.01 | 46.3 |
| Bacillus velezensis | B-001 | GCA_020995365.1 | 4.07 | 46.2 |
| Bacillus velezensis | B-4 | GCA_003390415.1 | 3.92 | 46.7 |
| Bacillus velezensis | B1 | GCA_023614465.1 | 3.99 | 46.5 |
| Bacillus velezensis | B25 | GCA_001536925.1 | 3.86 | 46.7 |
| Bacillus velezensis | B268 | GCA_013201465.1 | 3.91 | 46.6 |
| Bacillus velezensis | B4-7 | GCA_019603335.1 | 3.93 | 46.5 |
| Bacillus velezensis | Ba-0321 | GCA_024508215.1 | 4.10 | 46.3 |
| Bacillus velezensis | BA-26 | GCA_009789615.1 | 4.04 | 46.4 |
| Bacillus velezensis | Bac57 | GCA_003667745.1 | 4.23 | 45.9 |
| Bacillus velezensis | BCSo1 | GCA_003855275.1 | 3.71 | 47.0 |
| Bacillus velezensis | BIM B-1312D | GCA_011769925.1 | 3.93 | 46.5 |
| Bacillus velezensis | BIM B-439D | GCA_003546955.1 | 3.98 | 46.5 |
| Bacillus velezensis | BIM B-454D | GCA_019880485.1 | 4.22 | 45.9 |
| Bacillus velezensis | BIOMA BV10 | GCA_013694265.1 | 3.92 | 46.6 |
| Bacillus velezensis | BP1.2A | GCA_013285085.2 | 3.92 | 46.5 |
| Bacillus velezensis | BR-01 | GCA_021397795.1 | 4.03 | 46.4 |
| Bacillus velezensis | BS-37 | GCA_003076535.1 | 4.01 | 46.5 |
| Bacillus velezensis | BS-G1 | GCA_019222745.1 | 3.93 | 46.5 |
| Bacillus velezensis | BSC16a | GCA_014841075.1 | 3.93 | 46.5 |
| Bacillus velezensis | BT2.4 | GCA_013284785.2 | 3.92 | 46.4 |
| Bacillus velezensis | BvL03 | GCA_009664145.1 | 3.98 | 46.6 |
| Bacillus velezensis | BY6 | GCA_012275165.1 | 3.91 | 46.6 |
| Bacillus velezensis | C1 | GCA_022700875.1 | 4.08 | 46.3 |
| Bacillus velezensis | C5 | GCA_024499135.1 | 3.93 | 46.5 |
| Bacillus velezensis | CACC 316 | GCA_009931675.1 | 4.05 | 46.3 |
| Bacillus velezensis | CAU B946 | GCA_000283695.1 | 4.02 | 46.5 |
| Bacillus velezensis | CBMB205 | GCA_002117165.1 | 3.93 | 46.5 |
| Bacillus velezensis | CC09 | GCA_001593395.2 | 4.17 | 46.1 |
| Bacillus velezensis | CF57 | GCA_021559715.1 | 4.00 | 46.4 |
| Bacillus velezensis | CGMCC 11640 | GCA_002968415.1 | 4.39 | 45.6 |
| Bacillus velezensis | CK17 | GCA_023823715.1 | 3.96 | 46.6 |
| Bacillus velezensis | CL-4 | GCA_019720775.1 | 4.06 | 46.3 |
| Bacillus velezensis | CLA178 | GCA_014495825.1 | 4.07 | 46.2 |
| Bacillus velezensis | CMF18 | GCA_023823735.1 | 3.96 | 46.5 |
| Bacillus velezensis | CMT-6 | GCA_002845365.1 | 3.93 | 46.6 |
| Bacillus velezensis | CN026 | GCA_002796465.1 | 4.00 | 46.4 |
| Bacillus velezensis | CSUSB3 | GCA_020162235.1 | 4.09 | 46.3 |
| Bacillus velezensis | DH8043 | GCA_009856975.1 | 3.97 | 46.2 |
| Bacillus velezensis | DKU_NT_04 | GCA_002944585.1 | 4.33 | 45.2 |
| Bacillus velezensis | DMB05 | GCA_020149525.1 | 4.14 | 46.2 |
| Bacillus velezensis | DMB06 | GCA_020149955.1 | 4.16 | 46.2 |
| Bacillus velezensis | DMB07 | GCA_020150015.1 | 4.18 | 45.6 |
| Bacillus velezensis | DR-08 | GCA_003045165.1 | 3.93 | 46.5 |
| Bacillus velezensis | DSYZ | GCA_003285085.1 | 4.32 | 45.7 |
| Bacillus velezensis | DTU001 | GCA_015291865.1 | 3.93 | 46.6 |
| Bacillus velezensis | EN01 | GCA_013122275.1 | 4.03 | 46.5 |
| Bacillus velezensis | ES2-4 | GCA_023516195.1 | 3.93 | 46.5 |
| Bacillus velezensis | FIO1408 | GCA_015732475.1 | 4.18 | 46.2 |
| Bacillus velezensis | FJAT-45028 | GCA_009834085.1 | 3.99 | 46.5 |
| Bacillus velezensis | FJAT-46737 | GCA_008727675.1 | 4.00 | 46.5 |
| Bacillus velezensis | FJAT-52631 | GCA_009362235.1 | 3.93 | 46.5 |
| Bacillus velezensis | FZB42 | GCA_000015785.2 | 3.92 | 46.5 |
| Bacillus velezensis | G341 | GCA_001023595.1 | 4.01 | 46.5 |
| Bacillus velezensis | GB03 | GCA_000508125.2 | 3.89 | 46.6 |
| Bacillus velezensis | GFP-2 | GCA_002850475.1 | 3.98 | 46.4 |
| Bacillus velezensis | GH1-13 | GCA_002005345.1 | 4.14 | 45.9 |
| Bacillus velezensis | GMEKP1 | GCA_018866365.1 | 4.09 | 46.4 |
| Bacillus velezensis | GQJK49 | GCA_002192235.1 | 3.93 | 46.5 |
| Bacillus velezensis | GS-1 | GCA_019449395.1 | 4.03 | 46.3 |
| Bacillus velezensis | GSBZ09 | GCA_021184205.1 | 4.12 | 46.2 |
| Bacillus velezensis | GUAL210 | GCA_016834435.1 | 4.01 | 46.4 |
| Bacillus velezensis | GUIA | GCA_022869125.1 | 3.93 | 46.5 |
| Bacillus velezensis | GUMT319 | GCA_016766595.1 | 3.94 | 46.6 |
| Bacillus velezensis | GY65 | GCA_014961825.1 | 3.93 | 46.5 |
| Bacillus velezensis | GYL4 | GCA_003071465.1 | 3.98 | 46.5 |
| Bacillus velezensis | H208 | GCA_023516155.1 | 3.93 | 46.5 |
| Bacillus velezensis | HAB-2 | GCA_014211995.1 | 3.89 | 46.6 |
| Bacillus velezensis | HMB26553 | GCA_023546985.1 | 4.34 | 46.1 |
| Bacillus velezensis | HN-Q-8 | GCA_009738165.1 | 4.00 | 46.4 |
| Bacillus velezensis | HNU24 | GCA_022370935.1 | 3.93 | 46.5 |
| Bacillus velezensis | Htq6 | GCA_012226065.1 | 3.89 | 46.5 |
| Bacillus velezensis | Hx05 | GCA_003150855.2 | 3.91 | 46.5 |
| Bacillus velezensis | IM1 | GCA_022494925.1 | 3.92 | 46.6 |
| Bacillus velezensis | J17-4 | GCA_018775385.1 | 3.93 | 46.5 |
| Bacillus velezensis | J7-1 | GCA_003047005.1 | 3.89 | 46.7 |
| Bacillus velezensis | JIN4 | GCA_020911825.1 | 3.90 | 46.6 |
| Bacillus velezensis | JJ-D34 | GCA_000987825.1 | 4.11 | 46.2 |
| Bacillus velezensis | JJ47 | GCA_021650855.1 | 3.93 | 46.5 |
| Bacillus velezensis | JS25R | GCA_000769555.1 | 4.01 | 46.4 |
| Bacillus velezensis | JSRB 08 | GCA_014048385.1 | 4.14 | 45.9 |
| Bacillus velezensis | JSRB 166 | GCA_014048285.1 | 4.07 | 46.4 |
| Bacillus velezensis | JT3-1 | GCA_003612755.1 | 3.93 | 46.5 |
| Bacillus velezensis | JTYP2 | GCA_002072695.1 | 3.93 | 46.5 |
| Bacillus velezensis | K01 | GCA_013701805.1 | 3.93 | 46.5 |
| Bacillus velezensis | K203 | GCA_022344025.1 | 4.03 | 46.4 |
| Bacillus velezensis | K26 | GCA_003432425.1 | 4.05 | 46.1 |
| Bacillus velezensis | KD1 | GCA_001752685.2 | 3.92 | 46.7 |
| Bacillus velezensis | KKLW | GCA_013348705.1 | 3.92 | 46.5 |
| Bacillus velezensis | KMU01 | GCA_015277495.1 | 3.93 | 46.5 |
| Bacillus velezensis | KOF112 | GCA_018406485.1 | 3.93 | 46.5 |
| Bacillus velezensis | KS04AU | GCA_022488425.1 | 4.01 | 46.5 |
| Bacillus velezensis | L-1 | GCA_002688525.1 | 4.09 | 46.5 |
| Bacillus velezensis | L-H15 | GCA_000833005.1 | 3.91 | 46.7 |
| Bacillus velezensis | L-S60 | GCA_000973485.1 | 3.90 | 46.7 |
| Bacillus velezensis | LABIM22 | GCA_013401395.2 | 4.00 | 46.4 |
| Bacillus velezensis | LABIM40 | GCA_002556565.1 | 3.97 | 46.5 |
| Bacillus velezensis | LABIM44 | GCA_019334525.1 | 4.05 | 46.3 |
| Bacillus velezensis | LB002 | GCA_004337655.1 | 4.08 | 46.5 |
| Bacillus velezensis | LBUM1082 | GCA_016065635.1 | 3.93 | 46.6 |
| Bacillus velezensis | LBUM279 | GCA_016415725.1 | 4.17 | 46.0 |
| Bacillus velezensis | LBY-1 | GCA_019285615.1 | 4.28 | 46.1 |
| Bacillus velezensis | LC1 | GCA_008802875.1 | 3.93 | 46.5 |
| Bacillus velezensis | LDO2 | GCA_003073455.1 | 3.95 | 46.5 |
| Bacillus velezensis | LF01 | GCA_015239615.1 | 3.97 | 46.6 |
| Bacillus velezensis | LG37 | GCA_006974185.1 | 3.93 | 46.5 |
| Bacillus velezensis | LOH112 | GCA_022313395.1 | 4.36 | 45.8 |
| Bacillus velezensis | LPL-K103 | GCA_000815275.2 | 3.93 | 46.6 |
| Bacillus velezensis | LPL061 | GCA_011032725.1 | 3.91 | 46.7 |
| Bacillus velezensis | LS69 | GCA_001687745.1 | 3.92 | 46.5 |
| Bacillus velezensis | Lzh-a42 | GCA_002844125.1 | 4.25 | 46.0 |
| Bacillus velezensis | LZN01 | GCA_019754015.1 | 3.97 | 46.5 |
| Bacillus velezensis | M75 | GCA_001723585.1 | 4.01 | 46.6 |
| Bacillus velezensis | MH25 | GCA_003860445.1 | 4.12 | 46.5 |
| Bacillus velezensis | Mr12 | GCA_016406185.1 | 4.00 | 46.5 |
| Bacillus velezensis | MV2 | GCA_013875015.1 | 4.19 | 45.6 |
| Bacillus velezensis | N3 | GCA_023373485.1 | 4.16 | 46.1 |
| Bacillus velezensis | NAU-B3 | GCA_000493375.1 | 4.20 | 46.0 |
| Bacillus velezensis | NJ13 | GCA_023822685.1 | 3.93 | 46.5 |
| Bacillus velezensis | NJAU-Z9 | GCA_002238395.1 | 3.87 | 46.8 |
| Bacillus velezensis | NJN-6 | GCA_000973585.1 | 4.05 | 46.6 |
| Bacillus velezensis | NKG-1 | GCA_002741705.1 | 4.20 | 46.3 |
| Bacillus velezensis | NST6 | GCA_014180705.2 | 4.14 | 46.0 |
| Bacillus velezensis | NY12-2 | GCA_003721475.1 | 4.07 | 46.3 |
| Bacillus velezensis | NZ4 | GCA_018732185.1 | 4.09 | 46.3 |
| Bacillus velezensis | ONU 553 | GCA_008244925.1 | 3.93 | 46.7 |
| Bacillus velezensis | PEBA20 | GCA_016859395.1 | 4.28 | 45.9 |
| Bacillus velezensis | PHP1601 | GCA_024218535.1 | 3.93 | 46.5 |
| Bacillus velezensis | Pm9 | GCA_014117505.1 | 3.89 | 46.7 |
| Bacillus velezensis | Q12 | GCA_018771665.1 | 4.18 | 46.1 |
| Bacillus velezensis | QST713 | GCA_003073255.1 | 4.23 | 45.9 |
| Bacillus velezensis | R 4.6 | GCA_023586845.1 | 4.18 | 46.0 |
| Bacillus velezensis | R-71003 | GCA_022385175.1 | 4.21 | 46.1 |
| Bacillus velezensis | S141 | GCA_003573875.1 | 3.97 | 46.5 |
| Bacillus velezensis | S3-1 | GCA_001685645.1 | 3.93 | 46.5 |
| Bacillus velezensis | S4 | GCA_011784665.1 | 4.07 | 46.4 |
| Bacillus velezensis | Sam8H1 | GCA_016904925.1 | 4.13 | 46.1 |
| Bacillus velezensis | SC60 | GCA_023278305.1 | 3.96 | 46.5 |
| Bacillus velezensis | SCDB 291 | GCA_002243325.2 | 4.16 | 46.4 |
| Bacillus velezensis | SCGB 1 | GCA_002310315.1 | 4.09 | 46.4 |
| Bacillus velezensis | SCGB 574 | GCA_002327165.1 | 3.99 | 46.5 |
| Bacillus velezensis | SF327 | GCA_022371095.1 | 4.08 | 46.5 |
| Bacillus velezensis | SQR9 | GCA_000685725.1 | 4.12 | 46.1 |
| Bacillus velezensis | SRCM100072 | GCA_002201935.1 | 4.00 | 46.3 |
| Bacillus velezensis | SRCM101368 | GCA_009914175.1 | 4.09 | 46.3 |
| Bacillus velezensis | SRCM101413 | GCA_002201975.1 | 4.21 | 46.2 |
| Bacillus velezensis | SRCM102741 | GCA_009913315.1 | 4.02 | 46.5 |
| Bacillus velezensis | SRCM102742 | GCA_009913335.1 | 4.02 | 46.5 |
| Bacillus velezensis | SRCM102743 | GCA_009913355.1 | 3.96 | 46.6 |
| Bacillus velezensis | SRCM102744 | GCA_009913375.1 | 4.10 | 46.2 |
| Bacillus velezensis | SRCM102746 | GCA_009913475.1 | 3.96 | 46.6 |
| Bacillus velezensis | SRCM102747 | GCA_009913495.1 | 4.10 | 46.2 |
| Bacillus velezensis | SRCM102752 | GCA_009914135.1 | 3.97 | 46.4 |
| Bacillus velezensis | SRCM102755 | GCA_009913295.1 | 4.04 | 46.3 |
| Bacillus velezensis | SRCM103616 | GCA_004119855.1 | 4.24 | 46.1 |
| Bacillus velezensis | SRCM103691 | GCA_004119575.1 | 4.14 | 46.2 |
| Bacillus velezensis | SRCM103788 | GCA_004119735.1 | 4.14 | 46.2 |
| Bacillus velezensis | SW5 | GCA_022870725.1 | 4.13 | 46.1 |
| Bacillus velezensis | SWUJ1 | GCA_020084885.1 | 3.93 | 46.5 |
| Bacillus velezensis | sx01604 | GCA_002057535.1 | 3.93 | 46.5 |
| Bacillus velezensis | SYBC H47 | GCA_001854345.1 | 3.88 | 46.4 |
| Bacillus velezensis | SYP-B637 | GCA_008367895.1 | 3.92 | 46.5 |
| Bacillus velezensis | T20E-257 | GCA_002205715.1 | 3.90 | 46.7 |
| Bacillus velezensis | TB1501 | GCA_002237515.1 | 3.98 | 46.5 |
| Bacillus velezensis | TB918 | GCA_016888865.1 | 3.98 | 46.5 |
| Bacillus velezensis | TJ02 | GCA_002764075.1 | 4.06 | 46.4 |
| Bacillus velezensis | TPS3N | GCA_024138575.1 | 3.93 | 46.6 |
| Bacillus velezensis | TrigoCor1448 | GCA_000583065.1 | 3.96 | 46.5 |
| Bacillus velezensis | UA0182 | GCA_023573345.1 | 3.89 | 46.4 |
| Bacillus velezensis | UA0188 | GCA_023573285.1 | 3.87 | 46.4 |
| Bacillus velezensis | UA0195 | GCA_023573265.1 | 3.89 | 46.4 |
| Bacillus velezensis | UA0229 | GCA_023573325.1 | 3.89 | 46.4 |
| Bacillus velezensis | UA0244 | GCA_023573505.1 | 3.89 | 46.4 |
| Bacillus velezensis | UA0276 | GCA_023573205.1 | 3.87 | 46.4 |
| Bacillus velezensis | UA0311 | GCA_023573245.1 | 3.87 | 46.4 |
| Bacillus velezensis | UA0323 | GCA_023573225.1 | 3.87 | 46.4 |
| Bacillus velezensis | UA1365 | GCA_023573165.1 | 3.87 | 46.4 |
| Bacillus velezensis | UB2017 | GCA_011100445.1 | 3.93 | 46.5 |
| Bacillus velezensis | UCMB-5033 | GCA_000455565.1 | 4.07 | 46.2 |
| Bacillus velezensis | UCMB5007 | GCA_006489255.1 | 3.98 | 46.6 |
| Bacillus velezensis | UCMB5036 | GCA_000341875.1 | 3.91 | 46.6 |
| Bacillus velezensis | UCMB5044 | GCA_006489235.1 | 3.98 | 46.6 |
| Bacillus velezensis | UCMB5113 | GCA_000455585.1 | 3.89 | 46.7 |
| Bacillus velezensis | UCMB5140 | GCA_012647845.1 | 3.98 | 46.5 |
| Bacillus velezensis | UFLA258 | GCA_004799565.1 | 3.95 | 46.6 |
| Bacillus velezensis | US1 | GCA_018771705.1 | 4.13 | 46.0 |
| Bacillus velezensis | V 3.14 | GCA_023586825.1 | 4.18 | 46.0 |
| Bacillus velezensis | VCN56 | GCA_018629015.1 | 3.97 | 46.5 |
| Bacillus velezensis | VTX20 | GCA_018415955.1 | 3.89 | 46.7 |
| Bacillus velezensis | VTX9 | GCA_018398955.1 | 4.05 | 46.4 |
| Bacillus velezensis | VY03 | GCA_017948225.1 | 3.94 | 46.7 |
| Bacillus velezensis | W1 | GCA_003265265.1 | 4.24 | 45.8 |
| Bacillus velezensis | WB | GCA_019464595.1 | 3.90 | 46.7 |
| Bacillus velezensis | WLYS23 | GCA_013367755.1 | 3.93 | 46.5 |
| Bacillus velezensis | WRN014 | GCA_006965525.1 | 4.06 | 46.3 |
| Bacillus velezensis | WSM-1 | GCA_016801735.1 | 3.93 | 46.5 |
| Bacillus velezensis | Y4_39 | GCA_014764325.1 | 3.89 | 46.7 |
| Bacillus velezensis | Y816 | GCA_018223705.1 | 3.91 | 46.6 |
| Bacillus velezensis | Yao | GCA_021495995.1 | 3.95 | 46.6 |
| Bacillus velezensis | YAU B9601-Y2 | GCA_000262385.1 | 4.24 | 45.9 |
| Bacillus velezensis | YB-130 | GCA_014358035.1 | 3.98 | 46.5 |
| Bacillus velezensis | YJ11-1-4 | GCA_000988345.1 | 4.01 | 46.4 |
| Bacillus velezensis | YYC | GCA_019163475.1 | 3.97 | 46.5 |
| Bacillus velezensis | ZF145 | GCA_014622705.1 | 3.93 | 46.5 |
| Bacillus velezensis | ZF2 | GCA_003555525.1 | 3.93 | 46.5 |
| Bacillus velezensis | ZK-3 | GCA_023093915.1 | 3.92 | 46.5 |
| Bacillus velezensis | ZL918 | GCA_002157265.1 | 3.92 | 46.5 |
| | | Average = | 4.01 | 46.4 |
| | | St. Deviation = | 0.12 | 0.2 |

TABLE 7
Annotation and comparison of putative proteins encoded within the
phosphonate/phosphinate biosynthetic gene cluster of M. abscessus subsp. massiliense AA3 and
B. velezensis NRRL B-41850.

| | M. abscessus subsp. massiliense AA3 | | Percent | B. velezensis NRRL B-41850 | |
|---|---|---|---|---|---|
| ORF | Accession | NCBI Annotation | Identity | Accession | PFam Annotation |
| orf1 | SLC11717 | Putative secreted protein | 99.2 | WP_053285257 | Protein of unknown |
| | | | | | function DUF58 |
| orf2 | SLC11728 | Uncharacterized protein | 99.9 | WP_053285256 | Transglutaminase- |
| | | involved in cytokinesis | | | like superfamily |
| orf3 | SLC11771 | GMP synthase | 100 | WP_014417088 | Glutamine |
| | | | | | amidotransferase |
| | | | | | class-I |
| orf4 | SLC11800 | Biotin carboxylase | 99.5 | WP_053285255 | ATP-grasp domain |
| orf5 | SLC11817 | Biotin carboxylase | 99.5 | WP_053285254 | ATP-grasp domain |
| orf6 | SLC11840 | Isocitrate lyase family | 99.0 | WP_053285253 | Phosphoenolpyruvate |
| | | protein | | | phosphomutase |
| orf7 | SLC11886 | Aspartate/tyrosine/aromatic | 99.5 | WP_053285252 | Aminotransferase |
| | | aminotransferase | | | class I and II |
| orf8 | SLC11928 | ABC transporter permease | 97.1 | WP_053285251 | Major facilitator |
| | | | | | superfamily |
| orf9 | SLC11953 | RNA polymerase sigma | 98.9 | WP_014417094 | Sigma-70, region 4 |
| | | factor | | | |
| orf10 | SLC11990 | Uncharacterized protein | 99.2 | WP_053285250 | Domain of unknown |
| | | | | | function |
| orf11 | SLC12013 | Putative | 99.8 | WP_007408891 | Permease family |
| | | hypoxanthine/guanine | | | |
| | | permease | | | |
| orf12 | SLC12048 | Uncharacterized protein | 98.9 | WP_053285249 | — |

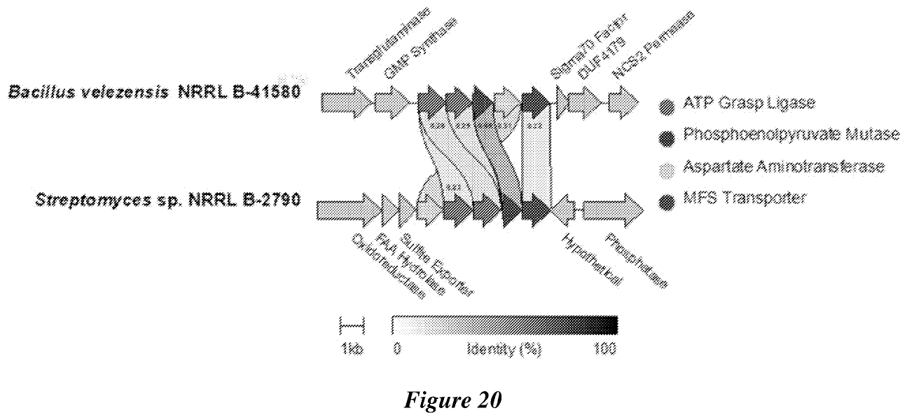

Next, the genes encoded within the pepM neighborhood were analyzed to gain insight into the potential structure and function of the phosphonate/phosphinate natural products. Intriguingly, homologs for orf4-8 (ATP-Grasp ligases, phosphoenolpyruvate mutase, aminotransferase, MFS transporter) are also present within the phosphonoalamide biosynthetic gene cluster of Streptomyces sp. NRRL B-2790 (FIG. 20). Based on these observations, it was hypothesized that NRRL B-41580 may be a new source of phosphonoalanine-containing phosphonopeptides. However, differences in putative modification enzymes surrounding these five core genes and low sequence identity between the ATP-grasp ligases suggested the phosphonate/phosphinate natural products from B. velezensis would be distinct from those produced by Streptomyces.

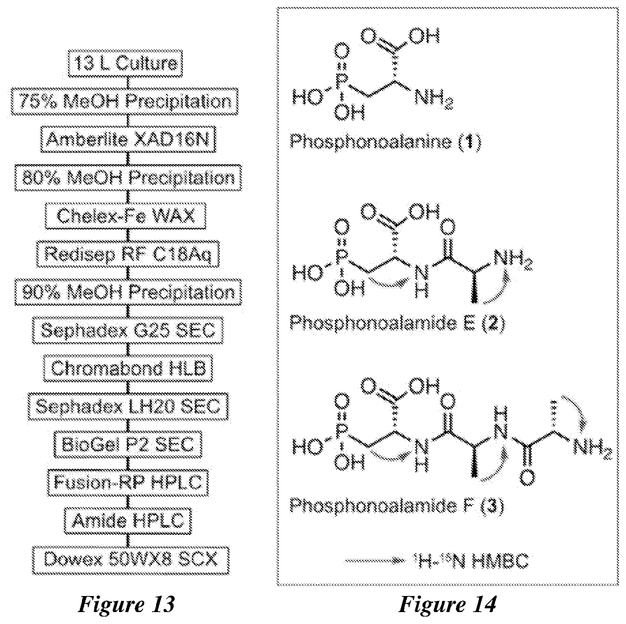

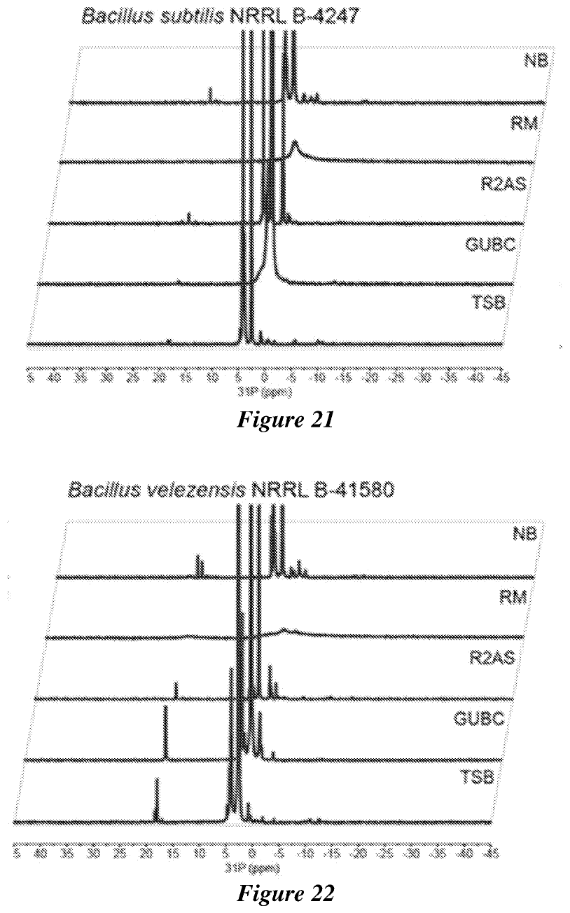

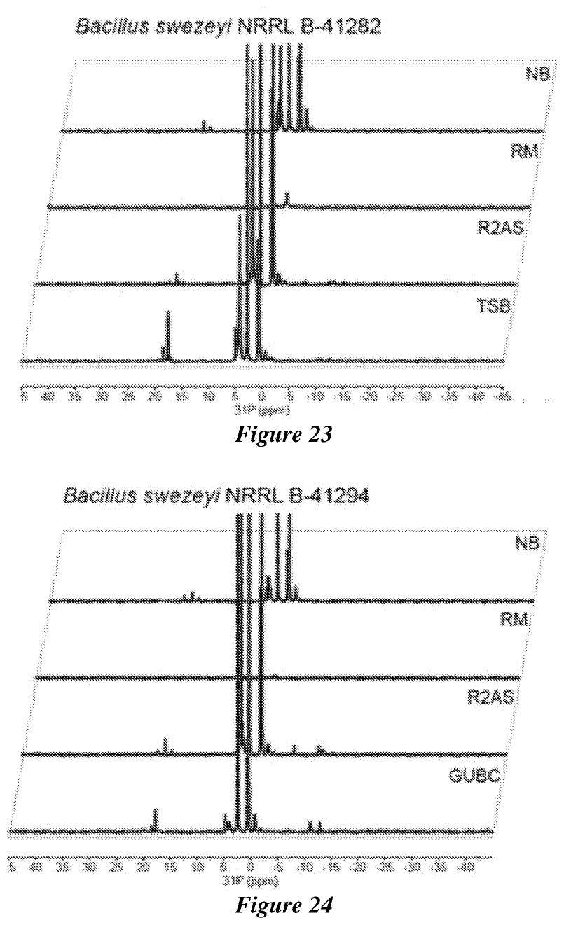

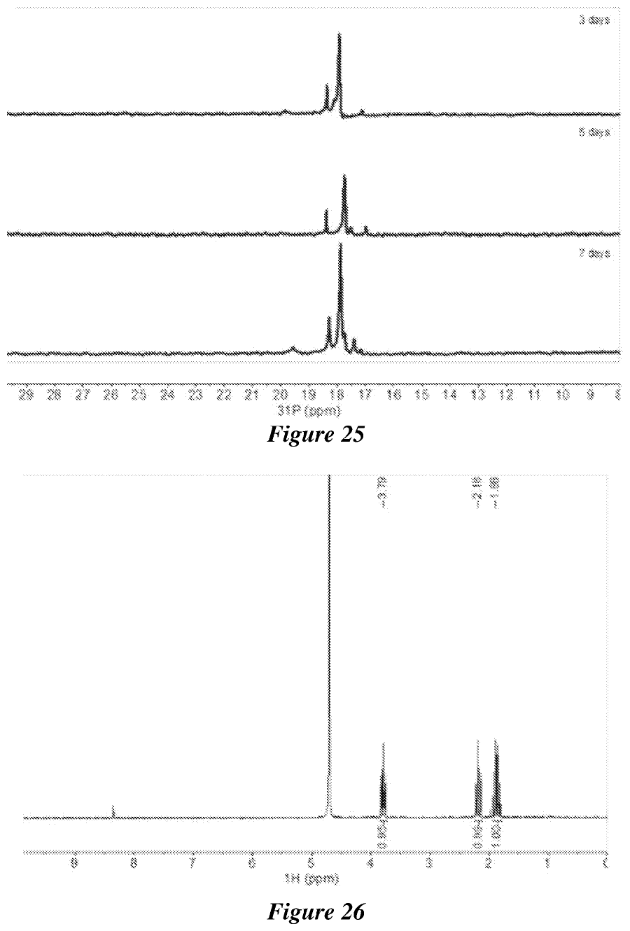

Production screening and purification. Having established the origin of the phosphonate/phosphinate biosynthetic genes, NRRL B-41580 and three additional strains encoding the same biosynthetic gene cluster (B. velezensis NRRL B-4257, B. swezeyi NRRL B-41282, and B. swezeyi NRRL B-41294) were cultured in several different media to determine the optimal conditions for phosphonate production. The resulting extracts were concentrated and analyzed by ³¹P NMR. Signals corresponding to putative phosphonate/phosphinate natural products were readily detected in all but one growth condition (FIG. 21-FIG. 24). Growth in TSB yielded the greatest number and titer of the putative phosphonates/phosphinates, with NRRL B-41580 accumulating the maximal amount on day 7 (FIG. 25). Based these results, 13 L of NRRL B-41580 culture was prepared for ³¹P NMR guided purification of the phosphonate/phosphinate compounds (FIG. 13).

FIG. 13-FIG. 16 illustrate the purification and structure elucidation for phosphonoalanine (1), phosphonoalamide E (2), and phosphonoalamide F (3).

The supernatant was combined with methanolic extracts of the cell pellets (500 mL) and concentrated to 1 L by rotary evaporation. Methanol (MeOH) was added (75% v/v) to the crude extract and incubated at −20° C. to remove undesired components through bulk precipitation. The soluble fraction was concentrated by rotary evaporation to 500 mL and incubated overnight with Amberlite XAD16 resin (200 g) at 16° C. with gentle agitation. Metabolites were sequentially eluted with water dIH₂O (750 mL) and MeOH (3 vol 750 mL). Phosphonate/phosphinate-containing fractions were combined and subjected to another round of MeOH precipitation (80% v/v) and reconcentrated to 450 mL. Weak-anion exchange chromatography (WAX) using iron-chelated Chelex-100 resin was performed, followed by reverse phase medium-pressure flash chromatography, both as previously described [A20]. The resulting phosphonate/phosphinate-containing fractions were combined, concentrated to 20 mL, precipitated with MeOH (90% v/v) and reconstituted in 30 mL of dI H₂O.

At this point the sample was highly enriched in phosphonate/phosphinate compounds, but contained significant amounts of interfering salts. To remove them, the sample was first fractionated over a Sephadex G25 size-exclusion column (5×100 cm, 1.9 L bed; dIH₂O at 1 mL min-1, 10 mL fractions). Phosphonate/phosphinate-containing fractions were combined, concentrated, and applied to a Chromabond HLB column and eluted stepwise with MeOH (2.5×10 cm, 20 mL bed; 100 mL of 0, 25, 50, 75, 100% MeOH at 1 mL min-1, 20 mL fractions). Fractions with phosphonates/phosphinates were combined and concentrated to 30 mL. This was then successively purified over two additional size exclusion chromatography (SEC) columns. The first was a Sephadex LH-20 column (2.5×180 cm, 1.2 L bed; dIH₂O at 1 mL min-1; 10 mL fractions), and the second a BioGel P2 column (2.5×100 cm, 600 mL bed; dIH₂O at 0.5 mL min⁻¹, 3 mL fractions).

Following fractionation, the sample was lyophilized and dissolved in dIH₂O for purification by HPLC. This was first separated using a Phenomenex Fusion-RP column (10×250 cm) using a gradient program with dIH₂O (A) and MeOH (B) (0-10 min 100% A, 10-45 min linear gradient to 70% B, 45-46 min linear gradient to 100% B, 46-55 min 100% B, 55-75 min 100% A; 3 mL min⁻¹, 3 mL fractions). The phosphonates/phosphinates were not retained on the column, indicating a high degree of hydrophilicity. Nonetheless, this step further reduced chemical complexity by removing all remaining hydrophobic compounds. The phosphonates/phosphinates were then dissolved in 70% acetonitrile (MeCN) and separated on a Waters BEH Amide column (10×250 mm) using a gradient program with dIH₂O (A) and MeCN (B) containing 0.1% formic acid (FA) (0-5 min 70% B, 5-35 min linear gradient to 40% B, 35-45 min 40% B, 45-50 min linear gradient to 70% B, 50-70 min 70% B; 4 mL min⁻¹, 4 mL fractions). The phosphonate/phosphinate-containing fractions (12-14 min) were combined, reconstituted in 75% MeCN, and separated using an isocratic method with the same column (75% MeCN with 0.1% FA; 4 mL min⁻¹, 4 mL fractions). This yielded 25 mg of purified compound 3, but compounds 1 and 2 remained unresolved from each other. Fractions containing the latter were lyophilized, dissolved in 2 mL dIH₂O, acidified to pH 3 with FA, and separated by strong cation exchange (SCX) using Dowex 50W-X8 (1×10 cm, 5 mL bed; 2 mL min-1; 2.5 mL fractions). The column was eluted first with dIH₂O (50 mL) and then 0.1% FA (25 mL). Water fractions containing phosphonates/phosphinates were combined to yield 0.8 mg of pure compound 1. FA fractions containing phosphonates/phosphinates combined to yield 0.8 mg of pure compound 2.

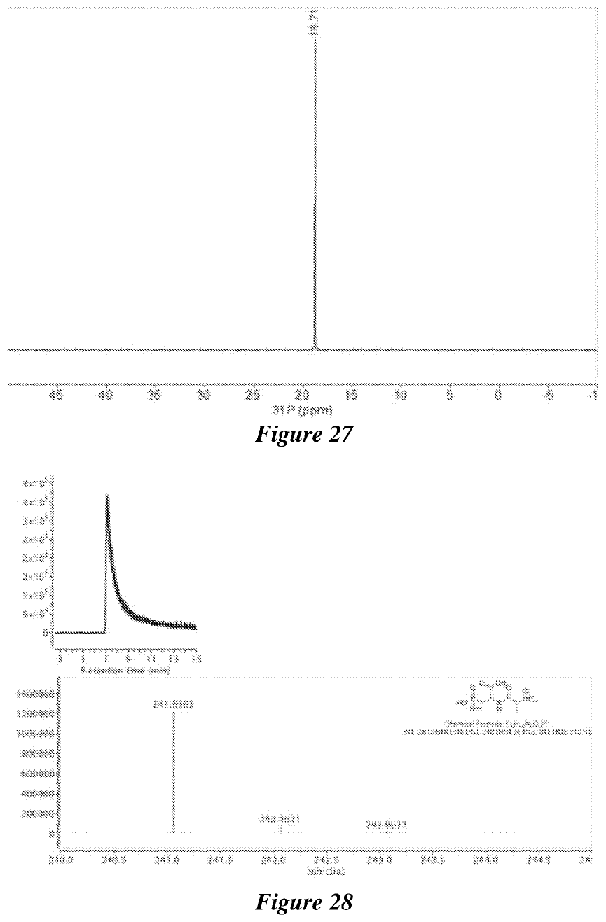

Structure Elucidation. Compound 1 was isolated as a white amorphous solid. Its molecular formula was deduced as C₃H₉NO₅P⁺ from HRMS analysis ([M+H]⁺ calcd m/z 170.0213, observed m/z 170.0212; Δppm=−0.6). These values and the ¹H and ³¹P NMR data were identical to literature values of phosphonoalanine (FIG. 26-FIG. 27; Table 8) [A37]. Thus, 1 was identified as L-phosphonoalanine (FIG. 14).

TABLE 8
¹H (400 MHz) and ³¹P (162 MHz) spectroscopic
data for compound 1 in 100% D₂O.

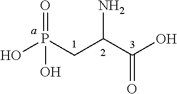

| No. | δ_{H} (J in Hz) | δ_{P} |
|---|---|---|
| 1 | 1.88, td (14.5, 11.3, 0.7) 2.18 m | — |
| 2 | 3.79, m | — |
| 3 | — | — |
| a | — | 18.71 |

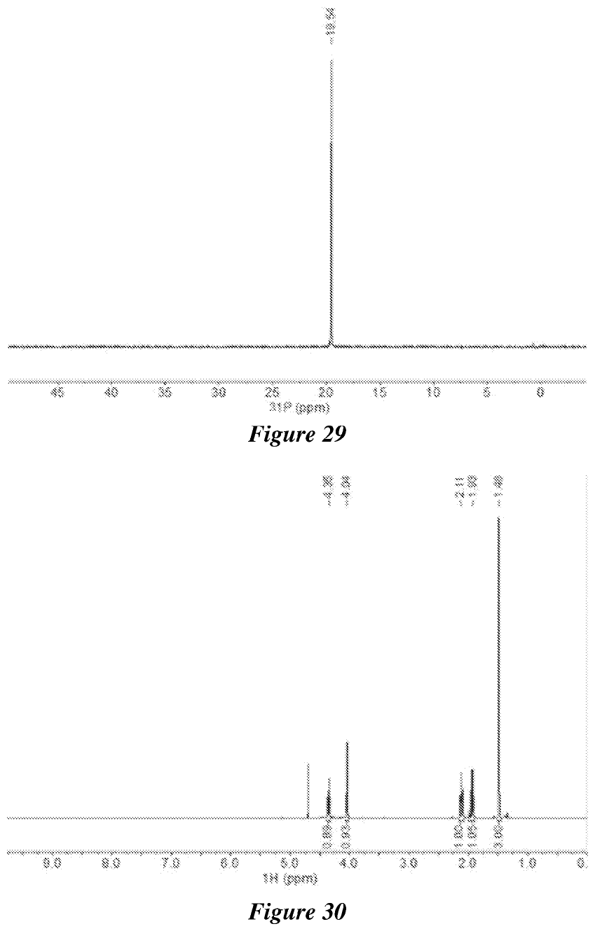

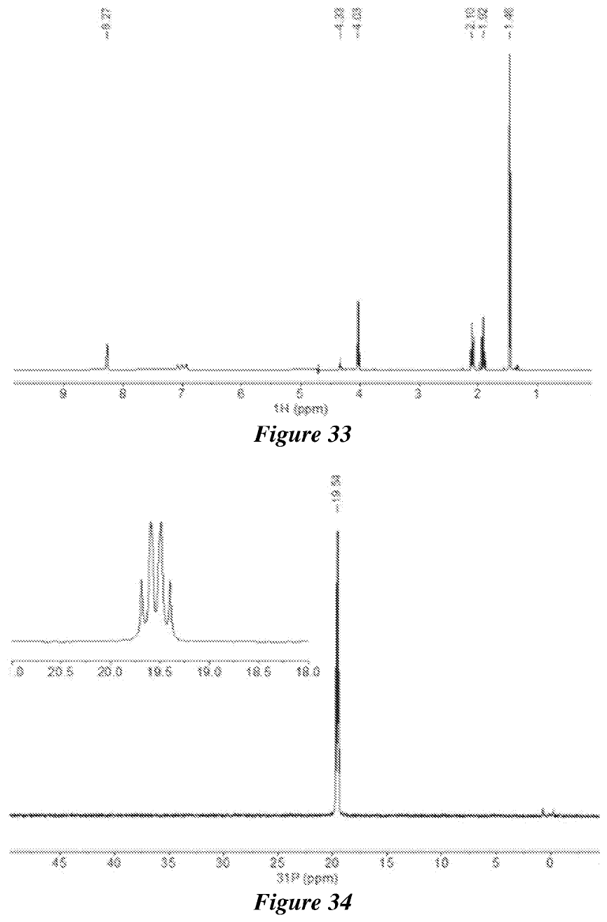

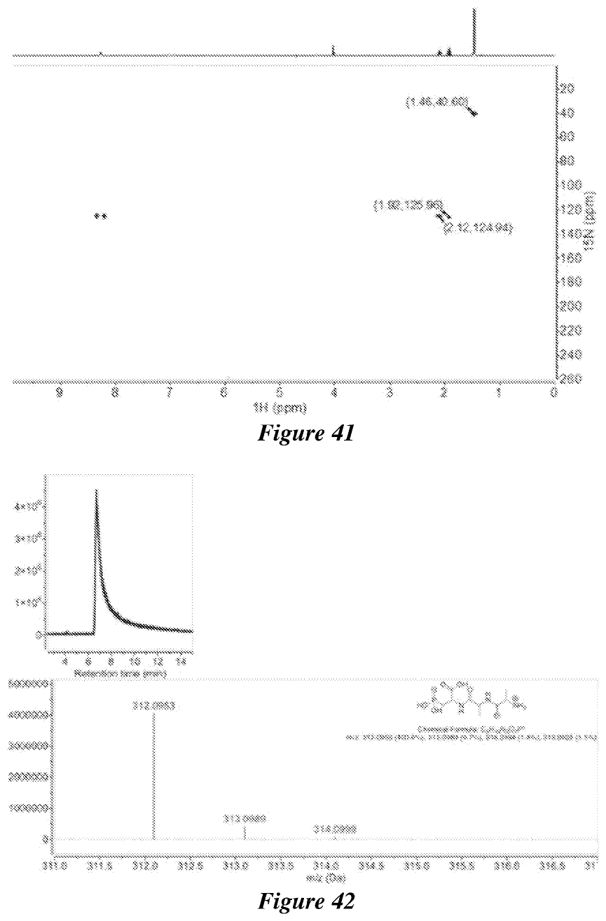

Compound 2 was obtained as a white amorphous solid. Its molecular formula was deduced as C₆H₁₄N₂O₆P⁺ from HRMS analysis ([M+H]⁺ calcd m/z 241.0584, observed m/z 241.0583; Δppm=−0.4; FIG. 28). A series of 1D, 2D homonuclear, and 2D heteronuclear NMR experiments were performed to elucidate the chemical structure of this compound (FIG. 29-FIG. 41). The ³¹P NMR spectrum showed a single resonance at 19.54 ppm (FIG. 29). The ¹H spectrum showed resonances for 3 methyl protons (H-3′; δ_{H} 1.46), 2 methylene protons (H-1; 8H 1.93, 2.11), and 2 methine protons (H-2′, H-2; δ_{H} 4.04, 4.35) (FIG. 30). These were corroborated by ¹³C DEPT 135 and multiplicity-edited ¹H-¹³C HSQC (FIG. 32, FIG. 38) experiments. Analysis in 90% H₂O/10% D₂O revealed one additional amide proton undergoing solvent exchange (H-b; δ_{H} 8.27) (FIG. 33). ¹H-¹⁵N HSQC and ¹H-¹⁵N HMBC confirmed H-b was indeed directly attached to an amide nitrogen atom (N-b; δ_{N} 124.96) (FIG. 40-FIG. 41).

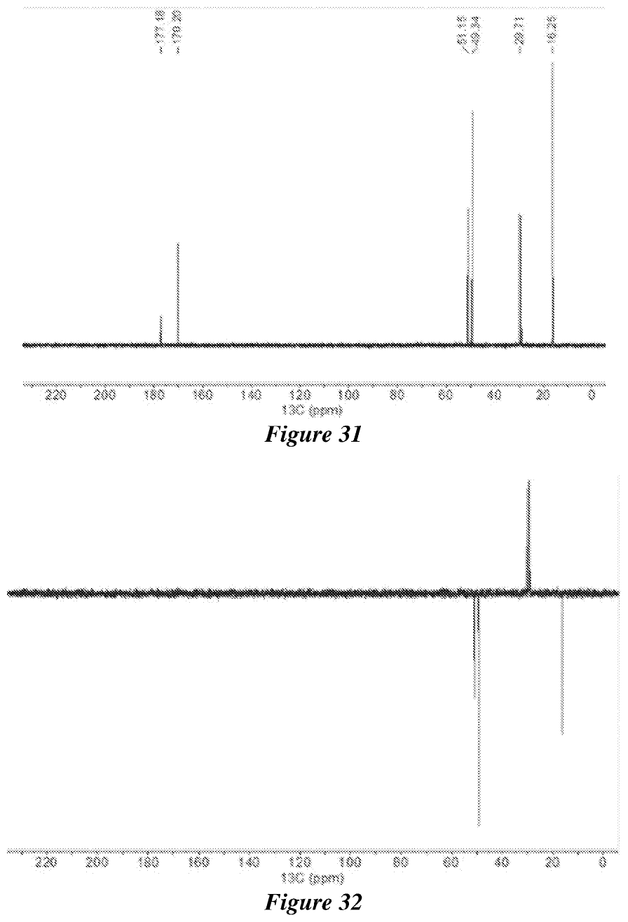

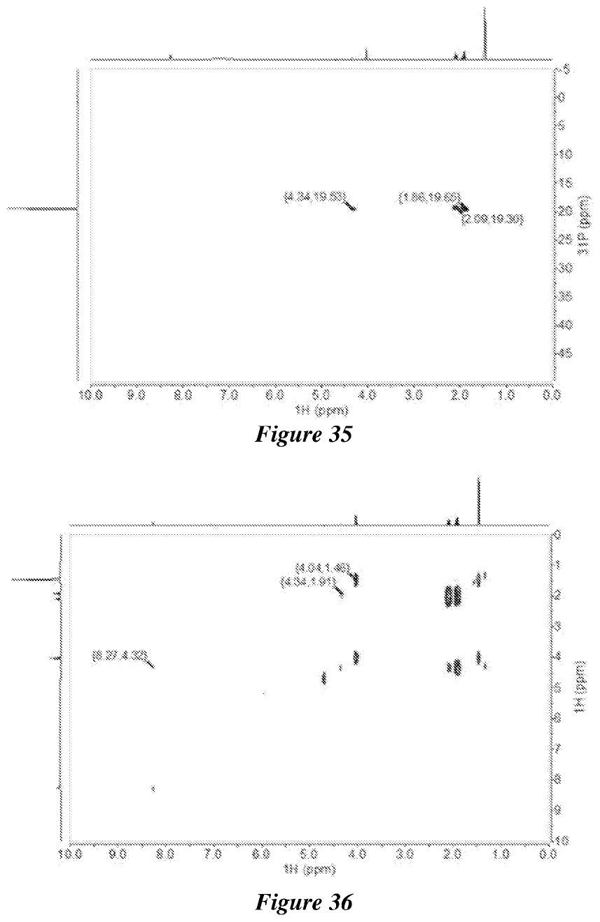

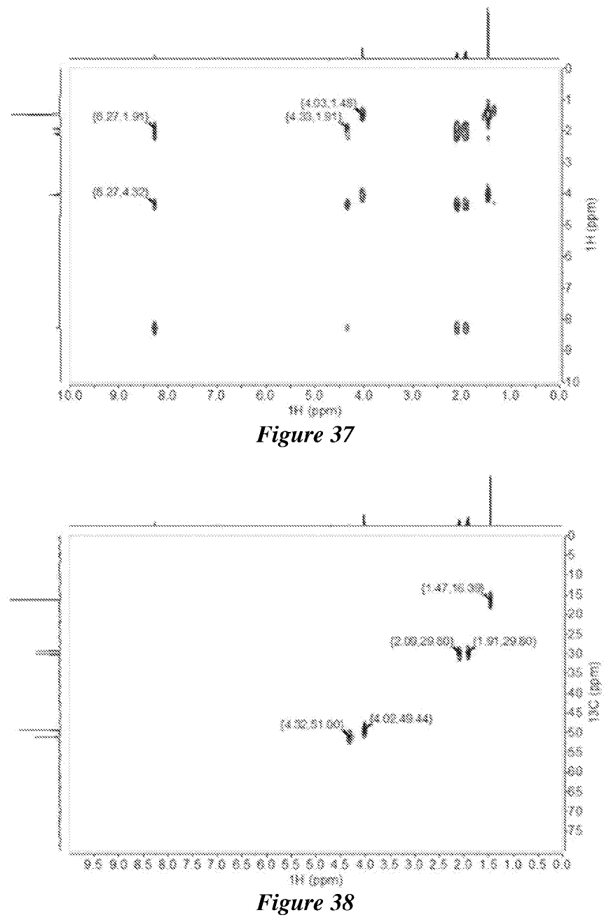

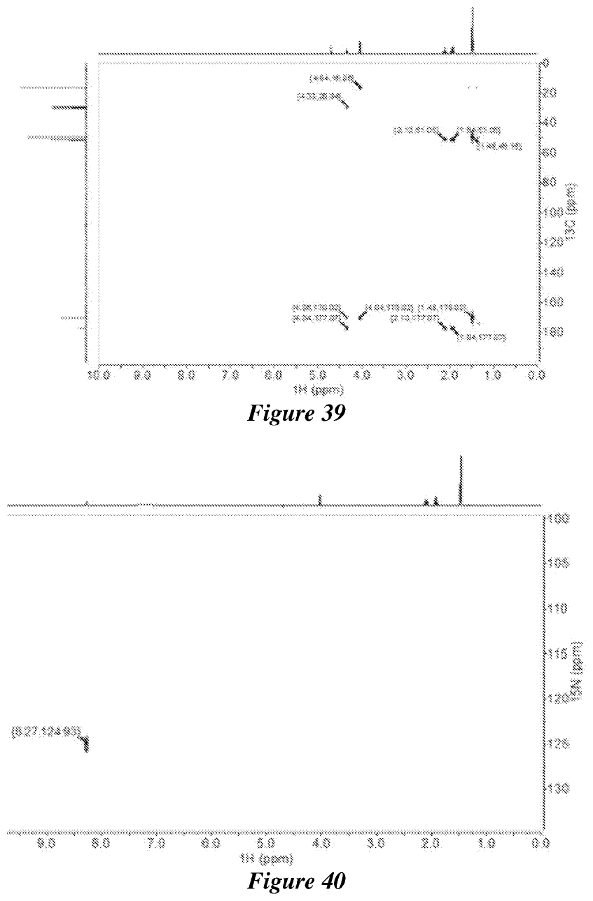

¹H-³¹P HMBC demonstrated correlation of methylene H-1 and methine H-2 protons with the phosphorous atom (Op 19.54) in a pattern consistent with phosphonoalanine (FIG. 35). ¹H-¹³C HSQC and ¹H-¹³C HMBC data then established direct attachment of H-1 with secondary carbon C-1 (8c 29.71), H-2 with tertiary carbon C-2 (8c 51.14), and C-2 that is connected to quaternary carboxylate carbon C-3 (8c 177.18) (FIG. 38-FIG. 39). ¹H-¹H COSY and TOCSY further verified H₁, H-2, and H-b were in the same spin system and indicated the amide moiety connected to C-2 (FIG. 36-FIG. 37). Lastly, diagnostic splitting and J_{CP} coupling constants observed in the ¹³C spectrum confirmed the C—P bond (FIG. 31; Table 9). These data indicate phosphonoalanine was a component of 2.

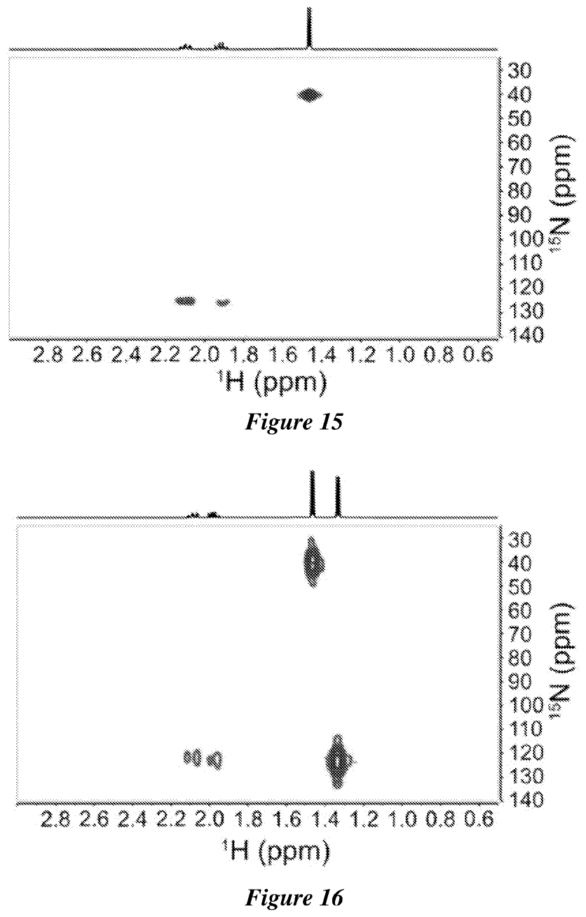

The second isolated spin system in the ¹H-¹H COSY and ¹H-¹H TOCSY spectra was consistent with alanine (H-2′, δ_{H} 4.03; H-3′, δ_{H} 1.46) (FIG. 36-FIG. 37). This was corroborated by ¹³C DEPT 135, multiplicity-edited ¹H-¹³C HSQC, and ¹H-¹³C HMBC spectra (FIG. 32, FIG. 38, FIG. 39). Observation of the free amine nitrogen (N-c; δ_{N} 40.60) of alanine in ¹H-¹⁵N HMBC spectra (FIG. 15; FIG. 41), in conjunction with the above data, confirmed its direct attachment to the a carbon (C-2′, δ_{C} 49.34). The correlation between methyl protons of the alanyl side chain (H-3′; δ_{H} 1.48) and N-c further supported this as the free amine. Thus, the second half of 2 was composed of alanine.

Connectivity between the phosphonoalanine and alanine was established with the ¹H-¹³C HMBC dataset (FIG. 39). Specifically, correlation between the methine proton of phosphonoalanine (H-2, δ_{H} 4.33) with the carbonyl carbon of alanine (C-2′, δ_{C} 49.34) spanned the amide moiety of phosphonoalanine. Thus, 2 is a dipeptide composed of _{L}-alanine-_{L}-phosphonoalanine. All NMR data are consistent with this structure (FIG. 29-FIG. 41; Table 9). This compound was phosphonoalamide E (FIG. 14).

TABLE 9
¹H (700 MHz), ¹³C (176 MHz), ³¹P (162 MHz), and ¹⁵N (71 MHz) spectroscopic data for compound 2.

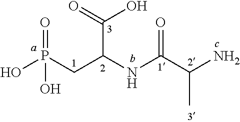

| | δ_{C}, mult | δ_{H}, mult | δ_{P}, mult | δ_{N}, mult | ¹H-¹H | ¹H-¹H | ¹H-¹³C | ¹H-³¹P | ¹H-¹⁵N |
|---|---|---|---|---|---|---|---|---|---|
| No. | (J in Hz)¹ | (J in Hz)¹ | (J in Hz)¹ | (J in Hz)¹ | COSY¹ | TOCSY¹ | HMBC² | HMBC¹ | HMBC¹ |
| 1 | 29.71, d | 1.92, td | — | — | 2 | 2, b | 2, 3 | | b |
| | (133.1) | (15.2, | | | | | | | |
| | | 10.7) | | | | | | | |
| | | 2.10, m | | | | | | | |
| 2 | 51.14, d | 4.33, m | — | — | 1 | 1,b | 1, 3, 1′ | | — |
| | (5.27) | | | | | | | | |
| 3 | 177.18, d | — | — | — | — | — | — | — | — |
| | (14.85) | | | | | | | | |
| 1′ | 170.20, s | — | — | — | — | — | — | — | — |
| 2′ | 49.34, s | 4.03, q | — | — | 3′ | 3′ | 1′, 3′ | — | — |
| | | (7.1) | | | | | | | |
| 3′ | 16.25, s | 1.46, d | — | — | 2′ | 2′ | 1′, 2′ | — | c |
| | | (7.2) | | | | | | | |
| a | — | — | 19.54 | — | — | — | — | — | — |
| b | — | 8.27, d | — | 124.96 | — | 1, 2 | — | — | — |
| | | (6.9) | | | | | | | |
| c | — | — | — | 40.60 | — | — | — | — | — |

¹Collected in 90% H₂O/10% D₂O
²Collected in 100% D₂O

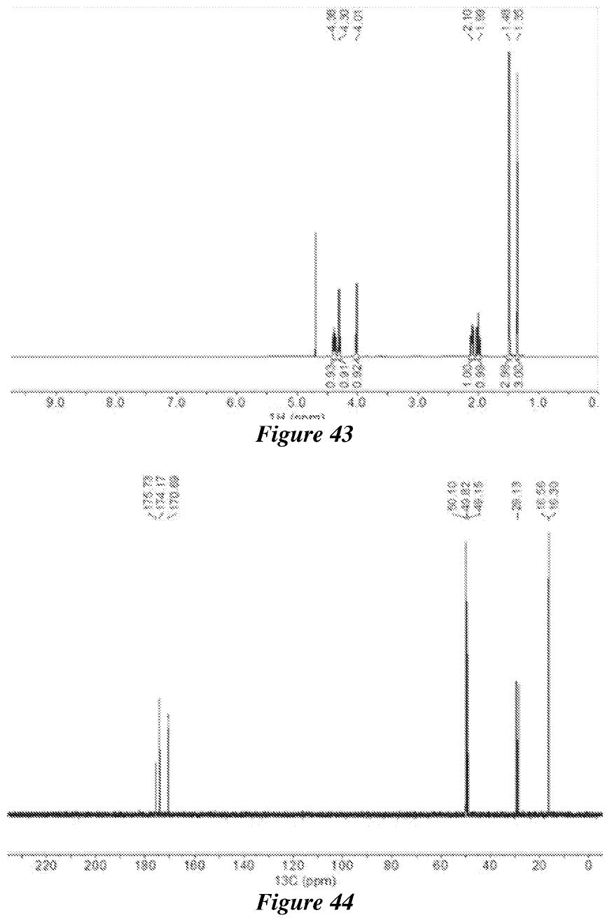

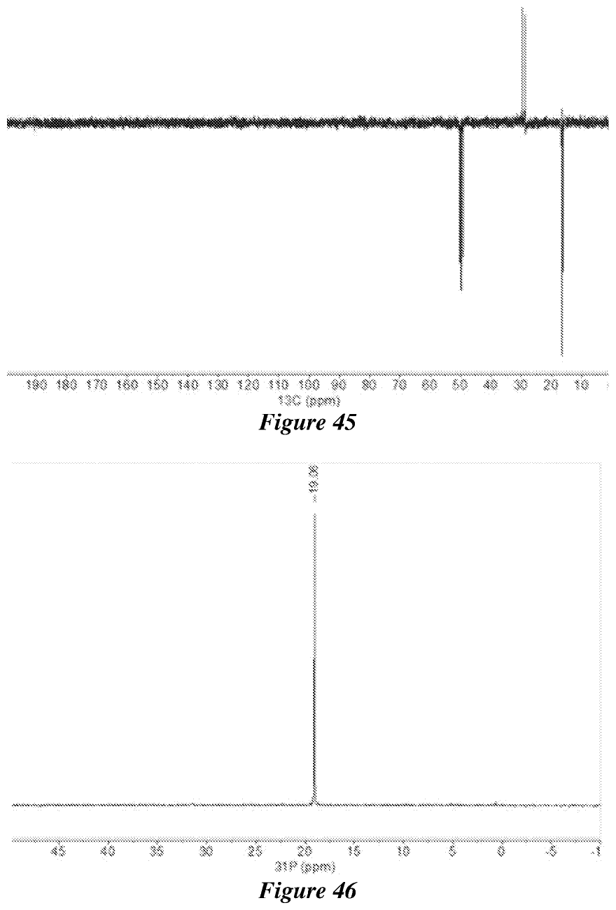

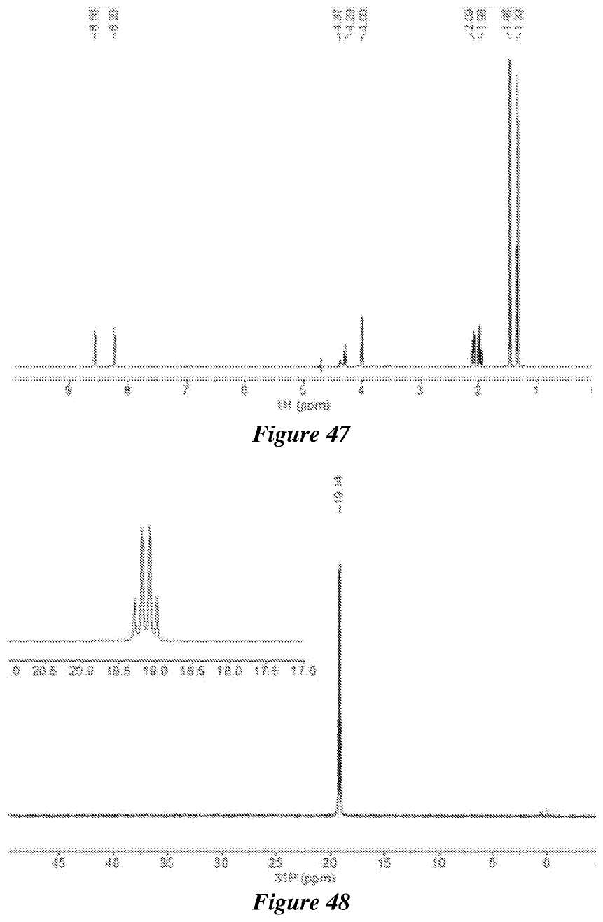

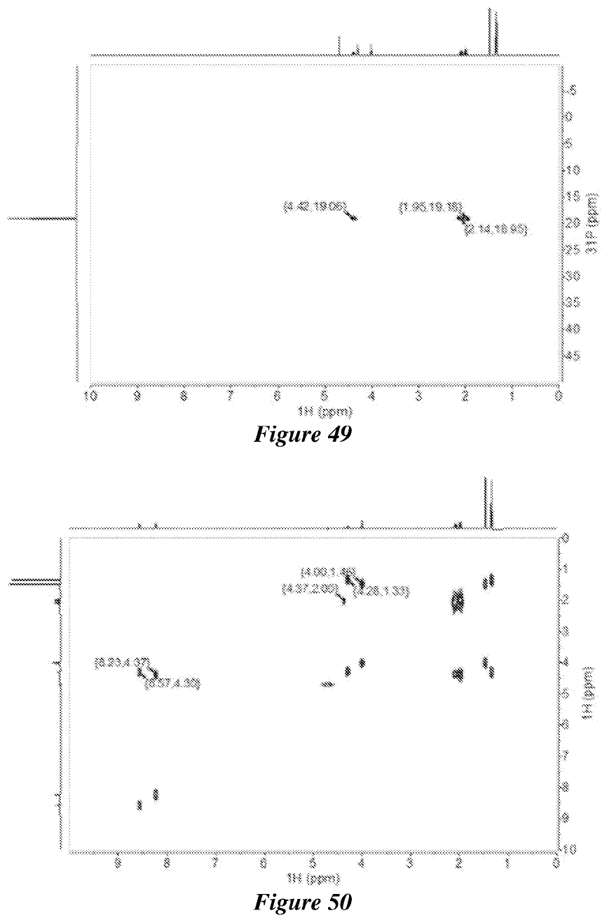

Compound 3 was obtained as a white amorphous solid. Its molecular formula was deduced as C₉H₁₉N₃O₇P⁺ from HRMS analysis ([M+H]⁺ calcd m/z 312.0955, observed m/z 312.0952; Δppm=−0.9; FIG. 42). As above 1D, 2D homonuclear, and 2D heteronuclear NMR experiments were used to elucidate its chemical structure (FIG. 43-FIG. 55). The ¹H spectrum showed resonances for 13 protons (FIG. 43). Multiplicity edited ¹H-¹³C HSQC verified them as 6 methyl (H-3′, H-3″; OH 1.33, 1.46). 2 methylene (H-1; δ_{H} 1.98, 2.09), and 3 methine protons (H-2″, H-2′, H-2; δ_{H} 4.00, 4.29, 4.37) (FIG. 52). The final two amide protons (H-b, H-c; δ_{H} 8.23, 8.57) were observed to undergo solvent exchange (FIG. 47). ¹H-¹⁵N HSQC confirmed their direct connection to nitrogen atoms (N-b, N-c; δ_{N} 122.00, 123.49) (FIG. 54). As with 2, ¹H, ¹H-³¹ P HMBC, ¹H-¹H COSY, ¹H-¹H TOCSY, ¹H-¹³C HSQC, and ¹H-¹³C HMBC data indicated 3 contained phosphonoalanine, but with additional constituents attached at N-b (FIG. 43, FIG. 50-FIG. 53).

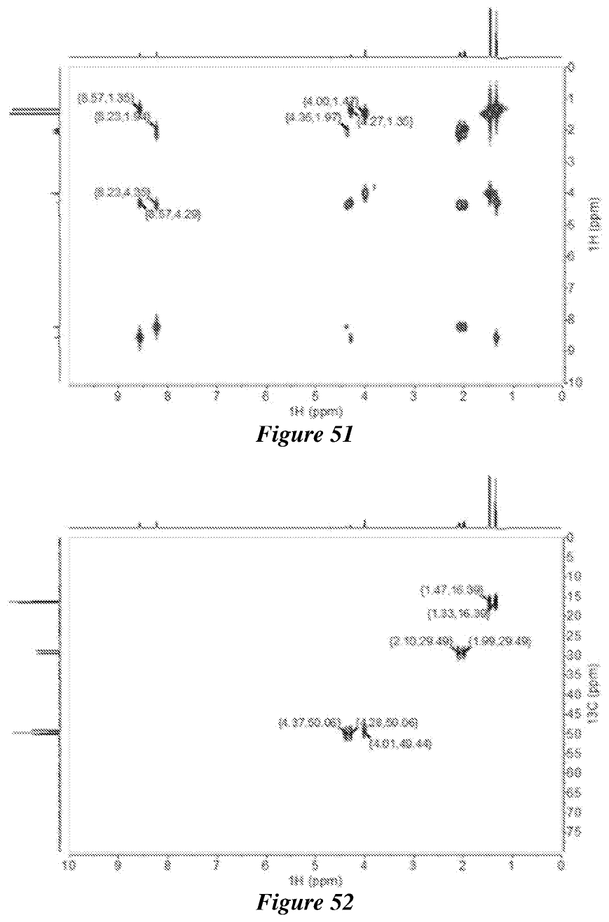

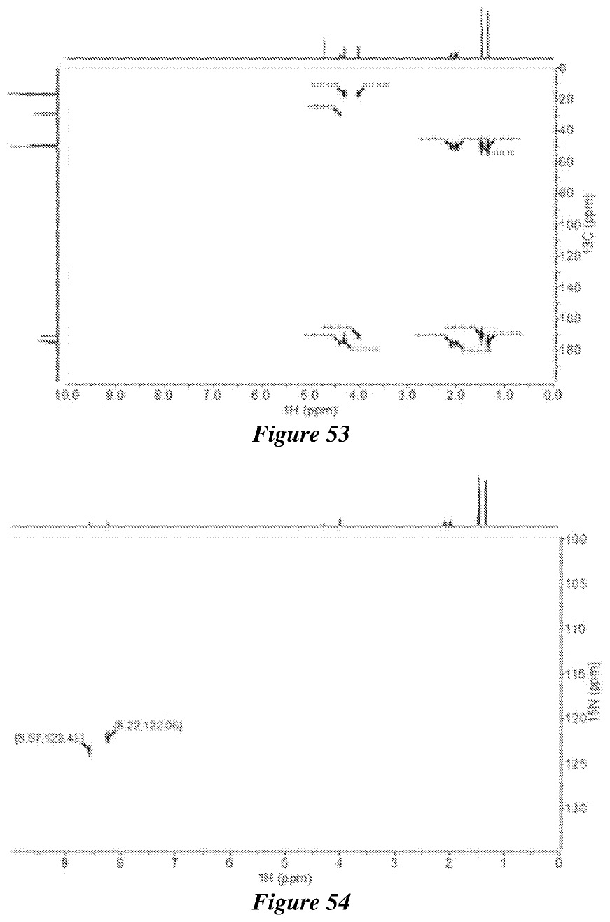

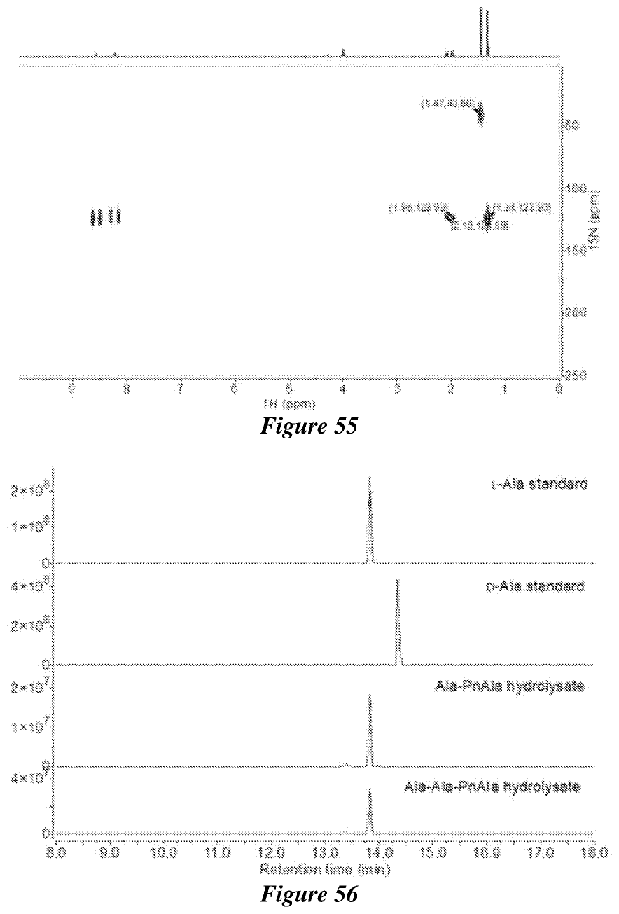

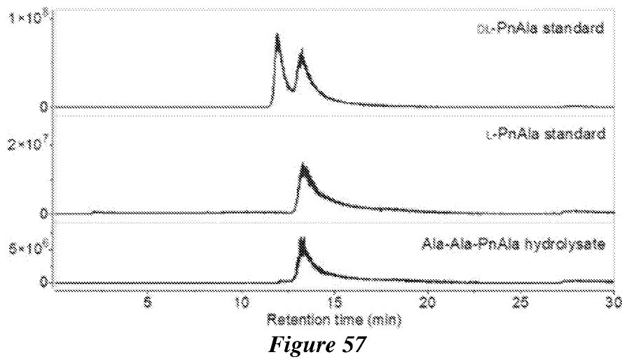

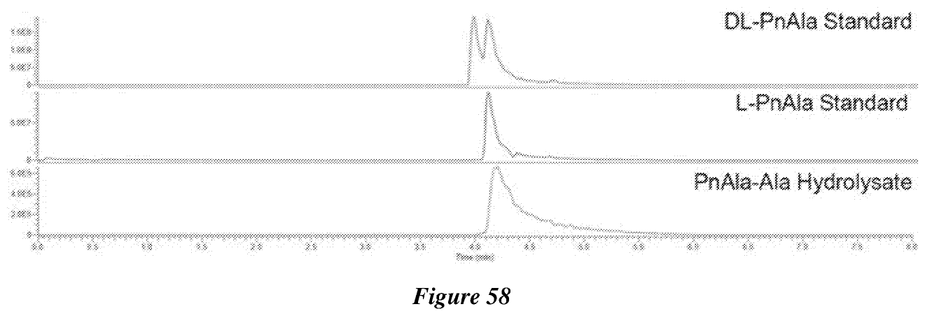

The observation of two amide protons (H-b, H-c; δ_{H} 8.23, 8.57) and two carbonyl carbons (C-1′, C-1″; δ_{C} 174.17, 170.69) indicated the potential ligation of a dipeptide to phosphonoalanine (FIG. 44, FIG. 47). Two additional spin systems within ¹H-¹H COSY and ¹H-¹H TOCSY datasets showed that 3 was composed of two alanine residues (FIG. 50-FIG. 51). Placement of the alanine-alanine amide bond was confirmed by correlation between methine proton H-2′ and carbonyl carbon C-1″ (δ_{C} 170.69) in the ¹H-¹³C HMBC dataset (FIG. 53). Correlation between the methyl group of the terminal alanine (H-3″; δ_{H} 1.46) and the amine of the terminal alanine (N-d; δ_{N} 40.22) in the ¹H-¹⁵N HMBC dataset established alanine at the N-terminus of 3 (FIG. 16; FIG. 55). The absolute configuration of all three amino acids was determined as L by Marfey's analysis (FIG. 56-FIG. 58). All NMR data are consistent with the structure of 3 (FIG. 42-FIG. 55; Table 10) as the tripeptide _{L}-alanine-_{L}-alanine-_{L}-phosphonoalanine. This compound was named phosphonoalamide F (FIG. 14).

Table 11 summarizes all of the 2D NMR correlations observed from homo- and heteronuclear experiments.

TABLE 10
¹H (700 MHz), ¹³C (176 MHz), ³¹P (162 MHz), and ¹⁵N (71 MHz) spectroscopic data for compound 3.

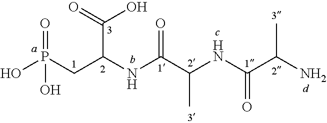 [SMILES: CC(N)C(=O)NC(C)C(=O)NC(CP(=O)(O)O)C(=O)O]

| | δ_{C}, mult | δ_{H}, mult | δ_{P}, mult | δ_{N}, mult | ¹H-¹H | ¹H-¹H | ¹H-¹³C | ¹H-³¹P | ¹H-¹⁵N |
|---|---|---|---|---|---|---|---|---|---|
| No. | (J in Hz)¹ | (J in Hz)¹ | (J in Hz)¹ | (J in Hz)¹ | COSY¹ | TOCSY¹ | HMBC² | HMBC² | HMBC¹ |
| 1 | 29.13, d | 1.98, td | — | — | 2 | 2, b | 2, 3 | a | b |
| | (132.6) | (8.9, | | | | | | | |
| | | 15.6, | | | | | | | |
| | | 15.4) | | | | | | | |
| | | 2.09, ddd | | | | | | | |
| | | (5.0, | | | | | | | |
| | | 15.3, | | | | | | | |
| | | 17.6) | | | | | | | |
| 2 | 50.10, d | 4.37, m | — | — | 1 | 1, b | 1,3 | a | — |
| | (4.6) | | | | | | | | |
| 3 | 175.73, d | — | — | — | — | — | — | — | — |
| | (12.5) | | | | | | | | |
| 1′ | 174.17, s | — | — | — | — | — | — | — | — |
| 2′ | 49.82, s | 4.29, p | — | — | 3′, c | 3′, c | 3′, 1″ | — | — |
| | | (7.1) | | | | | | | |
| 3′ | 16.30, s | 1.33, d | — | — | 2′ | 2′, c | 1′, 2′ | — | c |
| | | (7.2) | | | | | | | |
| 1″ | 170.69, s | — | — | — | — | — | — | — | — |
| 2″ | 49.15, s | 4.00, q | — | — | 3″ | 3″ | 1″, 3″ | — | — |
| | | (7.1) | | | | | | | |
| 3″ | 16.56, s | 1.46, d | — | — | 2″ | 2″ | 1″, 2″ | — | d |
| | | (7.1) | | | | | | | |
| a | — | — | 19.13, q | — | — | — | — | — | — |
| | | | (16.4) | | | | | | |
| b | — | 8.23, d | — | 122.00 | — | 1, 2 | — | — | — |
| | | (6.5) | | | | | | | |
| c | — | 8.55, d | — | 123.49 | 2′ | 2′, 3′ | — | — | — |
| | | (6.0) | | | | | | | |
| d | — | — | — | 40.22 | — | — | — | — | — |

¹Collected in 90% H₂O/10% D₂O
²Collected in 100% D₂O

TABLE 11
Summary of 2D NMR correlations observed for compounds 2 and 3

| Experiment | Phosphonoalamide E (2) | Phosphonoalamide F (3) |
|---|---|---|
| ¹H-¹H COSY | 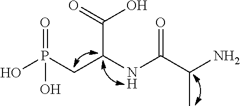 | 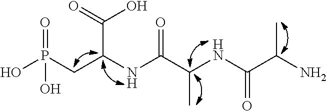 [SMILES: CC(N)C(=O)NC(C)C(=O)NC(CP(=O)(O)O)C(=O)O] |
| ¹H-¹H TOCSY | 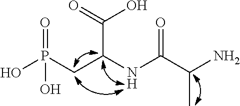 | 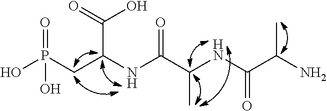 [SMILES: CC(N)C(=O)NC(C)C(=O)NC(CP(=O)(O)O)C(=O)O] |
| ¹H-¹³C HMBC | 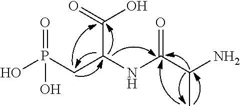 | 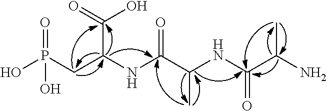 [SMILES: CC(N)C(=O)NC(C)C(=O)NC(CP(=O)(O)O)C(=O)O] |
| ¹H-³¹P HMBC | 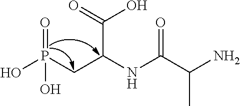 | 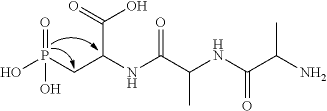 [SMILES: CC(N)C(=O)NC(C)C(=O)NC(CP(=O)(O)O)C(=O)O] |
| ¹H-¹⁵N HMBC | 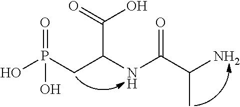 | 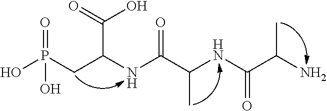 [SMILES: CC(N)C(=O)NC(C)C(=O)NC(CP(=O)(O)O)C(=O)O] |

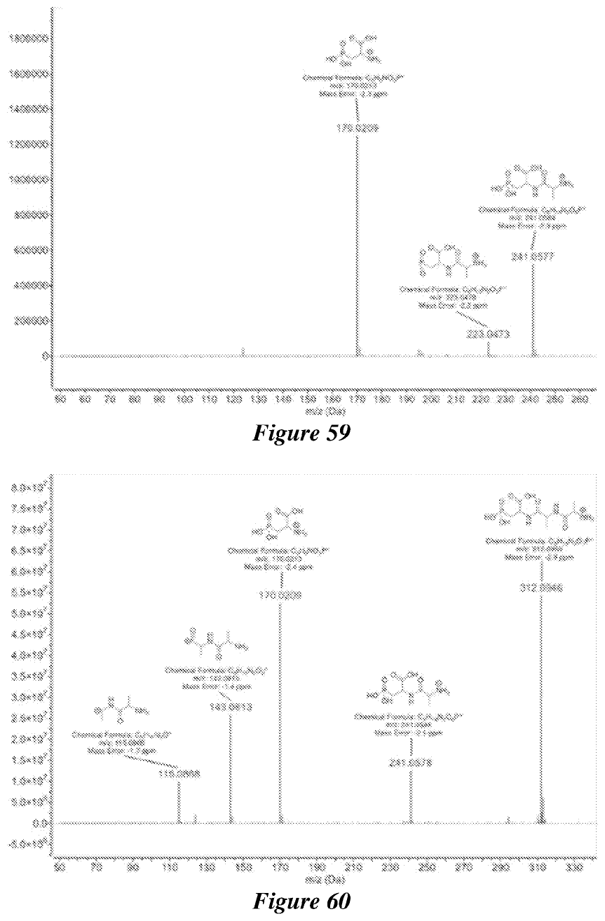

High resolution tandem MS data provided rich fragmentation that further corroborated the structures of 1-3 (FIG. 8-FIG. 10; FIG. 59-FIG. 60). Notably, ions for phosphonoalanine and a diagnostic decarboxylated product (m/z 124.0159) were observed within the MS spectra of all three compounds. An ion corresponding to alanine-phosphonoalanine (m/z 124.0159) was present in the spectra of 2 and 3. The structure of 3 was further verified by additional daughter ions generated from fragmentation at the alanine-phosphonoalanine peptide bond (m/z 143.0813), the alanine-alanine peptide bond (m/z 241.0578), and the alanine-alanine moiety (m/z 115.0868).

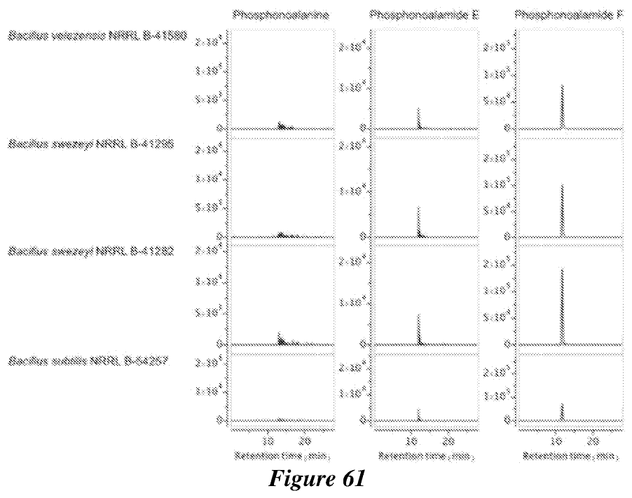

Having established the complete chemical structures of all three compounds, the initial concentrated extracts of each strain were analyze. LC-HRMS showed three compounds were indeed present, with 3 in the greatest abundance (FIG. 61). These data validate the phosphonates/phosphinates were indeed naturally produced and not artifactual derivatives (i.e. chemically modified products) generated during purification.

Antimicrobial Activity. The antimicrobial activities of 2 and 3 against a broad panel of bacteria, yeast, and filamentous fungi were determined using microbroth dilution and disk diffusion assays (Table 12). While neither compound exhibited antifungal activity, several bacterial strains were inhibited with varying degrees of efficacy (Table 13). 3 exhibited potent inhibition against enteric bacteria including E. coli, and with selectivity against Salmonella enterica LT2, but neither Tennessee nor Livingstone. 3 demonstrated selectivity against P. aeruginosa, inhibiting strain K but not PA01. Serratia marcescens was also inhibited but required a significantly higher concentration of the tripeptide. Given that B. velezensis is a common epiphyte [A38], it was hypothesized that the compounds may be effective against plant-associated bacteria. Neither compound inhibited strains of Curtobacterium, Acidovorax, and 5 Pseudomonas commonly present in soils and on plants. However, strong inhibition was also observed when B. subtilis, B. megaterium, Erwinia rhapontici, Pantoea ananatis, and Paenibacillus larvae were challenged with 3. In general, 2 demonstrated reduced susceptibility and potency against the assayed strains.

TABLE 12
Bioassay strains used in this study
Strains
Enterobacterales
Citrobacter freundii ATCC 8090
Enterobacter aerogenes CDC 1998-68
Escherichia coli K12
Salmonella enterica LT2
Salmonella enterica ser. Tennessee E2007000304
Salmonella enterica ser. Livingstone 1236H
Serratia marcescens NRRL B-2544
Pantoea ananatis LMG 20103
Erwinia rhapontici KSJ3948
Actinomycetales
Rhodococcus jostii RHA1
Mycobacterium smegmatis NRRL B-14616
Curtobacterium sp. MMLR14_014
Bacillales
Staphylococcus aureus ATCC 23055
Paenibacillus larvae NRRL B-2605
Bacillus subtilis ATCC 6633
Bacillus megaterium ATCC 19213
Pseudomonadales
Acinetobacter calcoaceticus ATCC 23055
Pseudomonas aeruginosa K
Pseudomonas aeruginosa PA01
Pseudomonas fluorescens Pf5
Pseudomonas medocina Palleroni ATCC 25411
Pseudomonas syringae B278a
Pseudomonas syringae pv. Tomato Max 14
Burkholderiales
Acidovorax avenae ATCC 19860
Acidovorax citrulli ATCC 29625
Acidovroax delafieldii ATCC 17505
Acidovorax konjacii ATCC 33996
Acidovorax temperans ATCC 49665
Yeasts
Candida albicans ATCC MYA-2876
Debaryomyces hansenii CBS-767
Saccharomyces cerevisiae KSJ4150
Schizosaccharomyces pombe ATCC 24843
Kluyveromyces lactis Y-8279
Sporobolomyces roseus KSJ4237
Filamentous fungi
Aspergillus niger ATCC 16404
Aspergillus nidulans FGSC A4
Neurospora crassa FGSC 4200
Penicillium notatum ATCC 9478

TABLE 13
Antibacterial activity of phosphonoalamides E (2) and F (3) against
strains grown in minimal media (—, not determined).

| Organism | MIC (μM) | |
|---|---|---|
| Order/Strain | 2 | 3 |
| Enterobacterales | | |
| Citrobacter freundii ATCC 8090 | >200 | >200 |
| Enterobacter aerogenes CDC 1998-68 | >200 | >200 |
| Erwinia rhapontici KSJ3948 | >200 | 6.25 |
| Escherichia coli K12 | >200 | 6.25 |
| Pantoea ananatis LMG 20103 | 100 | 3.12 |
| Salmonella enterica LT2 | >200 | 12.5 |
| Salmonella enterica ser. Tennessee E2007000304 | >200 | >200 |
| Salmonella enterica ser. Livingstone 1236H | >200 | >200 |
| Serratia marcescens NRRL B-2544 | >200 | 200 |
| Actinomycetales | | |
| Rhodococcus jostii RHA1 | >200 | >200 |
| Bacillales | | |
| Bacillus subtilis ATCC 6633 | 200 | 12.5 |
| Bacillus megaterium ATCC 19213 | 12.5 | 6.25 |
| Paenibacillus larvae NRRL B-2605 | — | 12.5 |
| Staphylococcus aureus ATCC 23055 | >200 | >200 |
| Pseudomonadales | | |
| Acinetobacter calcoaceticus ATCC 23055 | >200 | >200 |
| Pseudomonas aeruginosa K | >200 | 25 |
| Pseudomonas aeruginosa PA01 | >200 | >200 |
| Pseudomonas fluorescens Pf5 | >200 | >200 |
| Pseudomonas medocina Palleroni ATCC 25411 | >200 | >200 |
| Pseudomonas syringae B278a | >200 | >200 |
| Pseudomonas syringae pv. Tomato Max 14 | >200 | >200 |
| Burkholderiales | | |
| Acidovorax avenae ATCC 19860 | >200 | >200 |
| Acidovorax citrulli ATCC 29625 | >200 | >200 |
| Acidovroax delafieldii ATCC 17505 | >200 | >200 |
| Acidovorax konjacii ATCC 33996 | >200 | >200 |
| Acidovorax temperans ATCC 49665 | >200 | >200 |

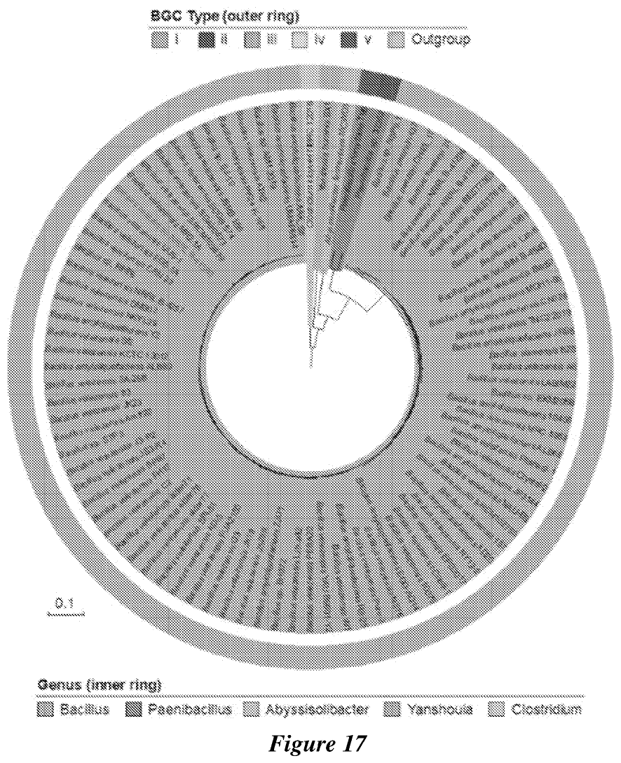

Diversity of phosphonoalanine Biosynthetic Pathways in Firmicutes. All phosphonoalanine encoding gene-neighborhoods of Firmicutes (Bacillota) in the NCBI non-redundant database were analyzed to understand the biosynthetic diversity and distribution of this pathway. 95 putative phosphonate/phosphinate biosynthetic gene clusters were identified where pepM and phosphonopyruvate-transminase are co-localized and ppd, pms, ppr are absent. The phosphonoalanine biosynthetic gene clusters were overwhelmingly encoded within Bacillus (95%), and significantly less abundant within Paenibacillus (3%), Abyssisolibacter (1%), and Yanshouia (1%) (FIG. 17; Table 14). Aside from the eight strains with only genus level identification, all remaining Bacillus strains (B. velezensis, B. amyloliquefasciens, B. swezeyi, B. subtilis, B. siamensis) belonged to the Bacillus subtilis species complex. While the majority of phosphonoalanine strains originated from soils (29%), plants (24%), and food (19%), isolates cultivated from freshwater (5%), marine (5%), animal (5%) and air (2%) sources also contained the pathway.

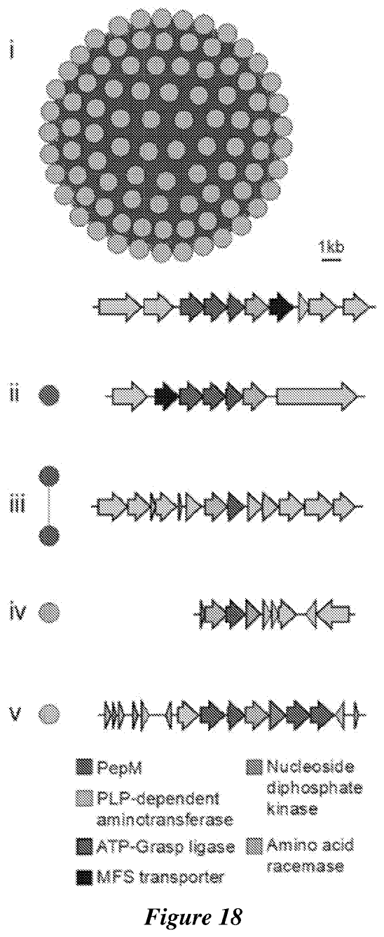

The phosphonoalanine biosynthetic gene clusters were classified into five gene-cluster families based on sequence similarity analysis of their phosphoenolpyruvate mutase proteins (FIG. 18). Group 1 contained all phosphonoalanine biosynthetic gene clusters from Bacillus. Their putative biosynthetic gene clusters were nearly identical to B. velezensis NRRL B-41580, indicating all are likely to produce phosphonopeptides with phosphonoalanine at the C-terminus. This was supported by the detection of phosphonoalanine, phosphonolamide E, and F from additional Bacillus strains within this gene-cluster family (FIG. 61).

Paenibacillus contained two different phosphonoalanine biosynthetic gene clusters. Like Bacillus and Streptomyces, the biosynthetic gene cluster of Paenibacillus forsythia T98 (Group 2) encodes putative ATP-Grasp ligases and an MFS transporter. However, low sequence similarity between these homologs hint at yet another series of phosphonoalamide-like compounds possibly composed of different amino acids. In contrast, the absence of phosphonate/phosphinate genes beyond pepM and putative phosphonopyruvate-transaminase in Paenibacillus sp. Strains 32352 and OAS669 (Group 4) suggest biosynthesis may terminate with phosphonoalanine. These are similar to the biosynthetic gene cluster from Yanshouia hominis BX1 (Group 4) apart from an amino acid racemase immediately downstream of pepM, which may invert the stereochemistry of phosphonoalanine. Lastly, Abyssisolibacter fermentans MCWD3 (Group 5) encodes several additional enzymes with the potential to ligate amino acids to phosphonoalanine. This may be representative of a pathway towards larger phosphonate/phosphinate tetrapeptides.

Considering the known limitations of strain isolation, it was speculated that the true diversity and abundance of phosphonoalanine biosynthetic gene clusters in Firmicutes is much higher. Even with the small number of pathways observed here, their distribution raises questions about their chemical ecology, evolution, and potential effects on resident microbiome communities. Nonetheless, the antimicrobial activities of 2 and 3 suggest other natural products containing phosphonoalanine may also be effective against bacteria associated with disease.

DISCUSSION The discovery of phosphonoalamide E and F from B. velezensis enriches the understanding of phosphonate biosynthesis and the chemical diversity of phosphonoalanine natural products. Although the biosynthetic gene clusters from Streptomyces and Bacillus both encode genes for putative phosphoenolpyruvate mutase, phosphonopyruvate-transaminase, and ATP-Grasp ligase enzymes, the strains yield markedly different phosphonoalanine phosphonopeptides. Streptomyces sp. B-2790 was previously shown to produce four phosphonate/phosphinate tripeptides with an N-terminal phosphonoalanine attached to combinations of alanine, valine, threonine, or isoleucine [A20]. In contrast, herein it was shown that alanyl di- and tripeptides with phosphonoalanine at the C-terminus are the products of B. velezensis. Given the conservation of biosynthetic genes between these two strains, the diversity of their products most likely stems from differential specificity of their encoded ATP-Grasp ligases. These enzymes catalyze peptide bond formation by first activating amino acids into an acylphosphate intermediate using ATP. This then serves as the substrate for nucleophilic attack by the amine group of the adjoining amino acid [A39]. Indeed, ATP-Grasp ligases are frequently observed within phosphonate/phosphinate biosynthetic gene clusters with functionally diverse activities. The biosynthetic gene clusters for the rhizocticin and plumbemycin phosphonopeptides encode ATP-Grasp ligases implicated in the attachment of amino acids onto the non-proteinogenic (Z)-_{L}-2-amino-5-phosphono-3-pentenoic acid (APPA) warhead [A19, A40]. ATP grasp-ligases in the valinophos biosynthetic gene cluster form a series of dipeptides that are attached to the terminal alcohol moiety of 2,3-dihydroxypropyl phosphonate via an unusual ester instead of a canonical amide bond [A23]. Finally, the role of these enzymes within argolaphos, pantophos, and O-phosphonoacetate serine biosynthesis remains elusive, as none are required for the biosynthesis of these phosphonate/phosphinate natural products despite their conservation within the biosynthetic gene clusters ┌A21, A22, A41┐. Understanding the catalytic diversity of the ATP-Grasp ligases and the molecular determinants of their activities will be required to improve bioinformatic predictions of end-products during phosphonate/phosphinate genome mining.

The compositional differences between phosphonoalamide F (_{L}-alanine-_{L}-alanine-_{L}-phosphonoalanine; from Bacillus) and phosphonoalamide A (_{L}-phosphonoalanine-_{L}-alanine-_{L}-valine, from Streptomyces) resulted in different inhibitory activities against the same panel of bacteria. Phosphonoalamide F exhibited greater inhibitory activity (i.e. lower MICs) against E. coli K12, S. enterica LT2, P. aeuroginosa K and S. marcescens B-2544 than was previously measured for phosphonoalamide A. This may reflect the natural specificities of different oligopeptide transporters in recognizing various phosphonopeptides and importing them across bacterial cell walls. Indeed, the “Trojan horse” mechanism underlies the antimicrobial activities of many phosphonopeptides, including bialaphos, dehydrophos, and rhizocticin [A42-A44]. Activity is dependent upon natural uptake and subsequent hydrolysis by cytosolic peptidases, releasing the active phosphonate “warhead”. This is best exemplified by rhizocticin, plumbemycin, and phosacetymicin, which display differential efficacy against bacteria and fungi depending on the composition of amino acids ligated to APPA [A19, A43, A45]. The activity profiles of the phosphonoalamides provides further evidence that phosphonopeptide potency and selectivity may be tunable via chemical modifications designed to facilitate cellular import.

Beyond pharmaceutical development, the Bacillus-derived phosphonoalamides have the potential to address the significant need for commercial bactericides in agriculture. These phosphonoalamides inhibited E. rhapontici and P. ananatis, both of which are major pests of numerous cash crops. E. rhapontici is the causative agent of bacterial soft rot in multiple vegetables including rhubarb, sugar beets and tomato, as well as pink seed disease of many commercial grains [A46]. P. ananatis is a pathogen of maize, rice, tomato, and melons, but is most burdensome in onion agriculture where entire harvests can be lost to it [A47, A48]. The compounds also inhibited S. marcescens and P. larvae, devastating bacterial pathogens of the Western honeybee, an essential pollinator used in 90% of commercial agriculture worldwide [A49]. S. marcescens is a natural member of the honeybee gut microbiome, but also an opportunistic pathogen which may contribute to global population decline [A50]. In contrast, P. larvae is the causative agent of American Foulbrood, a fatal honeybee disease that can lead to widespread outbreaks disrupting natural and commercial pollination. The bacterium invades and cannibalizes honeybee larvae, releasing spores that are spread from hive to hive [A51]. Given the need for effective treatments to mitigate losses from these diseases, the phosphonoalamides represent a potential opportunity for development as commercial pesticides. Understanding the molecular target of the phosphonoalamides and potential mechanisms of resistance will inform their suitability for commercial use and the creation of more potent analogs.

Finally, these results highlight the opportunity for phosphonate/phosphinate natural product discovery in taxa outside actinobacteria. Although actinobacteria harbor the greatest diversity in their natural product pathways and have a storied history of producing pharmaceutically and agriculturally useful compounds, phosphonate/phosphinate biosynthetic gene clusters are abundant within other taxa, including the Firmicutes and Proteobacteria [A16]. Thus, the potential utility and physiological importance of phosphonates produced by these microbes should not be overlooked.

### Materials and Methods

Strains, Media, and General Culture Conditions. B. velezensis NRRL B-41580, B. velezensis NRRL B-4257, B. swezeyi NRRL B-41282, and B. swezeyi NRRL B-41294 were obtained from the USDA NRRL strain collection (Peoria, IL). The full list of bioassay strains used in this study are listed in Table 12. E. coli, S. enterica, and M. smegmatis, were grown at 37° C. All other strains were grown at 30° C.

All chemicals and reagents were from Sigma-Aldrich, Fisher Scientific, or VWR. Media used in this study included nutrient broth (NB: 3 g beef extract, 5 g peptone), GUBC [A18], R2AS [A18], tryptic soy broth (TSB; 17 g tryptone, 3 g soytone, 2.5 g dextrose, 5 g NaCl, 2.5 g K₂HPO₄; pH to 7.3 prior to autoclaving), rhizocticin media [A43], M9 with 20 mM glucose, YPD (10 g yeast extract, 20 g peptone, 20 g dextrose), yeast minimal media (YMM; 6.7 g yeast nitrogen base without amino acids, 20 g dextrose), malt extract media (MEM; 20 g malt extract, 20 g dextrose, 6 g peptone). M9 glucose medium was supplemented with 1 mM thiamine-HCl and 0.05 mM nicotinamide for Staphylococcus (SSM9PR medium) and solution B metals for Paenibacillus larvae [A18]. All components were dissolved in deionized water (dIH₂O). 16 g of agar was added per liter of media.

Bioinformatics. The genomic assembly for Mycobacteroides abscessus subsp. Massilience strain aerosol_aerosol_3 (GCA_900138605.1) was retrieved from NCBI and analyzed locally. The taxonomic origin of contigs was determined by analyzing the first 2-kb of each against the NCBI non-redundant nucleotide database using BLASTn and recording the genus of the top hit. Complete genomes of Mycobacterium, Mycobacteroides, and Bacillus velezensis within the NCBI Genomes database were retrieved and analyzed locally.

Phosphoenolpyruvate mutase sequences from actinobacteria were identified and retrieved from NCBI as previously described [A20]. The phosphoenolpyruvate mutase (WP_239689795) and putative phosphonopyruvate-transaminase (WP_053285252) from B. velezensis NRRL B-41580 were used as query sequences for cblaster [A52] to identify putative gene neighborhoods within Firmicutes. Genbank files were downloaded from NCBI using the rentrez R package and manually analyzed to verify the absence of phosphonopyruvate decarboxylase, phosphonopyruvate reductase, and phosphonomethylmalate synthase. Open reading frames and synteny were analyzed using cblaster, clinker [A53], or EasyFig [A54]. BLAST analyses were performed against the NCBI non-redundant (nr) protein database and Pfam [A55]. Sequence similarity networks were created using the Enzyme Function Initiative enzyme similarity tool [A56] and visualized using Cytoscape.

16S rRNA gene sequences were identified from assemblies of phosphonoalanine-biosynthetic gene cluster containing Firmicutes (Table 14). Wherever possible, partial 16S sequences within assemblies were combined with overlapping fragments to create more complete sequences. The 16S rRNA gene sequence from Clostridium kluyveri NBRC 12016 served as the outgroup. Sequences were aligned using the MAFFT [A57] and phylogenic tree calculated using FastTree [A58].

TABLE 14
Firmicutes with genes for phosphonoalanine biosynthesis

| Strain | Assembly | Source | Group |
|---|---|---|---|
| Abyssisolibacter fermentans MCWD3 | GCF_001559865.1 | Marine | 4 |
| Bacillus amyloliquefaciens 10456 | GCF_009933355.1 | Marine | 1 |
| Bacillus amyloliquefaciens AAK_S6 | GCF_011440355.1 | Soil | 1 |
| Bacillus amyloliquefaciens AD20 | GCF_018791865.1 | Freshwater | 1 |
| Bacillus amyloliquefaciens ALB65 | GCF_003149715.1 | Plant | 1 |
| Bacillus amyloliquefaciens ALB69 | GCF_003149695.1 | Plant | 1 |
| Bacillus amyloliquefaciens EGD-AQ14 | GCF_000465655.1 | Soil | 1 |
| Bacillus amyloliquefaciens JP3144 | GCF_019880425.1 | Soil | 1 |
| Bacillus amyloliquefaciens JRS5 | GCF_001286945.1 | Soil | 1 |
| Bacillus amyloliquefaciens MOH1-5b | GCF_014792065.1 | Plant | 1 |
| Bacillus amyloliquefaciens R8-25 | GCF_013341255.1 | Plant | 1 |
| Bacillus amyloliquefaciens UMAF6614 | GCF_001593785.1 | Plant | 1 |
| Bacillus amyloliquefaciens Y2 | GCF_000262385.1 | Soil | 1 |
| Bacillus amyloliquefaciens ZJU1 | GCF_007362635.1 | Plant | 1 |
| Bacillus siamensis B28 | GCF_016313165.1 | Food-derived | 1 |
| Bacillus sp. AM1 2019 | GCF_009906915.1 | Unknown | 1 |
| Bacillus sp. BH072 | GCF_000827045.1 | Food-derived | 1 |
| Bacillus sp. EKM208B | GCF_011800725.1 | Plant | 1 |
| Bacillus sp. Lzh-5 | GCF_002763675.1 | Soil | 1 |
| Bacillus sp. NSP9.1 | GCF_000465465.2 | Food-derived | 1 |
| Bacillus sp. RHF6 | GCF_016757495.1 | Soil | 1 |
| Bacillus sp. SJ-10 | GCF_002843505.1 | Food-derived | 1 |
| Bacillus sp. STP3 | GCF_003946965.1 | Soil | 1 |
| Bacillus subtilis BEST7003 | GCF_000523045.1 | Lab-derived | 1 |
| Bacillus subtilis BEST7613 | GCF_000328745.1 | Lab-derived | 1 |
| Bacillus swezeyi 21 | GCF_008682385.1 | Unknown | 1 |
| Bacillus swezeyi 427 | GCF_008682325.1 | Unknown | 1 |
| Bacillus swezeyi CH43_1T | GCF_008180465.1 | Freshwater | 1 |
| Bacillus swezeyi NRRL B-41282 | GCF_001969555.1 | Soil | 1 |
| Bacillus swezeyi NRRL B-41294 | GCF_001969815.1 | Soil | 1 |
| Bacillus velezensis 10075 | GCF_002893845.1 | Food-derived | 1 |
| Bacillus velezensis 157 | GCF_002216755.1 | Plant | 1 |
| Bacillus velezensis 27-5 | GCF_009935295.1 | Soil | 1 |
| Bacillus velezensis 3A-25B | GCF_002269705.1 | Soil | 1 |
| Bacillus velezensis 83 | GCF_004101805.1 | Plant | 1 |
| Bacillus velezensis A6 | GCF_004118065.1 | Soil | 1 |
| Bacillus velezensis AD-3 | GCF_017347625.1 | Animal | 1 |
| Bacillus velezensis alder71 | GCF_020179855.1 | Plant | 1 |
| Bacillus velezensis alder76 | GCF_020177005.1 | Plant | 1 |
| Bacillus velezensis alder77 | GCF_020179885.1 | Plant | 1 |
| Bacillus velezensis Amfr20 | GCF_016772005.1 | Plant | 1 |
| Bacillus velezensis ATR2 | GCF_002761535.1 | Soil | 1 |
| Bacillus velezensis Bac57 | GCF_003667745.1 | Marine | 1 |
| Bacillus velezensis BIM B-454D | GCF_019880485.1 | Soil | 1 |
| Bacillus velezensis C2 | GCF_002245385.1 | Plant | 1 |
| Bacillus velezensis CN026 | GCF_002796465.1 | Animal | 1 |
| Bacillus velezensis Cob9-1 | GCF_907164745.1 | Food-derived | 1 |
| Bacillus velezensis CRN 23 | GCF_013266865.1 | Animal | 1 |
| Bacillus velezensis Crystal-2 | GCF_907164755.1 | Food-derived | 1 |
| Bacillus velezensis CS1.10S | GCF_003665175.1 | Food-derived | 1 |
| Bacillus velezensis DMB07 | GCF_020150015.1 | Food-derived | 1 |
| Bacillus velezensis EG5.1A | GCF_013285045.1 | Plant | 1 |
| Bacillus velezensis EPS2017 | GCF_000786145.2 | Plant | 1 |
| Bacillus velezensis FH17 | GCF_003990425.1 | Soil | 1 |
| Bacillus velezensis FUA2155 | GCF_007844975.1 | Food-derived | 1 |
| Bacillus velezensis G3 | GCF_009768905.1 | Unknown | 1 |
| Bacillus velezensis GB1 | GCF_002165455.1 | Plant | 1 |
| Bacillus velezensis J3-P2 | GCF_019749155.1 | Air | 1 |
| Bacillus velezensis JK19 | GCF_018278845.1 | Soil | 1 |
| Bacillus velezensis JK23 | GCF_018224265.1 | Soil | 1 |
| Bacillus velezensis JS25R | GCF_000769555.1 | Soil | 1 |
| Bacillus velezensis JS3-R4 | GCF_019749135.1 | Air | 1 |
| Bacillus velezensis JSRB 166 | GCF_014048285.1 | Food-derived | 1 |
| Bacillus velezensis JW | GCF_002909615.1 | Animal | 1 |
| Bacillus velezensis KCTC 13012 | GCF_001267695.1 | Freshwater | 1 |
| Bacillus velezensis LABIM22 | GCF_013401395.2 | Soil | 1 |
| Bacillus velezensis Lzh-a42 | GCF_002844125.1 | Soil | 1 |
| Bacillus velezensis MR2.1A | GCF_013285105.1 | Plant | 1 |
| Bacillus velezensis MRC 5958 | GCF_002943735.1 | Freshwater | 1 |
| Bacillus velezensis NAU-B3 | GCF_000493375.1 | Unknown | 1 |
| Bacillus velezensis NKYL29 | GCF_000740715.1 | Soil | 1 |
| Bacillus velezensis NRRL B-41580 | GCF_001461825.1 | Freshwater | 1 |
| Bacillus velezensis NRRL B-4257 | GCF_001461845.1 | Soil | 1 |
| Bacillus velezensis NST6 | GCF_014180705.2 | Soil | 1 |
| Bacillus velezensis NY12-2 | GCF_003721475.1 | Food-derived | 1 |
| Bacillus velezensis PEBA20 | GCF_016859395.1 | Plant | 1 |
| Bacillus velezensis Pilsner2-1 | GCF_907164765.1 | Food-derived | 1 |
| Bacillus velezensis PK24 K-165 | GCF_018729415.1 | Plant | 1 |
| Bacillus velezensis Pure-2 | GCF_907164835.1 | Food-derived | 1 |
| Bacillus velezensis S3 | GCF_016901175.1 | Plant | 1 |
| Bacillus velezensis SCGB 574 | GCF_002327165.1 | Food-derived | 1 |
| Bacillus velezensis Sf2 | GCF_016772025.1 | Plant | 1 |
| Bacillus velezensis SGAir0473 | GCF_003184945.1 | Air | 1 |
| Bacillus velezensis SPB7 | GCF_005518225.1 | Marine | 1 |
| Bacillus velezensis SPL51 | GCF_012935315.1 | Plant | 1 |
| Bacillus velezensis SRCM102755 | GCF_009913295.1 | Food-derived | 1 |
| Bacillus velezensis SRCM103639 | GCF_004115935.1 | Food-derived | 1 |
| Bacillus velezensis TNC2 2019 | GCF_010614765.1 | Food-derived | 1 |
| Bacillus velezensis UUS-1 | GCF_004378445.1 | Marine | 1 |
| Bacillus velezensis W1 | GCF_003265265.1 | Unknown | 1 |
| Bacillus velezensis YAU B9601-Y2 | GCF_000284395.1 | Soil | 1 |
| Paenibacillus forsythiae T98 | GCF_000520735.1 | Soil | 5 |
| Paenibacillus sp. 32352 | GCF_002042965.1 | Soil | 2 |
| Paenibacillus sp. OAS669 | GCF_014873645.1 | Soil | 2 |
| Yanshouia hominis BX1 | GCF_014385025.1 | Animal | 3 |

Production Screening. B. velezensis NRRL B-4257, B. velezensis NRRL B-41580, B. swezeyi NRRL B-41282, and B. swezeyi NRRL B-41294 were revived onto TSB plates from glycerol stocks and inoculated in 20×150 mm test tubes containing 5 mL of the same medium. Starter cultures were grown at 30° C. for 24 hours on a rotary shaker (220 rpm) and used to inoculate 125 mL baffled flasks containing 25 mL of NB, GUBC, R2AS, TSB, or RM (500 μL per flask). These were incubated on a rotary shaker (30° C., 220 rpm) for 3 days. Timecourse experiments were performed in 2.5 L Ultra-Yield flasks containing 1 L of production media. These were inoculated with 10 mL of starter cultures and incubated on a rotary shaker (30° C., 220 rpm). Samples were withdrawn (25 mL) at 3, 5, and 7 days of growth.

Cultures were harvested by centrifugation at 10,000 RPM, 4° C., for 10 minutes. Clarified supernatants were lyophilized to dryness and reconstituted in 1 mL of dI H₂O. Concentrated extracts were then amended with 10% D₂O for ³¹P NMR analysis. Putative phosphonic acids were identified as ³¹P NMR resonances with chemical shifts 8 ppm or greater.

Production Scale-Up. A starter culture of B. velezensis NRRL B-41580 (as above) was used to inoculate 500 mL Fernbach flasks containing 125 mL TSB medium. After 36 hours of growth at 30° C. on a rotary shaker (220 RPM), these seed cultures were used to inoculate 13 individual 2.5 L Ultra-Yield flasks each containing 1 L of TSB. These production cultures were grown for 7 days at 30° C. on a rotary shaker (220 RPM). Cultures were harvested by centrifugation (10,000 RPM, 4° C., for 10 minutes). Clarified supernatant was removed and set aside. Cell pellets were resuspended in 500 mL methanol and vigorously agitated by vortexing to extract residual metabolites. The aqueous and methanolic fractions combined to yield 13.5 L of starting material.

NMR Analyses and Mass Spectrometry. NMR experiments were performed at the Ohio State University Campus Chemical Instrument Center. All NMR spectra were acquired at 25° C. on a Bruker Avance III HD Ascend 600 MHz spectrometer (600 MHz for H, 150 MHZ for ¹³C, and 243 MHz for 31P) equipped with a Bruker 5 mm Smart Broadband Observe solution probe (BBFO), a Bruker Avance Neo 400 MHz spectrometer (400 MHz for ¹H, 100 MHz for ¹³C and 162 MHz for ³¹P) equipped with a 5 mm Prodigy Cryoprobe, or a Bruker Avance III HD Ascend 700 MHz spectrometer (700 MHz for ¹H, 176 MHz for ¹³C, and 283 MHz for ³¹P) equipped with a 5 mm Triple-resonance Observe (TXO) cryoprobe. Spectra were processed using MestReNova 12 software. HRMS and HRMS/MS were performed on an Agilent 6540 UHD Accurate-Mass Q-TOF system equipped with an Agilent 1260 Infinity II HPLC as previously described [A20].

Marfey's Analysis. Compounds 2 and 3 (0.2 mg) were dissolved in 0.5 mL of 6N HCl and heated to 100° C. in a sealed reaction vial for 16 hours. Samples were dried at 40° C. under a gentle stream of air to remove HCl. Hydrolysates were dissolved in 50 μL of water and transferred to microcentrifuge tubes, and 50 mM solutions (50 μL) of each amino acid standard were prepared. Each sample or standard was combined with 20 μL of 1M NaHCO₃ and 100 μL of a 1% solution of FDAA in acetone and incubated in a heating block at 40° C. for 1 hour. Samples were cooled to room temperature and neutralized with 20 μL of 1M HCl. Derivatized amino acid standards and hydrolysates were diluted 50-fold into 10% MeCN with 0.1% formic acid. Derivatized phosphonoalanine solutions were diluted 50-fold into water with 0.1% formic acid. These were analyzed by LC-MS using Phenomenex Fusion-RP column (2×100 mm, 4 μm) using a gradient of 0-100% MeCN with 0.1% formic acid over 30 min.

Antimicrobial Assays. Compounds 2 and 3 were repeatedly lyophilized and exchanged with ultra-pure dI H₂O to ensure all residual modifier was removed prior to assays. Susceptibility testing was performed using the microbroth dilution method in 96 round-well microtiter plates as previously described [A20]. Compounds were prepared as 50× of stocks (10 Mm) by dissolving in Di H2O. All bacterial strains were grown and assayed M9 glucose media (with amendments added as required), and yeast strains in YMM. Controls wells contained 200 UM kanamycin (for bacteria) or nystatin (for yeasts), no compound addition (vehicle only), and no cell addition (added media instead). Culture densities (OD600) were recorded using a Bio-Rad xMark microplate spectrophotometer after 16 hours for all strains except bacilli, which were recorded after 24 hours. Minimum inhibitory concentration (MIC) was defined as the lowest concentration of compound that resulted in ≥90% growth inhibition. MICs values are from triplicate assays performed on separate days. Disk diffusion assays were used to assess antimicrobial activity of the purified compounds against filamentous fungi and against Mycobacterium smegmatis [A20].

### Structure Elucidation

### Chemical Data of Purified Compounds

2-amino-3-phosphonopropanoic acid (1; phosphonoalanine). White powder; ¹H NMR (400 MHZ, D₂O, δ, ppm, J/Hz), 3.79 (¹H, m, H-2), 2.18 (¹H, m, H-1), 1.87 (¹H, tdt, J=14.5, 11.3, 0.7 Hz, H-1); ³¹P NMR (162 MHZ, D₂O, δ, ppm), 18.71 (P—CH₂, s, P-a); ESI HRMS calcd for C₃H₉NO₅P⁺: 170.0213. obs: 170.1212 (Δppm=−0.4).

(2-amino-3-phosphonopropanoyl) alanine (2: Phosphonoalamide E). White powder; ¹H NMR (700 MHZ, 90% H₂O/10% D₂O, δ, ppm, J/Hz), 8.27 (NH, d, J=6.9 Hz, N-b), 4.33 (1H, m, H-2), 4.03 (1H, q, J=7.1 Hz, H-2′), 2.10 (1H, m, H-1), 1.92 (1H, td, J=15.2, 10.7 Hz, H-1), 1.46 (3H, d, J=7.18 Hz, H-3′); ¹³C NMR (176 MHZ, 100% D₂O, δ, ppm, J/Hz), 177.18 (qC, d, J=14.85 Hz, C-3), 170.20 (qC, s, C-1′), 51.14 (CH, d, J=5.27 Hz, C-2), 49.34 (CH, s, C-2′), 29.71 (CH₂, d, J=133.1, C-1), 16.25 (CH₃, s, C-3′); ³¹P NMR (162 MHZ, D₂O, δ, ppm), 19.54 (P—CH₂, dt, =18.0, 14.6 Hz, P-a); ESI HRMS calcd for C₆H₁₄N₂O₆P⁺: 241.0584. obs: 241.0583 (Δppm=−0.4).

(2-amino-3-phosphonopropanoyl) alanylalanine (3; Phosphonoalamide F). White powder; ¹H NMR (700 MHZ, 90% H₂O/10% D₂O, δ, ppm, J/Hz), 8.55 (NH, d, J=6.0 Hz, N-c), 8.23 (NH, d, J=6.5 Hz, N-b), 4.37 (CH, m, H-2), 4.29 (CH, p, J=7.1 Hz, H-2′), 4.00 (CH, q, J=7.1, H-2″), 2.09 (1H, ddd, J=5.0, 15.3, 17.6, H-1), 1.98 (1H, td, J=8.9, 15.6, 15.4 Hz, H-1), 1.46 (3H, d, J=7.1 Hz, H-3″), 1.33 (3H, d, J=7.2 Hz, H-3′); ¹³C NMR (176 MHz, 100% D₂O, δ, ppm, J/Hz), 175.73 (qC, d, J=12.5 Hz, C-3), 174.17 (qC, s, C-1′), 170.69 (qC, s, C-1″), 50.10 (CH, d, J=4.6 Hz, C-2), 49.82 (CH, s, C-2′), 49.15 (CH, s, C-2″), 16.56 (CH₃, s, C-3″), 16.30 (CH₃, s, C-3′); ³¹P NMR (162 MHZ, D₂O, δ, ppm), 19.13 (P—CH₂, q, J_{CP}=16.4 Hz, P-a); ESI HRMS calcd for C₉H₁₉N₃O₇P⁺: 312.0955. obs: 312.0952 (Δppm=−0.9).

### REFERENCES

A1. Jomaa H et al. 1999. Inhibitors of the nonmevalonate pathway of isoprenoid biosynthesis as antimalarial drugs. Science 285:1573-6.
A2. Kuroda Y et al. 1980. Studies on new phosphonic acid antibiotics. IV. Structure determination of FR-33289, FR-31564 and FR-32863. J Antibiot (Tokyo) 33:29-35.
A3. Okuhara M et al. 1980. Studies on new phosphonic acid antibiotics. III. Isolation and characterization of FR-31564, FR-32863 and FR-33289. J Antibiot (Tokyo) 33:24-8.
A4. Okuhara M et al. 1980. Studies on new phosphonic acid antibiotics. I. FR-900098, isolation and characterization. J Antibiot (Tokyo) 33:13-7.
A5. Wiesner J et al. 2016. FR-900098, an antimalarial development candidate that inhibits the non-mevalonate isoprenoid biosynthesis pathway, shows no evidence of acute toxicity and genotoxicity. Virulence 7:718-28.
A6. Missinou M A et al. 2002. Fosmidomycin for malaria. Lancet 360:1941-2.
A7. Hendlin D et al. 1969. Phosphonomycin, a new antibiotic produced by strains of streptomyces. Science 166:122-3.
A8. Shoji J et al. 1986. Production of fosfomycin (phosphonomycin) by Pseudomonas syringae. J Antibiot (Tokyo) 39:1011-2.
A9. Silver L L. 2017. Fosfomycin: Mechanism and Resistance. Cold Spring Harb Perspect Med 7.
A10. Takano H K et al. 2020. Glufosinate-ammonium: a review of the current state of knowledge. Pest Manag Sci 76:3911-3925.
A11. Ju K S et al. 2022. Convergent and divergent biosynthetic strategies towards phosphonic acid natural products. Curr Opin Chem Biol 71:102214.
A12. Horsman G P et al. 2017. Phosphonate Biochemistry. Chem Rev 117:5704-5783.
A13. Seidel H M et al. 1988. Phosphonate biosynthesis: isolation of the enzyme responsible for the formation of a carbon-phosphorus bond. Nature 335:457-8.
A14. Bowman E et al. 1988. Catalysis and thermodynamics of the phosphoenolpyruvate/phosphonopyruvate rearrangement. Entry into the phosphonate class of naturally occurring organophosphorus compounds. J Am Chem Soc 110:5575-5576.
A15. Hidaka T et al. 1989. Studies on the biosynthesis of bialaphos (SF-1293). 9. Biochemical mechanism of C—P bond formation in bialaphos: discovery of phosphoenolpyruvate phosphomutase which catalyzes the formation of phosphonopyruvate from phosphoenolpyruvate. J Antibiot (Tokyo) 42:491-4.
A16. Yu X et al. 2013. Diversity and abundance of phosphonate biosynthetic genes in nature. Proc Natl Acad Sci USA 110:20759-64.
A17. Ju K S et al. 2014. Genomics-enabled discovery of phosphonate natural products and their biosynthetic pathways. J Ind Microbiol Biotechnol 41:345-56.
A18. Ju K S et al. 2015. Discovery of phosphonic acid natural products by mining the genomes of 10,000 actinomycetes. Proc Natl Acad Sci USA 112:12175-80.
A19. Evans B S et al. 2013. Discovery of the antibiotic phosacetamycin via a new mass spectrometry-based method for phosphonic acid detection. ACS Chem Biol 8:908-13.
A20. Kayrouz C M et al. 2020. Genome Mining Reveals the Phosphonoalamide Natural Products and a New Route in Phosphonic Acid Biosynthesis. ACS Chem Biol 15:1921-1929.
A21. Polidore A L A et al. 2021. A Phosphonate Natural Product Made by Pantoea ananatis is Necessary and Sufficient for the Hallmark Lesions of Onion Center Rot. mBio 12:1-18.
A22. Zhang Y et al. 2022. Biosynthesis of Argolaphos Illuminates the Unusual Biochemical Origins of Aminomethylphosphonate and Na-Hydroxyarginine Containing Natural Products. J Am Chem Soc 144:9634-9644.
A23. Zhang Y et al. 2022. Valinophos Reveals a New Route in Microbial Phosphonate Biosynthesis That Is Broadly Conserved in Nature. J Am Chem Soc 144:9938-9948.
A24. Horiguchi M. 1984. Occurrence, identification, and properties of phosphonic acid and phosphinic acids, p 24-52. In Hori T, Horiguchi M, Hayashi A (ed), Biochemistry of Natural C—P Compounds. Japanese Association for Research on the Biochemistry of C—P Compounds, Shiga, Japan.
A25. Parkinson E I et al. 2019. Fosmidomycin biosynthesis diverges from related phosphonate natural products. Nat Chem Biol 15:1049-1056.
A26. O'Connor J J et al. 1994. Long-lasting enhancement of NMDA receptor-mediated synaptic transmission by metabotropic glutamate receptor activation. Nature 367:557-9.
A27. Hildebrand R L. 1983. The Role of Phosphonates in Living Systems. CRC Press, Boca Raton.
A28. Kulakova A N et al. 2009. Expression of the phosphonoalanine-degradative gene cluster from Variovorax sp. Pal2 is induced by growth on phosphonoalanine and phosphonopyruvate. FEMS Microbiol Lett 292:100-6.
A29. Kulakova A N et al. 2003. The purification and characterization of phosphonopyruvate hydrolase, a novel carbon-phosphorus bond cleavage enzyme from Variovorax sp Pal2. J Biol Chem 278:23426-31.
A30. Zhang G et al. 2003. The phosphonopyruvate decarboxylase from Bacteroides fragilis. J Biol Chem 278:41302-8.
A31. Nakashita H et al. 1997. Studies on the biosynthesis of bialaphos. Biochemical mechanism of C—P bond formation: discovery of phosphonopyruvate decarboxylase which catalyzes the formation of phosphonoacetaldehyde from phosphonopyruvate. J Antibiot (Tokyo) 50:212-9.
A32. Eliot A C et al. 2008. Cloning, expression, and biochemical characterization of Streptomyces rubellomurinus genes required for biosynthesis of antimalarial compound FR900098. Chem Biol 15:765-70.
A33. Newton G L et al. 2008. Biosynthesis and functions of mycothiol, the unique protective thiol of Actinobacteria. Microbiol Mol Biol Rev 72:471-94.
A34. Shyam M et al. 2021. The Mycobactin Biosynthesis Pathway: A Prospective Therapeutic Target in the Battle against Tuberculosis. J Med Chem 64:71-100.
A35. Ripoll F et al. 2009. Non mycobacterial virulence genes in the genome of the emerging pathogen Mycobacterium abscessus. PLOS One 4: e5660.
A36. Choo S W et al. 2020. Comparative genome analyses of Mycobacteroides immunogenum reveals two potential novel subspecies. Microb Genom 6.
A37. Kayrouz C M et al. 2020. Genome mining reveals the phosphonoalamide natural products and a new route in phosphonic acid biosynthesis. ACS Chemical Biology 15:1921-1929.
A38. Rabbee M F et al. 2019. Bacillus velezensis: A Valuable Member of Bioactive Molecules within Plant Microbiomes. Molecules 24.
A39. Fawaz M V et al. 2011. The ATP-grasp enzymes. Bioorg Chem 39:185-91.
A40. Borisova S A et al. 2010. Biosynthesis of rhizocticins, antifungal phosphonate oligopeptides produced by Bacillus subtilis ATCC6633. Chem Biol 17:28-37.
A41. Freestone T S et al. 2017. Discovery of a Phosphonoacetic Acid Derived Natural Product by Pathway Refactoring. ACS Synth Biol 6:217-223.
A42. Circello B T et al. 2011. The antibiotic dehydrophos is converted to a toxic pyruvate analog by peptide bond cleavage in Salmonella enterica. Antimicrob Agents Chemother 55:3357-62.
A43. Kugler M et al. 1990. Rhizocticin A, an antifungal phosphono-oligopeptide of Bacillus subtilis ATCC 6633: biological properties. Arch Microbiol 153:276-81.
A44. Abouhamad W N et al. 1991. Peptide transport and chemotaxis in Escherichia coli and Salmonella typhimurium: characterization of the dipeptide permease (Dpp) and the dipeptide-binding protein. Mol Microbiol 5:1035-47.
A45. Park B K et al. 1977. Studies on new antimetabolite N-1409. Agric Biol Chem 41:161-167.
A46. Huang H C et al. 2003. Biology and epidemiology of Erwinia rhapontici, caual agent of pink seend and crown rot of plants. Plant Pathol Bull 12:69-76.
A47. Coutinho T A et al. 2009. Pantoea ananatis: an unconventional plant pathogen. Mol Plant Pathol 10:325-35.
A48. Gitaitis R D et al. 1997. First Report of a Leaf Blight, Seed Stalk Rot, and Bulb Decay of Onion by Pantoea ananas in Georgia. Plant Dis 81:1096.
A49. Funfhaus A et al. 2018. Bacterial pathogens of bees. Curr Opin Insect Sci 26:89-96.
A50. Raymann K et al. 2018. Pathogenicity of Serratia marcescens Strains in Honey Bees. mBio 9.
A51. Poppinga L et al. 2015. Molecular pathogenesis of American Foulbrood: how Paenibacillus larvae kills honey bee larvae. Curr Opin Insect Sci 10:29-36.
A52. Gilchrist C L M et al. 2021. cblaster: a remote search tool for rapid identification and visualization of homologous gene clusters. Bioinform Adv 1: vbab016.
A53. Gilchrist C L M et al. 2021. Clinker & clustermap.js: Automatic generation of gene cluster comparison figures. Bioinformatics doi: 10.1093/bioinformatics/btab007.
A54. Sullivan M J et al. 2011. Easyfig: a genome comparison visualizer. Bioinformatics 27:1009-10.
A55. Mistry J et al. 2021. Pfam: The protein families database in 2021. Nucleic Acids Res 49: D412-D419.
A56. Gerlt J A et al. 2015. Enzyme Function Initiative-Enzyme Similarity Tool (EFI-EST): A web tool for generating protein sequence similarity networks. Biochim Biophys Acta 1854:1019-37.
A57. Katoh K et al. 2019. MAFFT online service: multiple sequence alignment, interactive sequence choice and visualization. Brief Bioinform 20:1160-1166.
A58. Price M N et al. 2010. FastTree 2—approximately maximum-likelihood trees for large alignments. PLOS One 5:e9490.

### Example 3—Phosphonopeptide Inhibitors of Plant and Animal Pathogens

Described herein is a means to produce and obtain a specific series of phosphonopeptide compounds from a microbial strain and their application as antimicrobial agents against plant and animal pathogens is demonstrated. The compounds inhibit bacterial strains of concern to human health and thus are potential anti-infective agents. Additionally, the compounds demonstrate activity against pathogens of agricultural concern and thus are potential pesticide agents. Notably, potent inhibition of the bacterial pathogen of onions was observed, which results in an estimated $60 million loss per annum in the USA and for which there are no bactericides on market. Thus, the compounds have utility for both pharmaceutical and agricultural applications.

### Example 4

Phosphonate natural products are renowned for inhibitory activities which underly their development as antibiotics and pesticides. Although most phosphonate natural products have been isolated from Actinobacteria, bioinformatic surveys suggest many other bacterial phyla are replete with phosphonate biosynthetic potential. While mining actinobacterial genomes, a contaminated Mycobacteroides dataset was encountered which encoded a biosynthetic gene cluster predicted to produce novel phosphonate compounds. Sequence deconvolution revealed that the contig containing this cluster, as well as many others, belonged to a contaminating Bacillus, and are broadly conserved among multiple species, including the epiphyte B. velezensis. Isolation and structure elucidation revealed a new di- and tripeptide composed of L-alanine and a C-terminal L-phosphonoalanine which herein are named phosphonoalamide E and F. These compounds display broad-spectrum antibacterial activity, with strong inhibition against the agricultural pests responsible for vegetable soft rot (Erwinia rhapontici), onion rot (Pantoea ananatis), and American foulbrood (Paenibacillus larvae).

In non-resistant strains of the honeybee pathogen P. larvae, antibiotics can prevent the vegetative state of the bacterium forming. Prevention of germination of American foulbrood spores is possible using oxytetracycline hydrochloride (Terramycin). Another drug treatment, tylosin tartrate, was approved by the US Food and Drug Administration (FDA) in 2005.

Chemical treatment is sometimes used prophylactically, but certain strains of the bacterium seem to be rapidly developing resistance. Once the prophylactic treatment is suspended, the American foulbrood spores germinate, leading to a disease outbreak.

Alternative treatments are currently under investigation. One example is phage therapy. Another promising approach might be the use of lactic acid-producing bacteria as a treatment for AFB.

In January 2023, the United States Department of Agriculture approved the world's first vaccine for bees. The vaccine protects the bees from foulbrood and is dispensed by adding an inactive version of the bacteria to royal jelly consumed by worker bees, who feed the queen bee, who in turn passes immunity to her offspring.

Phosphonate natural products produced by bacteria have been a source of clinical antibiotics and commercial pesticides.

Herein, two new phosphonopeptides produced by B. velezensis with antibacterial activity against human and plant pathogens, including those responsible for widespread soft rot in crops and American foulbrood, are described. The results shed new insight on the natural chemical diversity phosphonates and suggest these compounds could be developed as effective antibiotics for use in medicine and or agriculture.

Disclosed herein are compositions comprising a compound of the formula below or a salt or derivative thereof, wherein the compound is referred to herein as phosphonoalamide E:

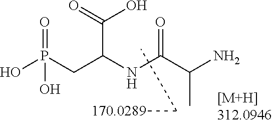

Also provided are compositions comprising a compound of the formula below or a salt or derivative thereof, wherein the compound is referred to herein as phosphonoalamide F:

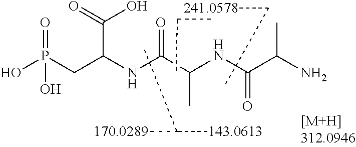

Also disclosed herein are nucleic acids encoding the composition herein, a vector encoding said nucleic acid, and a cell comprising said vector.

Also provided are methods of purifying a phosphonate produced by a cell, the method comprising providing a combination of two or more of the elements shown in FIG. 4 and/or FIG. 13. In some examples, the phosphonate is a phosphonoalanine, phosphonoalamide E, and/or phosphonoalamide F.

Also provided are methods of killing bacteria, the method comprising exposing bacteria to phosphonoalamide E or phosphonoalamide F.

Also provided are methods of reducing a bacterial population, the method comprising exposing the bacterial population to phosphonoalamide E or phosphonoalamide F.

Also provided are methods of preventing, inhibiting, ameliorating, and/or treating a bacterial infection in a plant, the method comprising exposing the plant to phosphonoalamide E or phosphonoalamide F, thereby reducing or eliminating the bacterial infection, including such methods wherein the plant is an onion plant.

Also provided are methods of preventing, inhibiting, ameliorating, and/or treating a bacterial infection in an animal, the method comprising exposing the animal to phosphonoalamide E or phosphonoalamide F, thereby reducing or eliminating the bacterial infection in the animal, including such methods wherein the animal is selected from the group consisting of: companion animal, livestock, research animal, and human.

Also provided are methods method of preventing, inhibiting, ameliorating, and/or treating a bacterial pathology in a honeybee population, comprising exposing a honeybee population to phosphonoalamide E or phosphonoalamide F, thereby reducing or eliminating the bacterial pathology in the honeybee population, including those methods, wherein the pathology is American foulbrood. In some examples, Serratia marcescens and/or Paenibacillus larvae contribute to the pathology.

Also provided are methods to prevent, inhibit, ameliorate, and/or treat bacterial pathology in plant, animal, or insect, comprising exposing the plant, animal, or insect to B. velezensis NRRL B-41850 and thereby prevent, inhibit, ameliorate, and/or treat bacterial pathology.

In some examples, the bacteria is Escherichia coli K12, Pseudomonas aeruginosa K, Salmonella enterica LT2, Serratia marcescens, or from the Bacillus family.

In some examples, the phosphonoalamide E or phosphonoalamide F are delivered via cultured Bacillus velezensis, including wherein the cell is Bacillus velezensis is genetically-engineered to produce phosphonoalamide E or phosphonoalamide F.

In some examples, exposure to phosphonoalamide E or phosphonoalamide F is via a composition herein, including those wherein Bacillus velezensis is genetically-engineered to produce phosphonoalamide E or phosphonoalamide F.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The compositions and methods of the appended claims are not limited in scope by the specific compositions methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A compound comprising a phosphonoalamide of Formula I and/or Formula II: 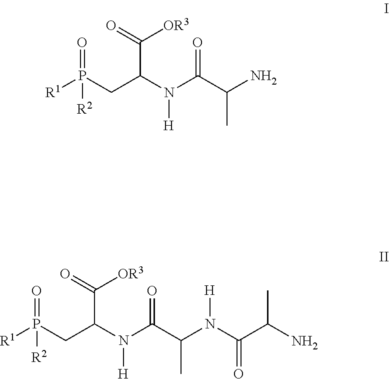
wherein
R¹ is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{a};
R² is hydrogen, halide, substituted or unsubstituted C₁-C₂₀ alkyl, substituted or unsubstituted C₃-C₂₀ cycloalkyl, substituted or unsubstituted C₁-C₂₀ alkoxy, substituted or unsubstituted C₃-C₂₀ aryl (e.g., substituted or unsubstituted phenyl), substituted or unsubstituted C₄-C₂₁ alkylaryl, NR^{x}R^{y}, or OR^{b};
R³ is hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl; and
R^{x} and R^{y} are independently selected from H, or substituted or unsubstituted C₁-C₅ alkyl, or substituted or unsubstituted C₁-C₅ acyl; or a derivative or salt thereof.

2. The compound of claim 1, wherein R¹ is OR^{a} and/or R² is OR^{b}.

3. The compound of claim 1, wherein the compound is of Formula I-A and/or Formula II-A: 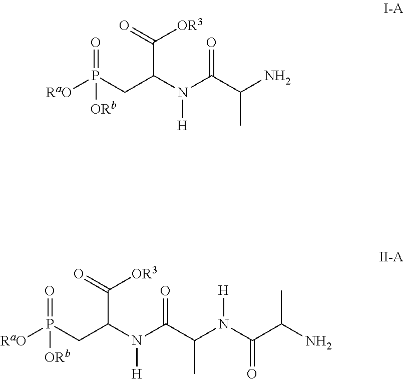
wherein
R^{a}, R^{b}, and R³ are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
or a derivative or salt thereof.

4. (canceled)

5. The compound of claim 1, wherein the compound is of Formula I-B and/or Formula II-B: 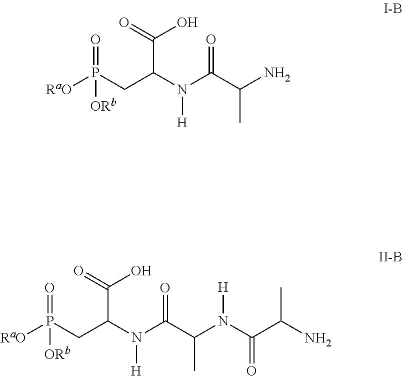
wherein
R^{a} and R^{b} are each independently hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
or a derivative or salt thereof.

6. (canceled)

7. The compound of claim 1, wherein the compound is of Formula I-C and/or Formula II-C: 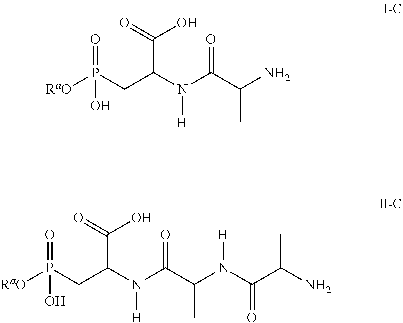
wherein
R^{a} is hydrogen, substituted or unsubstituted C₁-C₁₀ alkyl, substituted or unsubstituted C₃-C₁₀ aryl (e.g., substituted or unsubstituted phenyl), or substituted or unsubstituted C₄-C₁₁ alkylaryl;
or a derivative or salt thereof.

8-11. (canceled)

12. The compound of claim 1, wherein the compound is selected from the group consisting of: 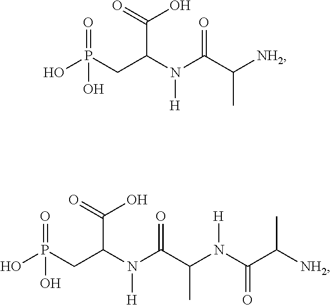 [SMILES: CC(N)C(=O)NC(CP(=O)(O)O)C(=O)O ; CC(N)C(=O)NC(C)C(=O)NC(CP(=O)(O)O)C(=O)O]
derivatives or salts thereof, and combinations thereof.

13. The compound of claim 1, wherein the compound is selected from the group consisting of: 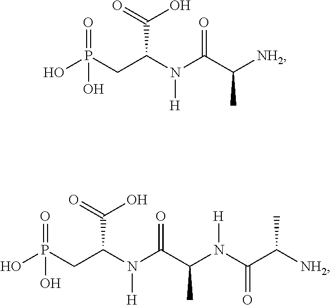 [SMILES: C[C@H](N)C(=O)N[C@H](CP(=O)(O)O)C(=O)O ; C[C@H](N)C(=O)N[C@@H](C)C(=O)N[C@H](CP(=O)(O)O)C(=O)O]
derivatives or salts thereof, and combinations thereof.

14. The compound of claim 1, wherein the compound is a salt.

15-20. (canceled)

21. The compound of claim 1, wherein the compound is an anti-metabolite.

22. The compound of claim 1, wherein the compound is a Bacillus isolate or a derivative or salt thereof.

23. The compound of claim 1, wherein the compound is an isolate of Bacillus velezensis NRRL B-41580, Bacillus subtilis NRRL B-4247, Bacillus swezeyi NRRL B-41282, Bacillus swezeyi NRRL B-41294, or a combination thereof; or a derivative or salt thereof.

24. (canceled)

25. (canceled)

26. A composition comprising the compound of claim 1.

27-33. (canceled)

34. The composition of claim 26, wherein the composition exhibits antimicrobial activity.

35-41. (canceled)

42. The composition of claim 26, wherein the composition is formulated for delivery to a plant or animal.

43-52. (canceled)

53. A cell comprising the compound of claim 1, wherein the cell comprises a Bacillus cell.

54-57. (canceled)

58. A method of use of the compound of claim 1, wherein the method comprises using the compound, composition, nucleic acid, vector, or cell as an antimicrobial, herbicide, pesticide, or combination thereof to control an undesirable population.

59-70. (canceled)

71. A method for treating, preventing, inhibiting, and/or ameliorating a microbial infection in a plant or a subject, comprising administering to the plant or subject an effective amount of the compound of claim 1.

72-83. (canceled)

84. The method of claim 71, wherein the compound is delivered via cultured Bacillus.

85-88. (canceled)

89. A method of isolating and/or purifying a phosphonate produced by a cell, wherein the phosphonate comprises the compound of claim 1.
